SEQUENCE LISTING (99 sequences)

SEQ ID NO: 1 - protein, 118 aa, Artificial Sequence
  mutant human beta-NGF
SSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVR

SEQ ID NO: 2 - protein, 120 aa, Artificial Sequence
  mutant human beta-NGF
SSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 3 - protein, 118 aa, Human
  wildtype human beta-NGF
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVR

SEQ ID NO: 4 - protein, 120 aa, Human
  wildtype human beta-NGF
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 5 - protein, 103 aa, Artificial Sequence
  NGF propeptide
EPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKR

SEQ ID NO: 6 - protein, 18 aa, Artificial Sequence
  NGF signal peptide
MSMLFYTLITAFLIGIQA

SEQ ID NO: 7 - protein, 232 aa, Human
  human wildtype IgG1 Fc IGHG1*05
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 232 aa, Human
  human wildtype IgG1 Fc IGHG1*03, natural variant
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 232 aa, Artificial Sequence
  modified IgG1 Fc M1
EPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 227 aa, Artificial Sequence
  modified IgG1 Fc M1-5
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 232 aa, Artificial Sequence
  modified IgG1 Fc M3
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 227 aa, Artificial Sequence
  modified IgG1 Fc M3-5
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 232 aa, Artificial Sequence
  modified IgG1 Fc M5
EPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 227 aa, Artificial Sequence
  modified IgG1 Fc M5-5
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 231 aa, Artificial Sequence
  modified IgG1 Fc M7
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 226 aa, Artificial Sequence
  modified IgG1 Fc M7-5
DKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 229 aa, Human
  human wildtype IgG4 Fc
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 229 aa, Artificial Sequence
  modified IgG4 Fc
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 228 aa, Artificial Sequence
  modified IgG4 Fc-FD
SKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 20 - protein, 229 aa, Artificial Sequence
  modified IgG2/4 Fc
VERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - DNA, 1464 bp, Artificial Sequence
  nucleic acid sequence of FD-G4Fc
atgagcatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaggctccggcggcggctccggtggcggcggctcaggaggaggaggctccggtggtggtggttcctccaagtatggccccccctgccccccctgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaa

SEQ ID NO: 22 - protein, 488 aa, Artificial Sequence
  amino acid sequence of FD-G4Fc
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRGSGGGSGGGGSGGGGSGGGGSSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 23 - DNA, 1434 bp, Artificial Sequence
  nucleic acid sequence of WM-G24Fc
atgagcatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaaagaccggcggtggctccggcggcggctccgtggagcggaagtgctgcgtggagtgccccccctgccccgctccccccgtggctggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 24 - protein, 478 aa, Artificial Sequence
  amino acid sequence of WM-G24Fc
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRKTGGGSGGGSVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - DNA, 1416 bp, Artificial Sequence
  nucleic acid sequence of NGF-1-15M7
atgagcatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagccgagcccaaatcttgtgacaaaactcacacatgcccaccgtgcccagcacctccagtcgcaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 26 - protein, 472 aa, Artificial Sequence
  amino acid sequence of NGF-1-15M7
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRAEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 1446 bp, Artificial Sequence
  nucleic acid sequence of NGF-L3Fc10M7-5
atgagcatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagccggcggtggcggctccggcggtggcggctccggcggtggcggctccgacaaaactcacacatgcccaccgtgcccagcacctcctgtcgccggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 28 - protein, 482 aa, Artificial Sequence
  amino acid sequence of NGF-L3Fc10M7-5
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRAGGGGSGGGGSGGGGSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1416 bp, Artificial Sequence
  nucleic acid sequence of 2-1-15M7
atgagcatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaagagtcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagccgagcccaaatcttgtgacaaaactcacacatgcccaccgtgcccagcacctccagtcgcaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 30 - protein, 472 aa, Artificial Sequence
  amino acid sequence of 2-1-15M7
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRAEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 1410 bp, Artificial Sequence
  nucleic acid sequence of NGF-4-12PAA
atgagcatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagccgagtccaaatatggtcccccatgcccaccctgcccagcacctgaggctgcggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 32 - protein, 470 aa, Artificial Sequence
  amino acid sequence of NGF-4-12PAA
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - DNA, 1443 bp, Artificial Sequence
  nucleic acid sequence of 2-118-L3Fc10-M1-5
atgagcatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaagagtcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaggcggtggcggctccggcggtggcggctccggcggtggcggctccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 34 - protein, 481 aa, Artificial Sequence
  amino acid sequence of 2-118-L3Fc10-M1-5
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 1443 bp, Artificial Sequence
  nucleic acid sequence of 2-118-L3Fc10-M3-5
atgagcatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaagagtcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaggcggtggcggctccggcggtggcggctccggcggtggcggctccgacaaaactcacacatgcccaccgtgcccagcacctgaagccgctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 36 - protein, 481 aa, Artificial Sequence
  amino acid sequence of 2-118-L3Fc10-M3-5
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 1443 bp, Artificial Sequence
  nucleic acid sequence of NGF-118-L3Fc10-M3-5
atgagcatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaggcggtggcggctccggcggtggcggctccggcggtggcggctccgacaaaactcacacatgcccaccgtgcccagcacctgaagccgctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 38 - protein, 481 aa, Artificial Sequence
  amino acid sequence of NGF-118-L3Fc10-M3-5
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 1449 bp, Artificial Sequence
  nucleic acid sequence of 2-L3Fc10-M3-5
atgagcatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaagagtcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagccggcggtggcggctccggcggtggcggctccggcggtggcggctccgacaaaactcacacatgcccaccgtgcccagcacctgaagccgctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 40 - protein, 483 aa, Artificial Sequence
  amino acid sequence of 2-L3Fc10-M3-5
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRAGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 1443 bp, Artificial Sequence
  nucleic acid sequence of 2-118-L3Fc10-M5-5
atgagcatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaagagtcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaggcggtggcggctccggcggtggcggctccggcggtggcggctccgacaaaactcacacatgcccaccgtgcccagcacctgaagctgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 42 - protein, 481 aa, Artificial Sequence
  amino acid sequence of 2-118-L3Fc10-M5-5
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRGGGGSGGGGSGGGGSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 1440 bp, Artificial Sequence
  nucleic acid sequence of 2-118-L3Fc10M7-5
atgagcatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaagagtcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaggcggtggcggctccggcggtggcggctccggcggtggcggctccgacaaaactcacacatgcccaccgtgcccagcacctcctgtcgccggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 44 - protein, 480 aa, Artificial Sequence
  amino acid sequence of 2-118-L3Fc10M7-5
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRGGGGSGGGGSGGGGSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - DNA, 1458 bp, Artificial Sequence
  nucleic acid sequence of 2-118-L3G4-BM
atgagcatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaagagtcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaggagggggaggcggaggttcagggggtggtggttccggcggcgggggatccgccgagtccaaatatggtcccccatgcccaccctgcccagcacctgaggctgcggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 46 - protein, 486 aa, Artificial Sequence
  amino acid sequence of 2-118-L3G4-BM
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 239 aa, Artificial Sequence
  mutant human preproNGF
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVR

SEQ ID NO: 48 - protein, 241 aa, Artificial Sequence
  mutant human preproNGF
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 49 - protein, 239 aa, Human
  wildtype human preproNGF
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVR

SEQ ID NO: 50 - protein, 241 aa, Human
  wildtype human preproNGF
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 51 - protein, 221 aa, Artificial Sequence
  mutant human proNGF
EPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVR

SEQ ID NO: 52 - protein, 223 aa, Artificial Sequence
  mutant human proNGF
EPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 53 - protein, 221 aa, Human
  wildtype human proNGF
EPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVR

SEQ ID NO: 54 - protein, 223 aa, Human
  wildtype human proNGF
EPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 55 - protein, 367 aa, Artificial Sequence
  amino acid sequence of mature FD-G4Fc
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRGSGGGSGGGGSGGGGSGGGGSSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 56 - protein, 357 aa, Artificial Sequence
  amino acid sequence of mature WM-G24Fc
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRKTGGGSGGGSVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 351 aa, Artificial Sequence
  amino acid sequence of mature NGF-1-15M7
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRAEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 361 aa, Artificial Sequence
  amino acid sequence of mature NGF-L3Fc10M7-5
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRAGGGGSGGGGSGGGGSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 351 aa, Artificial Sequence
  amino acid sequence of mature 2-1-15M7
SSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRAEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 349 aa, Artificial Sequence
  amino acid sequence of mature NGF-4-12PAA
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - protein, 360 aa, Artificial Sequence
  amino acid sequence of mature 2-118-L3Fc10-M1-5
SSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 360 aa, Artificial Sequence
  amino acid sequence of mature 2-118-L3Fc10-M3-5
SSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 360 aa, Artificial Sequence
  amino acid sequence of mature NGF-118-L3Fc10-M3-5
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 362 aa, Artificial Sequence
  amino acid sequence of mature 2-L3Fc10-M3-5
SSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRAGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 360 aa, Artificial Sequence
  amino acid sequence of mature 2-118-L3Fc10-M5-5
SSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRGGGGSGGGGSGGGGSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 359 aa, Artificial Sequence
  amino acid sequence of mature 2-118-L3Fc10M7-5
SSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRGGGGSGGGGSGGGGSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 365 aa, Artificial Sequence
  amino acid sequence of mature 2-118-L3G4-BM
SSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 68 - protein, 15 aa, Artificial Sequence
  linker
GGGGSGGGGSGGGGS

SEQ ID NO: 69 - protein, 18 aa, Artificial Sequence
  linker
GGGGGGSGGGGSGGGGSA

SEQ ID NO: 70 - protein, 5 aa, Artificial Sequence
  linker; (GGGGS)n, n is an integer of at least 1
GGGGS

SEQ ID NO: 71 - protein, 21 aa, Artificial Sequence
  linker
GSGGGSGGGGSGGGGSGGGGS

SEQ ID NO: 72 - protein, 10 aa, Artificial Sequence
  linker
KTGGGSGGGS

SEQ ID NO: 73 - protein, 1 aa, Artificial Sequence
  linker; (G)n, n is an integer of at least 1
G

SEQ ID NO: 74 - protein, 2 aa, Artificial Sequence
  linker; (GS)n, n is an integer of at least 1
GS

SEQ ID NO: 75 - protein, 3 aa, Artificial Sequence
  linker; (GGS)n, n is an integer of at least 1
GGS

SEQ ID NO: 76 - protein, 4 aa, Artificial Sequence
  linker; (GGGS)n, n is an integer of at least 1
GGGS

SEQ ID NO: 77 - protein, 7 aa, Artificial Sequence
  linker; (GGS)n(GGGS)n, n is an integer of at least 1
GGSGGGS

SEQ ID NO: 78 - protein, 5 aa, Artificial Sequence
  linker; (GSGGS)n, n is an integer of at least 1
GSGGS

SEQ ID NO: 79 - protein, 5 aa, Artificial Sequence
  linker; (GGSGS)n, n is an integer of at least 1
GGSGS

SEQ ID NO: 80 - protein, 16 aa, Artificial Sequence
  linker
GSGGGSGGGGSGGGGS

SEQ ID NO: 81 - protein, 14 aa, Artificial Sequence
  linker
GGGSGGGGSGGGGS

SEQ ID NO: 82 - protein, 10 aa, Artificial Sequence
  linker
GGGSGGSGGS

SEQ ID NO: 83 - protein, 15 aa, Artificial Sequence
  linker
GGSGGSGGSGGSGGG

SEQ ID NO: 84 - protein, 16 aa, Artificial Sequence
  linker
GGSGGSGGGGSGGGGS

SEQ ID NO: 85 - protein, 18 aa, Artificial Sequence
  linker
GGSGGSGGSGGSGGSGGS

SEQ ID NO: 86 - protein, 2 aa, Artificial Sequence
  linker
GG

SEQ ID NO: 87 - protein, 4 aa, Artificial Sequence
  linker
GGSG

SEQ ID NO: 88 - protein, 5 aa, Artificial Sequence
  linker
GGSGG

SEQ ID NO: 89 - protein, 5 aa, Artificial Sequence
  linker
GSGSG

SEQ ID NO: 90 - protein, 5 aa, Artificial Sequence
  linker
GSGGG

SEQ ID NO: 91 - protein, 5 aa, Artificial Sequence
  linker
GGGSG

SEQ ID NO: 92 - protein, 5 aa, Artificial Sequence
  linker
GSSSG

SEQ ID NO: 93 - protein, 6 aa, Artificial Sequence
  linker
GGSGGS

SEQ ID NO: 94 - protein, 6 aa, Artificial Sequence
  linker
SGGGGS

SEQ ID NO: 95 - protein, 5 aa, Artificial Sequence
  linker
GGGGS

SEQ ID NO: 96 - protein, 2 aa, Artificial Sequence
  linker; (GA)n, n is an integer of at least 1
GA

SEQ ID NO: 97 - protein, 12 aa, Artificial Sequence
  linker
GRAGGGGAGGGG

SEQ ID NO: 98 - protein, 6 aa, Artificial Sequence
  linker
GRAGGG

SEQ ID NO: 99 - protein, 6 aa, Artificial Sequence
  linker
ASTKGP

(19) United States
(12) Patent Application Publication
(10) Pub. No.: US 2024/0400629 A1
(43) Pub. Date: Dec. 5, 2024

(54) LONG-ACTING NERVE GROWTH FACTOR POLYPEPTIDES AND USES THEREOF

(71) Applicant: STAIDSON (BEIJING) BIOPHARMACEUTICALS CO., LTD., Beijing (CN)

(72) Inventors: Ping LI, Beijing (CN); Lei ZHANG, Beijing (CN)

(73) Assignee: STAIDSON (BEIJING) BIOPHARMACEUTICALS CO., LTD., Beijing (CN)

(21) Appl. No.: 18/037,503

(22) PCT Filed: Nov. 19, 2021
(86) PCT No.: PCT/CN2021/131614

§ 371 Date: May 17, 2023

(30) Foreign Application Priority Data
Nov. 19, 2020 (WO) PCT/CN2020/129925

(51) Int. Cl. C07K 14/48; A61K 38/00; A61P 15/08; A61P 25/28; A61P 27/02

(52) U.S. Cl. CPC C07K 14/48; A61P 15/08; A61P 25/28; A61P 27/02; A61K 38/00; C07K 2319/30

(57) ABSTRACT

Provided are long-acting nerve growth factor (NGF) polypeptides comprising from N-terminus to C-terminus an NGF moiety and an Fe moiety, methods of making, and uses thereof.

### CROSS REFERENCE TO RELATED APPLICATIONS

This is a U.S. National Phase application based upon International Patent Applications No. PCT/CN2021/131614, filed on Nov. 19, 2021, which claims priority benefit of International Patent Application No. PCT/CN2020/129925, filed on Nov. 19, 2020, the contents of which are incorporated herein by reference in their entireties.

### INCORPORATION OF SEQUENCE LISTING

The sequence listing that is contained in the file named “UNITP0104US-Sequence_Listing”, which is 170 KB (as measured in Microsoft Windows®) and created on Sep. 27, 2023, is filed herewith by electronic submission, and is incorporated by reference herein.

### FIELD OF THE INVENTION

The present invention relates to long-acting nerve growth factor (NGF) polypeptides comprising from N-terminus to C-terminus an NGF moiety and an Fc moiety, methods of making, and uses thereof.

### BACKGROUND OF THE INVENTION

Neurotrophins are a highly homologous family of growth factors that are crucial for survival and maintenance of neurons during developmental and adult stages of the vertebrate nervous system. Limited neurotrophin production can lead to degeneration or death of neurons either in peripheral nervous system (PNS) or in central nervous system (CNS).

Nerve Growth Factor (NGF) is the first discovered member of the neurotrophin family, found in mouse sarcoma cells by Italian scientist Levi-Montlcini in 1953. NGF is a neuronal growth regulator having a dual biological function of neuron nutrition and promoting neurite growth, which plays an important regulatory role in the development, differentiation, growth, regeneration, and expression of functional properties of central and peripheral neurons. NGF includes three subunits of α, β, and γ. The β subunit is an active region, which is formed by combining two single chains through a non-covalent bond.

NGF has been studied for decades. However, very few NGF products are on the market, most of them are mainly used for the treatment of ophthalmic diseases, including corneal ulcer, optic contusion, and optive injuries. The underlying reasons lie in the limitations and problems in actual application.

Like other proteins, the biological activity of NGF depends on the secondary and tertiary structures thereof, and thus it is particularly important to maintain its biological activity during preparation, purification, storage and administration.

Further, NGF may cause pain, which cannot be tolerated by some patients during application, thereby partially limiting its use. Pain may be divided into two types: sensory pain and neuropathic pain according to its neurophysiological mechanism. The former is directly caused by noxious stimulation, relates to tissue damage or inflammatory reaction, and is also known as inflammatory pain. The latter is a chronic pain directly caused by the damage or disease of somatosensory nervous system. NGF is involved in the pathophysiological process of pain through affecting the release of inflammatory mediators, the opening of ion channels, and promoting the growth of nerve fibers to cause pain; and is involved in the development of pain through regulating ion channels and molecular signals. Some scholars speculate that NGF may also cause pain through promoting the expression of pain-inducing substances, and may change the budding and regeneration of neurons after injury of organism. Research indicates that the maximum dose that does not cause hyperalgesia in humans is about 0.03 μg/kg (Petty et al., Ann Neurol. 1994; 36 (2): 244-246). However, such low dose limits the application of NGF and also limits the expansion of its indications, such as use for the central nervous system.

As a protein drug, the active portion of NGF in promoting nerve growth resides mainly in β-NGF. β-NGF has a sedimentation coefficient of 2.5S, a molecular weight of 13.5 KDa, and is easily filtered by glomerulus during metabolism, resulting in a short half-life in vivo. Studies have shown that mice intramuscularly administered with β-NGF drug has T1/2 (β)=2.2 h, Tmax=0.5 h, and the frequency of injection was once a day. Due to the adverse pain reactions at the injection site or in the lower limb on the injection side during NGF injection, reduction in the total number and frequency of administration would be preferred.

The disclosures of all publications, patents, patent applications and published patent applications referred to herein are hereby incorporated herein by reference in their entirety.

### BRIEF SUMMARY OF THE INVENTION

One aspect of the present application provides a long-acting nerve growth factor (NGF) polypeptide comprising from N-terminus to C-terminus an NGF moiety and an Fc moiety, wherein the NGF moiety comprises the amino acid sequence of any one of SEQ ID NOs: 1-4 (such as any one of SEQ ID NOs: 1-3), and wherein the Fc moiety is derived from an IgG1 Fc or an IgG4 Fc.

In some embodiments according to any one of the long-acting NGF polypeptides described above, the NGF moiety is fused to the Fc moiety via a peptide linker. In some embodiments, the peptide linker comprises the amino acid sequence of any one of SEQ ID NOs: 68-99, such as any one of SEQ ID NOs: 68-72, or SEQ ID NO: 68 or 69. In some embodiments, the peptide linker comprises the amino acid sequence of (GGGGS)n (SEQ ID NO: 70), wherein n is any of 1, 2, 3, 4, 5, or 6.

In some embodiments according to any one of the long-acting NGF polypeptides described above, the Fc moiety is derived from an IgG1 Fc comprising the amino acid sequence of SEQ ID NO: 7 or 8. In some embodiments, the Fc moiety comprises a mutation at a position selected from one or more of E233, L234, L235, G236, G237, N297, A327, A330, and P331 relative to SEQ ID NO: 8. In some embodiments, the Fc moiety comprises a mutation selected from one or more of E233P, L234V, L234A, L235A, L235E, G236del, G237A, N297A, A327G, A330S, and P331S relative to SEQ ID NO: 8. In some embodiments, the Fc moiety further lacks the first 5 amino acids of SEQ ID NO: 7 or 8. In some embodiments, the Fc moiety comprises L234A, L235A, and P331S mutations relative to SEQ ID NO: 8. In some embodiments, the Fc moiety comprises the amino acid sequence of SEQ ID NO: 11 or 12. In some embodiments, the long-acting NGF polypeptide comprises the amino acid sequence of any one of SEQ ID NOs: 62-64. In some embodiments, the Fc moiety comprises E233P, L234V, L235A, G236del, A327G, A330S, and P331S mutations relative to SEQ ID NO: 8. In some embodiments, the Fc moiety comprises the amino acid sequence of SEQ ID NO: 15 or 16. In some embodiments, the long-acting NGF polypeptide comprises the amino acid sequence of SEQ ID NO: 66. In some embodiments, the Fc moiety comprises L234A, L235E, G237A, A330S, and P331S mutations relative to SEQ ID NO: 8. In some embodiments, the Fc moiety comprises the amino acid sequence of SEQ ID NO: 13 or 14. In some embodiments, the long-acting NGF polypeptide comprises the amino acid sequence of SEQ ID NO: 65. In some embodiments, the Fc moiety comprises an N297A mutation relative to SEQ ID NO: 8. In some embodiments, the Fc moiety comprises the amino acid sequence of SEQ ID NO: 9 or 10. In some embodiments, the long-acting NGF polypeptide comprises the amino acid sequence of SEQ ID NO: 61.

In some embodiments according to any one of the long-acting NGF polypeptides described above, the Fc moiety is derived from an IgG4 Fc comprising the amino acid sequence of SEQ ID NO: 17. In some embodiments, the Fc moiety comprises a mutation at a position selected from one or more of S228, F234, and L235 relative to SEQ ID NO: 17. In some embodiments, the Fc moiety comprises a mutation selected from one or more of S228P, F234A, and L235A relative to SEQ ID NO: 17. In some embodiments, the Fc moiety comprises the amino acid sequence of SEQ ID NO: 18. In some embodiments, the long-acting NGF polypeptide comprises the amino acid sequence of SEQ ID NO: 67.

In some embodiments according to any one of the long-acting NGF polypeptides described above, the long-acting NGF polypeptide has a half-life of at least about 10 hours (e.g., at least about any of 11, 12, 13, 14, 15, 16, 17, 18, 19, 20, 21, 22, 23, 24, 25, 30, 35, 40, 45, 50, 55, 60, 70, 80, 90, or 100 hours) when administered to an individual (e.g., human) intravenously, intramuscularly, or subcutaneously.

In some embodiments according to any one of the long-acting NGF polypeptides described above, the long-acting NGF polypeptide causes less pain (e.g., at least about 10%, 20%, 30%, 40%, 50%, 60%, 70%, 80%, 90%, or 100% less pain) compared to an NGF polypeptide comprising an NGF moiety with the amino acid sequence of SEQ ID NO: 4.

Also provided are isolated nucleic acids encoding any of the long-acting NGF polypeptides described herein, vectors comprising such nucleic acids, host cell (e.g., CHO cells, HEK 293 cells, Hela cells, or COS cells) comprising such nucleic acids or vectors, compositions (e.g., pharmaceutical compositions), kits, and articles of manufacture comprising any of the long-acting NGF polypeptides described herein. Methods of treating an NGF-related disease (e.g., neurological disease or non-neurological disease) in an individual (e.g., human) using any of the long-acting NGF polypeptides described herein or pharmaceutical compositions thereof are also provided.

### BRIEF DESCRIPTION OF THE DRAWINGS

FIGS. 1A-1F depict sequence alignments for human wildtype IgG1 Fc IGHG1*05 and IgG1 Fc natural variant IGHG1*03 (FIG. 1A), modified IgG1 Fc M1-5 and IGHG1*03 (FIG. 1B), modified IgG1 Fc M3-5 and IGHG1*03 (FIG. 1C), modified IgG1 Fc M5-5 and IGHG1*03 (FIG. 1D), modified IgG1 Fc M7 and IGHG1*03 (FIG. 1E), and modified IgG4 Fc and human wildtype IgG4 Fc (FIG. 1F).

FIG. 2 depicts the structure of preproNGF containing a signal peptide (SP), a propeptide, and a mature NGF. Furin cleavage at the major cleavage site is responsible for processing of proNGF to mature NGF.

FIGS. 3A-3M depict Size Exclusion Chromatography (SEC) measurement of aggregate %, fragment %, and monomer % of various mature NGF-Fc fusion proteins under 40° C. accelerated stability test at different time points.

FIGS. 4A-4M depict Capillary Electrophoresis-Sodium Dodecyl Sulfate (CE-SDS) measurement of various mature NGF-Fc fusion proteins under 40° C. accelerated stability test at different time points.

FIGS. 5A-5B depict TF-1 cell proliferation activity treated by various NGF-Fc fusion proteins. SuTaiSheng® mouse NGF, mutant NGF 118aa (mNGF118), and recombinant human NGF (rhNGF) served as controls.

FIGS. 6A-6D depict the bioactivity of various NGF-Fc fusion proteins on superior cervical ganglion (SCG) in vivo growth in rats. SuTaiSheng® mouse NGF and mNGF118 served as controls. PBS served as negative control.

FIG. 7A depicts pharmacokinetic (PK) profiles of 2-118-L3Fc10-M3-5, 2-118-L3G4-BM, and mNGF118 control (mutant β-NGF 118aa without Fc fusion) in blood plasma. FIG. 7B depicts their half-life in vivo when intramuscularly injected at 235 μg/kg.

FIG. 8 depicts wound closure rate of diabetic mice treated with NGF (SuTaiSheng® mouse NGF or mNGF118) or NGF-Fc fusion protein (2-118-L3Fc10-M3-5 or 2-118-L3G4-BM). PBS treatment served as negative control.

FIGS. 9A-9B depict the proliferation rate and the secreted estrogen concentration of human ovarian granulosa-like tumor cell line (KGN) treated with NGF (SuTaiSheng® mouse NGF or mNGF118) or NGF-Fc fusion protein (2-118-L3Fc10-M3-5 or 2-118-L3G4-BM).

FIG. 9C depicts the number of follicles in rat model of premature ovarian failure treated with NGF (SuTaiSheng® mouse NGF or mNGF118) or NGF-Fc fusion protein (2-118-L3Fc10-M3-5 or 2-118-L3G4-BM).

FIGS. 10A and 10B depict corneal fluorescein sodium staining score (FIG. 10A) and average corneal nerve length (FIG. 10B) of rat models of neurotrophic keratitis treated with NGF (SuTaiSheng® mouse NGF or mNGF118), NGF-Fc fusion protein (2-118-L3Fc10-M3-5 or 2-118-L3G4-BM), or 0.9% sodium chloride solution as negative control.

### DETAILED DESCRIPTION OF THE INVENTION

The present invention provides long-acting NGF polypeptides comprising from N-terminus to C-terminus an NGF moiety and an Fc moiety. The terms “long-acting NGF polypeptide,” “long-acting NGF-Fc fusion protein,” and “long-acting NGF construct” are used herein interchangeably.

NGF plays an important regulatory role in the development, differentiation, growth, regeneration, and expression of functional properties of central and peripheral neurons. It has been used for the treatment of nervous system dysplasia, including amblyopia, neuroma, various nerve injuries and nervous system diseases. However, its adverse effects such as causing pain, short in vivo half-life, low dose limit in order to avoid hyperalgesia, and frequent administration regime limits NGF's widespread application. Fusing a protein drug to a moiety having a longer half-life and/or larger molecular weight is a strategy of enabling long-acting activity of certain protein drugs. However, it is still a clinically difficult problem to increase or maintain the biological activity of the protein drug while prolonging its half-life.

The long-acting NGF polypeptides described herein have one or more of the following superior effects: 1) they are highly biologically active (e.g., promoting superior cervical ganglion growth), both in vitro and in vivo, even better than available NGF-Fc fusion proteins or NGF drugs; 2) they have very long in vivo half-life, not only much longer than NGF proteins without fusion moiety, but also significantly longer than available NGF-Fc fusion proteins, thereby reducing the administration frequency and total administration number, and providing convenience and reduced cost to the patients; 3) they can alleviate side effects such as pain, or are even painlessness, thereby increasing the dosage tolerable by patients, and providing the possibility to expand the indications and apply to the central nervous system; 4) they have reduced or minimal antibody dependent cell-mediated cytotoxicity (ADCC) and/or complement dependent cytotoxicity (CDC), thereby avoiding undesired immune responses during treatment; 5) they have excellent thermal stability (e.g., high melting temperature (Tm) and/or high onset aggregation temperature (Tagg)); 6) they have superior stability under accelerated stress (e.g., heating), such as less or no fragmentation, aggregate formation, and/or aggregate increment, thereby maintaining drug property; and 7) they are very effective in treating NGF-related disease in vivo, for example, neurological diseases such as diabetic neuropathy, Alzheimer's disease, and neurotrophic keratitis, non-neurological diseases such as premature ovarian failure and spermatogenesis disorder (e.g., oligozoospermia, asthenospermia, oligoasthenospermia), with comparable or even better therapeutic efficacy compared to non-Fc fused NGF moiety.

Accordingly, one aspect of the present application provides long-acting NGF polypeptides comprising from N-terminus to C-terminus an NGF moiety and an Fc moiety, wherein the NGF moiety comprises the amino acid sequence of any one of SEQ ID NOs: 1-4, and wherein the Fc moiety is derived from an IgG1 Fc or an IgG4 Fc.

Also provided are isolated nucleic acids encoding such long-acting NGF polypeptides, vectors comprising such nucleic acids, host cells comprising such nucleic acids or vectors, methods of producing such long-acting NGF polypeptides, pharmaceutical compositions and articles of manufacture comprising such long-acting NGF polypeptides, and methods of treating diseases (e.g., neurological diseases associated with neuron degeneration or damage, such as diabetic neuropathy, Alzheimer's disease, and neurotrophic keratitis, non-neurological diseases such as premature ovarian failure and spermatogenesis disorder) with such long-acting NGF polypeptides or pharmaceutical compositions thereof.

### I. Definitions

The practice of the present invention will employ, unless indicated specifically to the contrary, conventional methods of virology, immunology, microbiology, molecular biology and recombinant DNA techniques within the skills of the art, many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3rd ed., John Wiley & Sons, 1995; Sambrook and Russell, Molecular Cloning: A Laboratory Manual (3rd Edition, 2001); Maniatis et al., Molecular Cloning: A Laboratory Manual (1982); DNA Cloning: A Practical Approach, vol. I&II (D. Glover, ed.); Oligonucleotide Synthesis (N. Gait, ed., 1984); Nucleic Acid Hybridization (β. Hames & S. Higgins, eds., 1985); Transcription and Translation (β. Hames & S. Higgins, eds., 1984); Animal Cell Culture (R. Freshney, ed., 1986); Perbal, A Practical Guide to Molecular Cloning (1984) and other like references.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the invention contemplate any one or more of these aspects of treatment. For example, an individual is successfully “treated” if one or more symptoms associated with the disease are mitigated or eliminated, including, but are not limited to, decreasing symptoms resulting from the disease, increasing the quality of life of those suffering from the disease, decreasing the dose of other medications required to treat the disease, and/or prolonging survival of individuals.

The term “prevent,” and similar words such as “prevented,” “preventing” etc., indicate an approach for preventing, inhibiting, or reducing the likelihood of the recurrence of, a disease or condition. It also refers to delaying the recurrence of a disease or condition or delaying the recurrence of the symptoms of a disease or condition. As used herein, “prevention” and similar words also includes reducing the intensity, effect, symptoms and/or burden of a disease or condition prior to recurrence of the disease or condition.

As used herein, “delaying” the development of a disease means to defer, hinder, slow, retard, stabilize, and/or postpone development of the disease. This delay can be of varying lengths of time, depending on the history of the disease and/or individual being treated. A method that “delays” development of a disease is a method that reduces probability of disease development in a given time frame and/or reduces the extent of the disease in a given time frame, when compared to not using the method. Such comparisons are typically based on clinical studies, using a statistically significant number of individuals.

The term “effective amount” used herein refers to an amount of an agent or a combination of agents, sufficient to treat a specified disorder, condition or disease such as ameliorate, palliate, lessen, and/or delay one or more of its symptoms. In some embodiments, an effective amount is an amount sufficient to delay disease development. In some embodiments, an effective amount is an amount sufficient to prevent or delay disease recurrence. An effective amount can be administered in one or more administrations. In some embodiments, the effective amount of the drug or composition may: (i) support neuronal survival; (ii) promote neurite outgrowth; (iii) enhance neurochemical differentiation; (iv) promote the proliferation of pancreatic β cells; (v) induces innate and/or acquired immunity; (vi) prevent or delay occurrence and/or recurrence of a disease; and/or (vii) relieve to some extent one or more of the symptoms associated with the disease.

As used herein, an “individual” or a “subject” refers to a mammal, including, but not limited to, human, bovine, horse, feline, canine, rodent, or primate. In some embodiments, the individual is a human.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, C_{H}) of the heavy chain and the CHL (or C_{L}) domain of the light chain. Depending on the amino acid sequence of the constant domain of immunoglobulin heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1 and IgA2.

The term “Fc region,” “fragment crystallizable region,” “Fc domain,” or “Fc moiety” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the protein, or by recombinantly engineering the nucleic acid encoding the protein. Suitable native-sequence Fc regions for use in the constructs described herein include human IgG1, IgG2 (IgG2A, IgG2B), IgG3, and IgG4.

The term IgG “isotype” or “subclass” as used herein is meant any of the subclasses of immunoglobulins defined by the chemical and antigenic characteristics of their constant regions. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000).

“Fc receptor” or “FcR” describes a receptor that binds the Fc region of an Fc-containing construct (e.g., antibody). The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See M. Daëron, Annu. Rev. Immunol. 15:203-234 (1997). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al., J. Immunol. 117:587 (1976) and Kim et al., J. Immunol. 24:249 (1994). Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9 (2): 6591-6604 (2001).

“Antibody effector functions” refer to those biological activities attributable to the Fc region (a native sequence Fc region or amino acid sequence variant Fc region) of an Fc-containing construct (e.g., antibody), and vary with Fc isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g., B cell receptors); and B cell activation. “Reduced or minimized” antibody effector function means that which is reduced by at least 50% (alternatively 60%, 65%, 70%, 75%, 80%, 85%, 90%, 95%, 96%, 97%, 98%, or 99%) from the wild type or unmodified Fc-containing construct (e.g., antibody). The determination of antibody effector function is readily determinable and measurable by one of ordinary skill in the art. In a preferred embodiment, the antibody effector functions of complement binding, complement dependent cytotoxicity and antibody dependent cytotoxicity are affected. In some embodiments, effector function is eliminated through a mutation in the constant region that eliminated glycosylation, e.g., “effectorless mutation.” In some embodiments, the effectorless mutation is an N297A or DANA mutation (D265A+N297A) in the C_{H}2 region. Shields et al., J. Biol. Chem. 276 (9): 6591-6604 (2001). Alternatively, additional mutations resulting in reduced or eliminated effector function include: K322A and L234A/L235A (LALA). Alternatively, effector function can be reduced or eliminated through production techniques, such as expression in host cells that do not glycosylate (e.g., E. coli.) or in which result in an altered glycosylation pattern that is ineffective or less effective at promoting effector function (e.g., Shinkawa et al., J. Biol. Chem. 278 (5): 3466-3473 (2003).

“Antibody-dependent cell-mediated cytotoxicity” or ADCC refers to a form of cytotoxicity in which secreted Ig (or Ligand-Fc construct) bound onto Fc receptors (FcRs) present on certain cytotoxic cells (e.g., natural killer (NK) cells, neutrophils and macrophages) enable these cytotoxic effector cells to bind specifically to an antigen-bearing (or ligand receptor-bearing) target cell and subsequently kill the target cell with cytotoxins. The antibodies (or Fc-containing constructs) “arm” the cytotoxic cells and are required for killing of the target cell by this mechanism. The primary cells for mediating ADCC, NK cells, express FcγRIII only, whereas monocytes express FcγRI, FcγRII, and FcγRIII. Fc expression on hematopoietic cells is summarized in Table 2 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998).

“Complement dependent cytotoxicity” or “CDC” refers to the lysis of a target cell in the presence of complement. Activation of the classical complement pathway is initiated by the binding of the first component of the complement system (Clq) to Fc-containing constructs (of the appropriate subclass) which are bound to their cognate receptor through the ligand fused to Fc. To assess complement activation, a CDC assay, e.g., as described in Gazzano-Santoro et al., J. Immunol. Methods 202:163 (1996), may be performed. Antibody variants with altered Fc region amino acid sequences and increased or decreased Clq binding capability are described in U.S. Pat. No. 6,194,551B1 and WO99/51642. The contents of those patent publications are specifically incorporated herein by reference. See, also, Idusogie et al. J. Immunol. 164:4178-4184 (2000).

As used herein, the term “specifically binds,” “specifically recognizes,” or is “specific for” refers to measurable and reproducible interactions such as binding between a ligand and a receptor, which is determinative of the presence of the ligand in the presence of a heterogeneous population of molecules including biological molecules. For example, a ligand that specifically binds a receptor is a ligand that binds this receptor with greater affinity, avidity, more readily, and/or with greater duration than it binds other receptors. In some embodiments, the extent of binding of a ligand to an unrelated receptor is less than about 10% of the binding of the ligand to the target receptor as measured, e.g., by a radioimmunoassay (RIA). In some embodiments, a ligand that specifically binds a target receptor has an equilibrium dissociation constant (K_{d}) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, a ligand specifically binds a receptor that is conserved among the receptors from different species. In some embodiments, specific binding can include, but does not require exclusive binding. Binding specificity of a ligand can be determined experimentally by methods known in the art. Such methods comprise, but are not limited to Western blots, ELISA-, RIA-, ECL-, IRMA-, EIA-, BIACORE™-tests and peptide scans.

“Binding affinity” generally refers to the strength of the sum total of non-covalent interactions between a single binding site of a molecule (e.g., ligand) and its binding partner (e.g., receptor). Unless indicated otherwise, as used herein, “binding affinity” refers to intrinsic binding affinity that reflects a 1:1 interaction between members of a binding pair. Binding affinity can be indicated by K_{d}, K_{off}, Kₒₙ, or Kₐ. The term “K_{off}”, as used herein, is intended to refer to the off rate constant for dissociation of a ligand from the ligand/receptor complex, as determined from a kinetic selection set up, expressed in units of s⁻¹. The term “Kₒₙ”, as used herein, is intended to refer to the on rate constant for association of a ligand to the receptor to form the ligand/receptor complex, expressed in units of M⁻¹s⁻¹. The term equilibrium dissociation constant “K_{d}”, as used herein, refers to the dissociation constant of a particular ligand-receptor interaction, and describes the concentration of ligand required to occupy one half of all of the receptors present in a solution of receptors at equilibrium, and is equal to K_{off}/Kₒₙ, expressed in units of M. The measurement of K_{d} presupposes that all binding agents are in solution. In the case where the receptor is on a cell membrane, the corresponding equilibrium rate constant is expressed as EC50, which gives a good approximation of K_{d}. The affinity constant, Kₐ, is the inverse of the dissociation constant, K_{d}, expressed in units of M⁻¹. The dissociation constant (K_{d}) is used as an indicator showing affinity of ligand to receptor. The K_{d} value that can be derived using these methods is expressed in units of M (mol/L).

Half maximal inhibitory concentration (IC₅₀) is a measure of the effectiveness of a substance (e.g., ligand) in inhibiting a specific biological or biochemical function. It indicates how much of a particular drug or other substance (inhibitor, e.g., ligand) is needed to inhibit a given biological process by half. The values are typically expressed as molar concentration. IC₅₀ is comparable to an “EC₅₀” for agonist drug or other substance (e.g., ligand). EC₅₀ also represents the plasma concentration required for obtaining 50% of a maximum effect in vivo. As used herein, an “IC₅₀” is used to indicate the effective concentration of a ligand needed to neutralize 50% of the receptor bioactivity in vitro. IC₅₀ or EC₅₀ can be measured by bioassays such as inhibition of ligand binding by FACS analysis (competition binding assay), cell based cytokine release assay, or amplified luminescent proximity homogeneous assay (AlphaLISA).

“Covalent bond” as used herein refers to a stable bond between two atoms sharing one or more electrons. Examples of covalent bonds include, but are not limited to, peptide bonds and disulfide bonds. As used herein, “peptide bond” refers to a covalent bond formed between a carboxyl group of an amino acid and an amine group of an adjacent amino acid. A “disulfide bond” as used herein refers to a covalent bond formed between two sulfur atoms, such as a combination of two Fc fragments by one or more disulfide bonds. One or more disulfide bonds may be formed between the two fragments by linking the thiol groups in the two fragments. In some embodiments, one or more disulfide bonds can be formed between one or more cysteines of two Fc fragments. Disulfide bonds can be formed by oxidation of two thiol groups. In some embodiments, the covalent linkage is directly linked by a covalent bond. In some embodiments, the covalent linkage is directly linked by a peptide bond or a disulfide bond.

“Percent (%) amino acid sequence identity” and “homology” with respect to a peptide or polypeptide sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

As used herein, the “C terminus” of a polypeptide refers to the last amino acid residue of the polypeptide which donates its amine group to form a peptide bond with the carboxyl group of its adjacent amino acid residue. “N terminus” of a polypeptide as used herein refers to the first amino acid of the polypeptide which donates its carboxyl group to form a peptide bond with the amine group of its adjacent amino acid residue.

An “isolated” polypeptide is one that has been identified, separated and/or recovered from a component of its production environment (e.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the polypeptide, and may include enzymes, hormones, and other proteinaceous or non-proteinaceous solutes. In some embodiments, the polypeptide will be purified: (1) to greater than 95% by weight of polypeptides as determined by, for example, the Lowry method, and in some embodiments, to greater than 99% by weight; (2) to a degree sufficient to obtain at least 15 residues of N-terminal or internal amino acid sequence by use of a spinning cup sequenator; or (3) to homogeneity by SDS-PAGE under non-reducing or reducing conditions using Coomassie Blue or, preferably, silver stain. Isolated polypeptide includes the polypeptide in situ within recombinant cells since at least one component of the polypeptide's natural environment will not be present. Ordinarily, however, an isolated polypeptide will be prepared by at least one purification step.

An “isolated” nucleic acid molecule encoding a construct (such as the NGF polypeptides described herein) is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides described herein is in a form other than in the form or setting in which it is found in nature. Isolated nucleic acid molecules therefore are distinguished from nucleic acid encoding the polypeptides described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that has the same function or biological activity as screened or selected for in the originally transformed cell are included herein.

The term “pharmaceutical formulation” of “pharmaceutical composition” refers to a preparation that is in such form as to permit the biological activity of the active ingredient to be effective, and that contains no additional components that are unacceptably toxic to a subject to which the formulation would be administered. Such formulations are sterile. A “sterile” formulation is aseptic or free from all living microorganisms and their spores.

It is understood that embodiments of the invention described herein include “consisting of” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat disease of type X means the method is used to treat disease of types other than X.

The term “about X-Y” used herein has the same meaning as “about X to about Y.”

As used herein and in the appended claims, the singular forms “a,” “or,” and “the” include plural referents unless the context clearly dictates otherwise.

### II. Long-Acting NGF Polypeptides

One aspect of the present application provides long-acting NGF polypeptides comprising from N-terminus to C-terminus an NGF moiety and an Fc moiety. In some embodiments, there is provided a long-acting NGF polypeptide comprising from N-terminus to C-terminus an NGF moiety and an Fc moiety, wherein the NGF moiety comprises (or consists essentially of, or consists of) the amino acid sequence of any one of SEQ ID NOs: 1-4 (e.g., any of SEQ ID NOs: 1-3), and wherein the Fc moiety is derived from an IgG1 Fc or an IgG4 Fc. In some embodiments, the NGF moiety comprises (or consists essentially of, or consists of) the amino acid sequence of SEQ ID NO: 1. In some embodiments, the long-acting NGF polypeptide has a half-life of at least about 10 hours (e.g., at least about any of 11, 12, 13, 14, 15, 16, 17, 18, 19, 20, 21, 22, 23, 24, 25, 30, 35, 40, 45, 50, 55, 60, 70, 80, 90, or 100 hours) when administered to an individual (e.g., human) intravenously, intramuscularly, or subcutaneously. In some embodiments, the long-acting NGF polypeptide causes less pain (e.g., at least about 10%, 20%, 30%, 40%, 50%, 60%, 70%, 80%, 90%, or 100% less pain) compared to an NGF moiety comprising the amino acid sequence of SEQ ID NO: 4.

In some embodiments, the NGF moiety is fused to the Fc moiety via a peptide linker. Thus in some embodiments, there is provided a long-acting NGF polypeptide comprising from N-terminus to C-terminus an NGF moiety, a peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), and wherein the Fc moiety is derived from an IgG1 Fc or an IgG4 Fc. In some embodiments, the peptide linker comprises (or consists essentially of, or consists of) the amino acid sequence of (GGGGS)n (SEQ ID NO: 70), and wherein n is any of 1, 2, 3, 4, 5, or 6. In some embodiments, the peptide linker comprises (or consists essentially of, or consists of) the amino acid sequence of SEQ ID NO: 68 or 69. In some embodiments, the NGF moiety comprises (or consists essentially of, or consists of) the amino acid sequence of SEQ ID NO: 1. In some embodiments, the long-acting NGF polypeptide has a half-life of at least about 10 hours (e.g., at least about any of 11, 12, 13, 14, 15, 16, 17, 18, 19, 20, 21, 22, 23, 24, 25, 30, 35, 40, 45, 50, 55, 60, 70, 80, 90, or 100 hours) when administered to an individual (e.g., human) intravenously, intramuscularly, or subcutaneously. In some embodiments, the long-acting NGF polypeptide causes less pain (e.g., at least about 10%, 20%, 30%, 40%, 50%, 60%, 70%, 80%, 90%, or 100% less pain) compared to an NGF moiety comprising the amino acid sequence of SEQ ID NO: 4.

In some embodiments, the Fc moiety is derived from an IgG1 Fc, such as human IgG1 Fc. In some embodiments, the Fc moiety is a wildtype IgG1 Fc (e.g., human IgG1 Fc) or natural variant thereof. In some embodiments, the Fc moiety is derived from an IgG1 Fc comprising the amino acid sequence of SEQ ID NO: 7 or 8. Thus in some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), and wherein the Fc moiety comprises (or consists essentially of, or consists of) the amino acid sequence of SEQ ID NO: 7 or 8. In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), and wherein the Fc moiety is derived from an IgG1 Fc comprising the amino acid sequence of SEQ ID NO: 7 or 8 (e.g., SEQ ID NO: 8). In some embodiments, the Fc moiety lacks the first 5 amino acids of SEQ ID NO: 7 or 8. In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG1 Fc comprising the amino acid sequence of SEQ ID NO: 7 or 8 (e.g., SEQ ID NO: 8), and wherein the Fc moiety comprises (or consists essentially of, or consists of) a mutation at a position selected from one or more of E233, L234, L235, G236, G237, N297, A327, A330, and P331 relative to SEQ ID NO: 7 or 8 (e.g., SEQ ID NO: 8). In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG1 Fc comprising the amino acid sequence of SEQ ID NO: 7 or 8 (e.g., SEQ ID NO: 8), and wherein the Fc moiety comprises (or consists essentially of, or consists of) a mutation selected from one or more of E233P, L234V, L234A, L235A, L235E, G236del, G237A, N297A, A327G, A330S, and P331S relative to SEQ ID NO: 7 or 8 (e.g., SEQ ID NO: 8). In some embodiments, the Fc moiety further lacks the first 5 amino acids of SEQ ID NO: 7 or 8. In some embodiments, the peptide linker comprises (or consists essentially of, or consists of) the amino acid sequence of (GGGGS)n (SEQ ID NO: 70), and wherein n is any of 1, 2, 3, 4, 5, or 6. In some embodiments, the peptide linker comprises (or consists essentially of, or consists of) the amino acid sequence of SEQ ID NO: 68 or 69. In some embodiments, the NGF moiety comprises (or consists essentially of, or consists of) the amino acid sequence of SEQ ID NO: 1. In some embodiments, the long-acting NGF polypeptide has a half-life of at least about 10 hours (e.g., at least about any of 11, 12, 13, 14, 15, 16, 17, 18, 19, 20, 21, 22, 23, 24, 25, 30, 35, 40, 45, 50, 55, 60, 70, 80, 90, or 100 hours) when administered to an individual (e.g., human) intravenously, intramuscularly, or subcutaneously. In some embodiments, the long-acting NGF polypeptide causes less pain (e.g., at least about 10%, 20%, 30%, 40%, 50%, 60%, 70%, 80%, 90%, or 100% less pain) compared to an NGF moiety comprising the amino acid sequence of SEQ ID NO: 4.

In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG1 Fc comprising the amino acid sequence of SEQ ID NO: 7 or 8 (e.g., SEQ ID NO: 8), and wherein the Fc moiety comprises (or consists essentially of, or consists of) mutations at positions L234, L235, and P331 relative to SEQ ID NO: 7 or 8 (e.g., SEQ ID NO: 8). In some embodiments, the Fc moiety further lacks the first 5 amino acids of SEQ ID NO: 7 or 8 (e.g., SEQ ID NO: 8). In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG1 Fc comprising the amino acid sequence of SEQ ID NO: 7 or 8 (e.g., SEQ ID NO: 8), and wherein the Fc moiety comprises (or consists essentially of, or consists of) L234A, L235A, and P331S mutations relative to SEQ ID NO: 7 or 8 (e.g., SEQ ID NO: 8). In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG1 Fc comprising the amino acid sequence of SEQ ID NO: 7 or 8 (e.g., SEQ ID NO: 8), wherein the Fc moiety comprises (or consists essentially of, or consists of) L234A, L235A, and P331S mutations relative to SEQ ID NO: 7 or 8 (e.g., SEQ ID NO: 8), and wherein the Fc moiety further lacks the first 5 amino acids of SEQ ID NO: 7 or 8 (e.g., SEQ ID NO: 8). Thus in some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), and wherein the Fc moiety comprises (or consists essentially of, or consists of) the amino acid sequence of SEQ ID NO: 11 or 12. In some embodiments, the peptide linker comprises (or consists essentially of, or consists of) the amino acid sequence of (GGGGS)n (SEQ ID NO: 70), and wherein n is any of 1, 2, 3, 4, 5, or 6. In some embodiments, the peptide linker comprises (or consists essentially of, or consists of) the amino acid sequence of SEQ ID NO: 68 or 69. In some embodiments, the NGF moiety comprises (or consists essentially of, or consists of) the amino acid sequence of SEQ ID NO: 1. In some embodiments, there is provided a long-acting NGF polypeptide comprising (or consisting essentially of, or consisting of) the amino acid sequence of any one of SEQ ID NOs: 62-64. In some embodiments, the long-acting NGF polypeptide has a half-life of at least about 10 hours (e.g., at least about any of 11, 12, 13, 14, 15, 16, 17, 18, 19, 20, 21, 22, 23, 24, 25, 30, 35, 40, 45, 50, 55, 60, 70, 80, 90, or 100 hours) when administered to an individual (e.g., human) intravenously, intramuscularly, or subcutaneously. In some embodiments, the long-acting NGF polypeptide causes less pain (e.g., at least about 10%, 20%, 30%, 40%, 50%, 60%, 70%, 80%, 90%, or 100% less pain) compared to an NGF moiety comprising the amino acid sequence of SEQ ID NO: 4.

In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG1 Fc comprising the amino acid sequence of SEQ ID NO: 7 or 8 (e.g., SEQ ID NO: 8), and wherein the Fc moiety comprises (or consists essentially of, or consists of) mutations at positions E233, L234, L235, G236, A327, A330, and P331 relative to SEQ ID NO: 7 or 8 (e.g., SEQ ID NO: 8). In some embodiments, the Fc moiety further lacks the first 5 amino acids of SEQ ID NO: 7 or 8 (e.g., SEQ ID NO: 8). In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG1 Fc comprising the amino acid sequence of SEQ ID NO: 7 or 8 (e.g., SEQ ID NO: 8), and wherein the Fc moiety comprises (or consists essentially of, or consists of) E233P, L234V, L235A, G236del, A327G, A330S, and P331S mutations relative to SEQ ID NO: 7 or 8 (e.g., SEQ ID NO: 8). In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG1 Fc comprising the amino acid sequence of SEQ ID NO: 7 or 8 (e.g., SEQ ID NO: 8), wherein the Fc moiety comprises (or consists essentially of, or consists of) E233P, L234V, L235A, G236del, A327G, A330S, and P331S mutations relative to SEQ ID NO: 7 or 8 (e.g., SEQ ID NO: 8), and wherein the Fc moiety further lacks the first 5 amino acids of SEQ ID NO: 7 or 8 (e.g., SEQ ID NO: 8). Thus in some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), and wherein the Fc moiety comprises (or consists essentially of, or consists of) the amino acid sequence of SEQ ID NO: 15 or 16. In some embodiments, the peptide linker comprises (or consists essentially of, or consists of) the amino acid sequence of (GGGGS)n (SEQ ID NO: 70), and wherein n is any of 1, 2, 3, 4, 5, or 6. In some embodiments, the peptide linker comprises (or consists essentially of, or consists of) the amino acid sequence of SEQ ID NO: 68 or 69. In some embodiments, the NGF moiety comprises (or consists essentially of, or consists of) the amino acid sequence of SEQ ID NO: 1. In some embodiments, there is provided a long-acting NGF polypeptide comprising (or consisting essentially of, or consisting of) the amino acid sequence of SEQ ID NO: 66. In some embodiments, the long-acting NGF polypeptide has a half-life of at least about 10 hours (e.g., at least about any of 11, 12, 13, 14, 15, 16, 17, 18, 19, 20, 21, 22, 23, 24, 25, 30, 35, 40, 45, 50, 55, 60, 70, 80, 90, or 100 hours) when administered to an individual (e.g., human) intravenously, intramuscularly, or subcutaneously. In some embodiments, the long-acting NGF polypeptide causes less pain (e.g., at least about 10%, 20%, 30%, 40%, 50%, 60%, 70%, 80%, 90%, or 100% less pain) compared to an NGF moiety comprising the amino acid sequence of SEQ ID NO: 4.

In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG1 Fc comprising the amino acid sequence of SEQ ID NO: 7 or 8 (e.g., SEQ ID NO: 8), and wherein the Fc moiety comprises (or consists essentially of, or consists of) mutations at positions L234, L235, G237, A330, and P331 relative to SEQ ID NO: 7 or 8 (e.g., SEQ ID NO: 8). In some embodiments, the Fc moiety further lacks the first 5 amino acids of SEQ ID NO: 7 or 8 (e.g., SEQ ID NO: 8). In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOS: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG1 Fc comprising the amino acid sequence of SEQ ID NO: 7 or 8 (e.g., SEQ ID NO: 8), and wherein the Fc moiety comprises (or consists essentially of, or consists of) L234A, L235E, G237A, A330S, and P331S mutations relative to SEQ ID NO: 7 or 8 (e.g., SEQ ID NO: 8). In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG1 Fc comprising the amino acid sequence of SEQ ID NO: 7 or 8 (e.g., SEQ ID NO: 8), wherein the Fc moiety comprises (or consists essentially of, or consists of) L234A, L235E, G237A, A330S, and P331S mutations relative to SEQ ID NO: 7 or 8 (e.g., SEQ ID NO: 8), and wherein the Fc moiety further lacks the first 5 amino acids of SEQ ID NO: 7 or 8 (e.g., SEQ ID NO: 8). Thus in some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), and wherein the Fc moiety comprises (or consists essentially of, or consists of) the amino acid sequence of SEQ ID NO: 13 or 14. In some embodiments, the peptide linker comprises (or consists essentially of, or consists of) the amino acid sequence of (GGGGS)n (SEQ ID NO: 70), and wherein n is any of 1, 2, 3, 4, 5, or 6. In some embodiments, the peptide linker comprises (or consists essentially of, or consists of) the amino acid sequence of SEQ ID NO: 68 or 69. In some embodiments, the NGF moiety comprises (or consists essentially of, or consists of) the amino acid sequence of SEQ ID NO: 1. In some embodiments, there is provided a long-acting NGF polypeptide comprising (or consisting essentially of, or consisting of) the amino acid sequence of SEQ ID NO: 65. In some embodiments, the long-acting NGF polypeptide has a half-life of at least about 10 hours (e.g., at least about any of 11, 12, 13, 14, 15, 16, 17, 18, 19, 20, 21, 22, 23, 24, 25, 30, 35, 40, 45, 50, 55, 60, 70, 80, 90, or 100 hours) when administered to an individual (e.g., human) intravenously, intramuscularly, or subcutaneously. In some embodiments, the long-acting NGF polypeptide causes less pain (e.g., at least about 10%, 20%, 30%, 40%, 50%, 60%, 70%, 80%, 90%, or 100% less pain) compared to an NGF moiety comprising the amino acid sequence of SEQ ID NO: 4.

In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG1 Fc comprising the amino acid sequence of SEQ ID NO: 7 or 8 (e.g., SEQ ID NO: 8), and wherein the Fc moiety comprises (or consists essentially of, or consists of) a mutation at position N297 relative to SEQ ID NO: 7 or 8 (e.g., SEQ ID NO: 8). In some embodiments, the Fc moiety further lacks the first 5 amino acids of SEQ ID NO: 7 or 8 (e.g., SEQ ID NO: 8). In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG1 Fc comprising the amino acid sequence of SEQ ID NO: 7 or 8 (e.g., SEQ ID NO: 8), and wherein the Fc moiety comprises (or consists essentially of, or consists of) an N297A mutation relative to SEQ ID NO: 7 or 8 (e.g., SEQ ID NO: 8). In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG1 Fc comprising the amino acid sequence of SEQ ID NO: 7 or 8 (e.g., SEQ ID NO: 8), wherein the Fc moiety comprises (or consists essentially of, or consists of) an N297A mutation relative to SEQ ID NO: 7 or 8 (e.g., SEQ ID NO: 8), and wherein the Fc moiety further lacks the first 5 amino acids of SEQ ID NO: 7 or 8 (e.g., SEQ ID NO: 8). Thus in some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), and wherein the Fc moiety comprises (or consists essentially of, or consists of) the amino acid sequence of SEQ ID NO: 9 or 10. In some embodiments, the peptide linker comprises (or consists essentially of, or consists of) the amino acid sequence of (GGGGS)n (SEQ ID NO: 70), and wherein n is any of 1, 2, 3, 4, 5, or 6. In some embodiments, the peptide linker comprises (or consists essentially of, or consists of) the amino acid sequence of SEQ ID NO: 68 or 69. In some embodiments, the NGF moiety comprises (or consists essentially of, or consists of) the amino acid sequence of SEQ ID NO: 1. In some embodiments, there is provided a long-acting NGF polypeptide comprising (or consisting essentially of, or consisting of) the amino acid sequence of SEQ ID NO: 61. In some embodiments, the long-acting NGF polypeptide has a half-life of at least about 10 hours (e.g., at least about any of 11, 12, 13, 14, 15, 16, 17, 18, 19, 20, 21, 22, 23, 24, 25, 30, 35, 40, 45, 50, 55, 60, 70, 80, 90, or 100 hours) when administered to an individual (e.g., human) intravenously, intramuscularly, or subcutaneously. In some embodiments, the long-acting NGF polypeptide causes less pain (e.g., at least about 10%, 20%, 30%, 40%, 50%, 60%, 70%, 80%, 90%, or 100% less pain) compared to an NGF moiety comprising the amino acid sequence of SEQ ID NO: 4.

In some embodiments, the Fc moiety is derived from an IgG4 Fc, such as human IgG4 Fc. In some embodiments, the Fc moiety is a wildtype IgG4 Fc (e.g., human IgG4 Fc) or natural variant thereof. In some embodiments, the Fc moiety is derived from an IgG4 Fc comprising the amino acid sequence of SEQ ID NO: 17. Thus in some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), and wherein the Fc moiety comprises (or consists essentially of, or consists of) the amino acid sequence of SEQ ID NO: 17. In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), and wherein the Fc moiety is derived from an IgG4 Fc comprising the amino acid sequence of SEQ ID NO: 17. In some embodiments, the Fc moiety lacks the first 5 amino acids of SEQ ID NO: 17. In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG4 Fc comprising the amino acid sequence of SEQ ID NO: 17, and wherein the Fc moiety comprises (or consists essentially of, or consists of) a mutation at a position selected from one or more of S228, F234, and L235 relative to SEQ ID NO: 17. In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG4 Fc comprising the amino acid sequence of SEQ ID NO: 17, and wherein the Fc moiety comprises (or consists essentially of, or consists of) mutations at positions S228, F234, and L235 relative to SEQ ID NO: 17. In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG4 Fc comprising the amino acid sequence of SEQ ID NO: 17, and wherein the Fc moiety comprises (or consists essentially of, or consists of) a mutation selected from one or more of S228P, F234A, and L235A relative to SEQ ID NO: 17. In some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), wherein the Fc moiety is derived from an IgG4 Fc comprising the amino acid sequence of SEQ ID NO: 17, and wherein the Fc moiety comprises (or consists essentially of, or consists of) S228P, F234A, and L235A mutations relative to SEQ ID NO: 17. In some embodiments, the Fc moiety further lacks the first 5 amino acids of SEQ ID NO: 17. Thus in some embodiments, there is provided a long-acting NGF polypeptide comprising from N-terminus to C-terminus an NGF moiety, an optional peptide linker (e.g., any one of SEQ ID NOs: 68-99, such as any one of SEQ ID NOs: 68-72), and an Fc moiety, wherein the NGF moiety comprises (or consists essentially of, or consists of) the amino acid sequence of any one of SEQ ID NOs: 1-4 (e.g., any of SEQ ID NOs: 1-3), and wherein the Fc moiety comprises (or consists essentially of, or consists of) the amino acid sequence of SEQ ID NO: 18. In some embodiments, the peptide linker comprises (or consists essentially of, or consists of) the amino acid sequence of (GGGGS)n (SEQ ID NO: 70), and wherein n is any of 1, 2, 3, 4, 5, or 6. In some embodiments, the peptide linker comprises (or consists essentially of, or consists of) the amino acid sequence of SEQ ID NO: 68 or 69. In some embodiments, the NGF moiety comprises (or consists essentially of, or consists of) the amino acid sequence of SEQ ID NO: 1. In some embodiments, there is provided a long-acting NGF polypeptide comprising (or consisting essentially of, or consisting of) the amino acid sequence of SEQ ID NO: 67. In some embodiments, the long-acting NGF polypeptide has a half-life of at least about 10 hours (e.g., at least about any of 11, 12, 13, 14, 15, 16, 17, 18, 19, 20, 21, 22, 23, 24, 25, 30, 35, 40, 45, 50, 55, 60, 70, 80, 90, or 100 hours) when administered to an individual (e.g., human) intravenously, intramuscularly, or subcutaneously. In some embodiments, the long-acting NGF polypeptide causes less pain (e.g., at least about 10%, 20%, 30%, 40%, 50%, 60%, 70%, 80%, 90%, or 100% less pain) compared to an NGF moiety comprising the amino acid sequence of SEQ ID NO: 4.

In some embodiments, there is provided a long-acting NGF polypeptide comprising (or consisting essentially of, or consisting of) the amino acid sequence of any of SEQ ID NOS: 34, 36, 38, 40, 42, 44, and 46. In some embodiments, there is provided a long-acting NGF polypeptide comprising (or consisting essentially of, or consisting of) the amino acid sequence of any of SEQ ID NOs: 34, 36, 38, 40, 42, 44, and 46, excluding the signal peptide sequence of SEQ ID NO: 6. In some embodiments, the long-acting NGF polypeptide has a half-life of at least about 10 hours (e.g., at least about any of 11, 12, 13, 14, 15, 16, 17, 18, 19, 20, 21, 22, 23, 24, 25, 30, 35, 40, 45, 50, 55, 60, 70, 80, 90, or 100 hours) when administered to an individual (e.g., human) intravenously, intramuscularly, or subcutaneously. In some embodiments, the long-acting NGF polypeptide causes less pain (e.g., at least about 10%, 20%, 30%, 40%, 50%, 60%, 70%, 80%, 90%, or 100% less pain) compared to an NGF moiety comprising the amino acid sequence of SEQ ID NO: 4.

### NGF Moieties

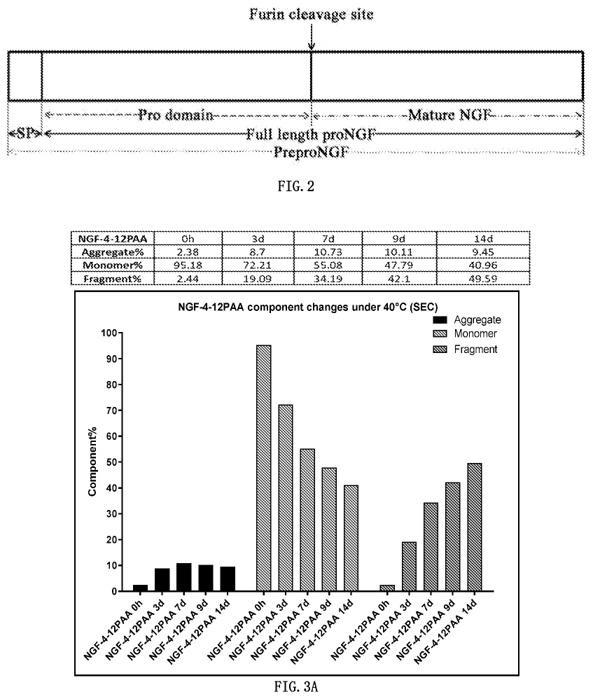

NGF is initially in a 7S, 130 kDa complex of 3 proteins—α-NGF, β-NGF, and γ-NGF (2:1:2 ratio) when expressed. The γ subunit of this complex acts as a serine protease, and cleaves the N-terminal of the β subunit, thereby activating the protein into functional NGF. The human NGF gene is located on a short arm of chromosome 1, and the complete NGF exon encodes 241 amino acids, commonly referred to as a preproNGF precursor (SEQ ID NO: 50). PreproNGF precursor contains a signal peptide sequence (SEQ ID NO: 6), a propeptide (SEQ ID NO: 5), and the mature NGF sequence (β-NGF, SEQ ID NO: 4). The signal peptide of the preproNGF precursor is cleaved in the endoplasmic reticulum to form a proNGF precursor (223 amino acids; SEQ ID NO: 54). The proNGF precursor exists in a form of a homodimer in the endoplasmic reticulum, and is then transferred to a Golgi apparatus, in which the proNGF precursor dimer is cleaved by Furin at 3 Furin motifs on the propeptide. Cleavage by Furin at the Furin motif located at positions-1 and -2 with respect to the mature NGF sequence generates mature β-NGF dimer, each monomer contains 118 or 120 amino acids. Mature β-NGF dimer is then transported outside of the cell. Some uncleaved proNGF precursors are also secreted outside of the cell. See FIG. 2 for NGF structure.

NGF is present in a variety of species and is abundant in male mouse submandibular gland, bovine seminal plasma, snake venom, guinea pig prostate, human placental tissue, etc. The amino acid sequence homology between mouse NGF and human NGF is up to about 90%.

NGF binds with the tropomyosin receptor kinase A (TrkA) and low-affinity NGF receptor (LNGFR/p75NTR), both associated with neurodegenerative disorders. NGF binding to TrkA receptor drives the homodimerization of the receptor, which in turn causes the autophosphorylation of the tyrosine kinase segment, leading to the activation of PI 3-kinase, ras, and PLC signaling pathways. The p75NTR receptor can also form a heterodimer with TrkA, which has higher affinity and specificity for NGF. On the other hand, proNGF binds with high affinity to p75NTR and to sortilin, resulting in a signaling complex that recruits NRAGE and Rac to activate the JNK signaling cascades, which primarily drive apoptosis. ProNGF binding to p75NTR can also promote the activation of NFκB to induce neuronal survival.

As used herein, the term “NGF moiety” refers to an NGF molecule, a species variant, fragment, mutant, or derivative thereof. The NGF moiety can be truncated versions, post-translationally modified versions, hybrid variants, peptide mimetics, biologically active fragments, deletion variants, substitution variants, or addition variants that maintain at least some degree of the parental NGF activity, such as binding to the NGF receptor and inducing a signal transmission through the NGF receptor. “Parental NGF” or “parent NGF” described herein refers to the NGF reference sequence from which the NGF moiety is engineered, modified, or derived from.

In some embodiments, the NGF moiety is a wildtype NGF. In some embodiments, the NGF moiety is an NGF natural variant. In some embodiments, the NGF moiety is an analog of an NGF, such as an NGF comprising no more than about 6 amino acids (such as 6, 5, 4, 3, 2, or 1 aa) mutation sites. In some embodiments, the NGF moiety is a derivative of an NGF. As used herein, the term “a derivative of an NGF” refers to a molecule having an amino acid sequence of an NGF or an analog of the NGF, but also having an additional chemical modification at one or more of the amino acid side groups, a carbon atoms, terminal amino groups, or terminal carboxyl groups. The chemical modifications include, but are not limited to, adding chemical moieties, creating new bonds, and removing chemical moieties. The modification at amino acid side groups includes, but is not limited to, an acylation of an epsilon amino group of lysine, an N-alkylation of arginine, histidine or lysine, an alkylation of carboxyl of glutamic acid or aspartic acid, and a deamination of glutamine or asparagine. The modification at terminal amino groups includes, but is not limited to, deamination, N-lower alkyl, N-di-lower alkyl, and N-acyl modifications. The modification at terminal carboxyl groups includes, but is not limited to, amide, lower alkyl acyl, dialkyl amide, and lower alkyl ester modifications. In some embodiments, the lower alkyl group is a C1-C4 alkyl group. In addition, one or more side groups or terminal groups may be protected by a protecting group known to a person skilled in the field of chemistry. An alpha carbon of an amino acid may be mono- or di-methylated. In some embodiments, the NGF moiety is a modified NGF, such as a pegylated NGF, or covalently modified NGF, such as glycosylated NGF.

The NGF moiety can be derived from any organism, such as mammals, including, but are not limited to, livestock animals (e.g., cows, sheep, goats, cats, dogs, donkeys, and horses), primates (e.g., human and non-human primates such as monkeys or chimpanzees), rabbits, and rodents (e.g., mice, rats, gerbils, and hamsters).

In some embodiments, the NGF moiety is a human NGF (hNGF). In some embodiments, the NGF moiety is a wildtype (wt) hNGF. In some embodiments, the NGF moiety is an hNGF natural variant. In some embodiments, the NGF moiety is an analog of an hNGF, such as an hNGF comprising no more than about 6 amino acids (such as 6, 5, 4, 3, 2, or 1 aa) mutation sites. In some embodiments, the NGF moiety is a derivative of an hNGF. Many active fragments, analogs, and derivatives of hNGF are well known in the art, and any one of these active fragments, analogs, and derivatives may be an NGF moiety used in the present application.

The NGF moiety described herein can be an NGF isolated from a variety of sources, such as from human tissues or from another source, or prepared by recombinant or synthetic methods. In some embodiments, the NGF moiety is a recombinant NGF, such as a recombinant hNGF (rhNGF). In some embodiments, the NGF moiety is a murine NGF, such as recombinant murine NGF.

In some embodiments, the NGF moiety is a full-length NGF. In some embodiments, the NGF moiety is a functional fragment of NGF that is capable of producing most or full biological activity of a full-length NGF molecule, such as most or full biological activity of a full-length β-NGF. In some embodiments, the NGF moiety is a preproNGF (such as human preproNGF) or an active fragment thereof, i.e., comprising full length or fragments of all of signal peptide, propeptide, and β-NGF. In some embodiments, the NGF moiety comprises an amino acid sequence of any one of SEQ ID NOs: 47-50. In some embodiments, the NGF moiety is a proNGF (such as human proNGF) or an active fragment thereof, i.e., comprising full length or fragments of both propeptide and β-NGF. In some embodiments, the NGF moiety comprises an amino acid sequence of any one of SEQ ID NOs: 51-54. In some embodiments, the NGF moiety is a mature NGF or an active fragment thereof, i.e., comprising full length or active fragment of β-NGF (such as human β-NGF). In some embodiments, the NGF moiety comprises an amino acid sequence of any one of SEQ ID NOs: 1-4. In some embodiments, the NGF moiety is a wildtype human β-NGF (SEQ ID NO: 4). In some embodiments, the NGF moiety is a wildtype human β-NGF with the last 2 amino acids truncated (SEQ ID NO: 3). In some embodiments, the NGF moiety comprises a signal peptide at the N-terminus of β-NGF, the signal peptide is either from a different molecule or from the same NGF molecule. In some embodiments, the signal peptide comprises the amino acid sequence of SEQ ID NO: 6. In some embodiments, the NGF moiety comprises a propeptide at the N-terminus of β-NGF. In some embodiments, the propeptide comprises the amino acid sequence of SEQ ID NO: 5.

In some embodiments, the NGF moiety is a mutant or variant NGF, such as a mutant or variant NGF capable of producing most or full biological activity of a wildtype NGF. The mutant NGF moiety can include a mutation in one or more amino acids of the NGF molecule (e.g., mature β-NGF). In some embodiments, the mutant NGF moiety includes an amino acid substitution at one or more amino acid positions in the NGF. In some embodiments, the mutant NGF moiety includes deletions or insertions of amino acids at one or more amino acid positions in the NGF. In some embodiments, the mutant NGF moiety includes modifications of one or more amino acids in the NGF.

In some embodiments, the NGF moiety has one or more conservative amino acid substitutions. “Conservative substitution” refers to the substitution of another amino acid with the same net charge and approximately the same size and shape as the substituted amino acid. When the total number of carbon atoms and heteroatoms on their side chains differ by no more than 4, amino acids with aliphatic or substituted aliphatic amino acid side chains are roughly the same size. When the number of branches on their side chains does not differ by more than one, amino acids have roughly the same shape. Amino acids having a phenyl or substituted phenyl group on the side chain can be considered to be approximately the same in size and shape. Unless otherwise specified, natural amino acids are preferably used for conservative substitutions. Also see “Amino acid substituents” subsection below.

“Amino acid” is used herein in its broadest sense, including both naturally occurring amino acids and non-naturally occurring amino acids, including amino acid analogs and derivatives. The latter includes molecules that contain amino acid moieties. Those skilled in the art will realize that according to this broad definition, amino acids herein include, for example, naturally occurring L-amino acids that form proteins; D-amino acids; chemically modified amino acids, such as amino acid analogs and derivatives; naturally occurring amino acids that do not form protein, such as norleucine, β-alanine, ornithine, GABA, etc.; and chemically synthesized compounds with amino acid characteristics known in the art. The term “protein-forming” as used herein refers to amino acids that can be incorporated into peptides, polypeptides or proteins of cells through metabolic pathways.

Insertion of non-naturally occurring amino acids, including synthetic non-natural amino acids, substituted amino acids, or one or more D-amino acids, into the long-acting NGF polypeptides (or NGF moiety) of the present invention can have multiple benefits. D-amino acid-containing peptides and the like exhibit increased stability in vitro or in vivo compared to their counterparts containing L-amino acid. Therefore, when greater intracellular stability is desired, the construction of peptides, such as by incorporation of D-amino acids, is particularly useful. Particularly, D-peptide and the like are resistant to endogenous peptidase and protease activity, thereby improving the bioavailability of the molecule and extending the lifespan in vivo when needed. In addition, D-peptide and the like cannot be effectively processed for limited presentation by type II major histocompatibility complexes (MHC) to T helper cells, so less prone to induce humoral immune responses in the subject.

In some embodiments, the NGF moiety is a mutant or variant NGF that has reduced side effect (e.g., pain) compared to the wildtype NGF, or is painless. In some embodiments, the NGF moiety is a mutant or variant NGF that reduces at least about 5% (such as at least about any of 10%, 20%, 30%, 40%, 50%, 60%, 70%, 80%, 90%, 95%, or 100%) pain compared to that of a wildtype NGF, such as reduces at least about 5% pain at one or more (e.g., all) time points post administration. In some embodiments, the NGF moiety is a mutant or variant NGF that increases at least about 5% (such as at least about any of 10%, 20%, 30%, 40%, 50%, 60%, 70%, 80%, 90%, 95%, or 100%) pain threshold compared to that of a wildtype NGF. For example, in some embodiments, the pain threshold of an individual is about 8, the pain threshold drops to about 6 after administration with a wildtype NGF, while the pain threshold stays at around 8 after administration with a mutant or variant NGF (or long-acting NGF polypeptide comprising thereof described herein), i.e., about 25% reduction in pain, or about 25% increase in pain threshold. In some embodiments, the NGF moiety is a mutant or variant NGF as described in CN107286233A, WO2017157325, and WO2017157326, the contents of which are incorporated herein by reference in their entirety. In some embodiments, the NGF moiety comprises an F12E mutation relative to human wildtype β-NGF sequence (SEQ ID NO: 4). In some embodiments, the NGF moiety comprises an amino acid sequence of SEQ ID NO: 2. In some embodiments, the NGF moiety comprises an F12E mutation and the last 2 amino acids truncated relative to human wildtype β-NGF sequence (SEQ ID NO: 4). In some embodiments, the NGF moiety comprises an amino acid sequence of SEQ ID NO: 1 (hereinafter also referred to as “mNGF118”).

Amino acid sequence variants of an NGF moiety or a long-acting NGF polypeptide described herein may be prepared by introducing appropriate modifications into the nucleic acid sequence encoding the protein, or by peptide synthesis. Such modifications include, for example, deletions from, and/or insertions into and/or substitutions of residues within the amino acid sequences of the NGF moiety or long-acting NGF polypeptide. Any combination of deletion, insertion, and substitution can be made to arrive at the final construct, provided that the final construct possesses the desired characteristics, e.g., retained/improved ligand-receptor binding, retained/enhanced bioactivity (e.g., promoting growth, maintenance, proliferation, and/or survival of neurons), retained/enhanced half-life, retained/reduced ADCC/CDC, retained/reduced pain-causing activity, etc.

Conservative substitutions are shown in Table A. More substantial changes are provided in Table A under the heading of “exemplary substitutions,” and as further described below in reference to amino acid side chain classes. Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. Non-conservative substitutions will entail exchanging a member of one of these classes for another class. Amino acid substitutions may be introduced into the protein constructs and the products screened for a desired activity mentioned above.

TABLE A
Amino acid substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; | Gln |
| | Arg | |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; | Tyr |
| | Ala; Tyr | |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

### Fc Moieties

The long-acting NGF polypeptide descried herein comprises an Fc moiety at the C-terminus.

In some embodiments, the Fc moiety is derived from any of IgA, IgD, IgE, IgG, and IgM, and subtypes thereof. IgG has the highest serum content and longest serum half-life among all immunoglobulins. Unlike other immunoglobulins, IgG is effectively recycled after binding to Fc receptors (FcRs). In some embodiments, the Fc moiety is derived from an IgG (e.g., IgG1, IgG2, IgG3, or IgG4). In some embodiments, the Fc moiety is derived from a human IgG. In some embodiments, the Fc moiety comprises CH2 and CH3. In some embodiments, the Fc moiety further comprises full or part of the hinge region. In some embodiments, the Fc moiety is derived from a human IgG1 or human IgG4. In some embodiments, the two subunits of the Fc moiety dimerize via one or more (e.g., 1, 2, 3, 4, or more) disulfide bonds. In some embodiments, each subunit of the Fc moiety comprises a full-length Fc sequence. In some embodiments, each subunit of the Fc moiety comprises an N-terminus truncated Fc sequence, such as truncated Fc domain with less N-terminal cysteines in order to reduce disulfide bond mis-pairing during dimerization. In some embodiments, the Fc moiety is truncated at the N-terminus, e.g., lacks the first 1, 2, 3, 4, 5, 6, 7, 8, 9, or 10 amino acids of a complete immunoglobulin Fc domain. In some embodiments, the Fc moiety contains one or more mutations, such as insertion, deletion, and/or substitution.

It is desired to screen for Fc moieties that provide long-acting NGF polypeptides described herein with high biological activity, long half-life, and low immunotoxicity (e.g., ADCC and/or CDC).

Via the Fc domain, Fc-containing protein can activate complement and interact with Fc receptors (FcRs). This inherent immunoglobulin feature has been viewed unfavorably because NGF-Fc fusion proteins may be targeted to cells expressing Fc receptors rather than the preferred NGF receptor expressing cells, and in further view of the long half-life of Fc fusion proteins, making their application in a therapeutic setting difficult due to systemic toxicity. Thus in some embodiments, the Fc moiety is engineered (e.g., comprises one or more amino acid mutations) to have altered binding to an FcR, specifically altered binding to an Fcγ receptor (responsible for ADCC), and/or altered effector function, such as altered antibody-dependent cell-mediated cytotoxicity (ADCC), Antibody-Dependent Cellular Phagocytosis (ADCP), and/or Complement-Dependent Cytotoxicity (CDC). Preferably, such amino acid mutation(s) does not reduce binding to FcRn receptors (responsible for half-life).

Fc moiety (e.g., human IgG1 Fc) mutated to remove one or more effector functions such as ADCC, ADCP, or CDC, is hereinafter referred to as “effectorless” or “almost effectorless” Fc moiety. For example, in some embodiments, the Fc moiety is an effectorless human IgG1 Fc comprising one or more of the following mutations (such as in each of Fc subunits): L234A, L235E, G237A, A330S, and P331S. The combinations of K322A, L234A, and L235A in IgG1 Fc are sufficient to almost completely abolish FcγR and Clq binding (Hezareh et al. J Virol 75, 12161-12168, 2001). MedImmune identified that a set of three mutations L234F/L235E/P331S have a very similar effect (Oganesyan et al., Acta Crystallographica 64, 700-704, 2008). In some embodiments, the Fc moiety comprises a modification of the glycosylation on N297 of the IgG1 Fc domain, which is known to be required for optimal FcR interaction. The Fc moiety modification can be any suitable IgG Fc engineering mentioned in Wang et al. (“IgG Fc engineering to modulate antibody effector functions,” Protein Cell. 2018 January; 9 (1): 63-73), the content of which is incorporated herein by reference in its entirety.

In some embodiments, the long-acting NGF polypeptides described herein have no ADCC and/or CDC, or no detectable ADCC and/or CDC. In some embodiments, the long-acting NGF polypeptides described herein have at least about 5% (such as at least about any of 10%, 20%, 30%, 40%, 50%, 60%, 70%, 80%, 90%, 95% or 100%) reduction in ADCC and/or CDC compared to an NGF-Fc construct comprising the same NGF moiety but a wild type or unmodified Fc fragment.

### Glycosylation Variants

In some embodiments, the Fc moiety or long-acting NGF polypeptide is altered to increase or decrease the extent to which the construct is glycosylated. Addition or deletion of glycosylation sites to an Fc moiety may be conveniently accomplished by altering the amino acid sequence such that one or more glycosylation sites is created or removed.

Native Fc-containing proteins produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an Fc moiety may be made in order to create certain improved properties.

In some embodiments, the Fc moiety or long-acting NGF polypeptide described herein is provided having a carbohydrate structure that lacks fucose attached (directly or indirectly) to the Fc moiety. For example, the amount of fucose in such Fc moiety or long-acting NGF polypeptide may be from about 1% to about 80%, from about 1% to about 65%, from about 5% to about 65%, or from about 20% to about 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc domain (EU numbering of Fc region residues); however, Asn297 may also be located about +3 amino acids upstream or downstream of position 297, i.e., between positions 294 and 300, due to minor sequence variations in Fc domains. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87:614 (2004). Examples of cell lines capable of producing defucosylated Fc-containing proteins include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87:614 (2004); Kanda, Y. et al., Biotechnol. Bioeng., 94 (4): 680-688 (2006); and WO2003/085107).

### Effector Function Variants

In some embodiments, the present application contemplates an Fc moiety or long-acting NGF polypeptide that possesses some but not all Fc effector functions, which makes it a desirable candidate for applications in which the half-life of the long-acting NGF polypeptide in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the Fc moiety or long-acting NGF polypeptide lacks FcγR binding (hence likely lacking ADCC activity), but retains FcRn binding ability. The primary cells for mediating ADCC, Natural Killer (NK) cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the long-acting NGF polypeptide is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18 (12): 1759-1769 (2006)).

Fc moieties with reduced effector function include those with substitution of one or more of Fc region residues 238, 265, 269, 270, 297, 327 and 329 (U.S. Pat. No. 6,737,056). Such Fc mutants include substitutions at two or more of amino acid positions 265, 269, 270, 297 and 327, including the so-called “DANA” Fc mutant with substitution of residues 265 and 297 to alanine (U.S. Pat. No. 7,332,581). Certain antibody variants with improved or diminished binding to FcRs are described (see, e.g., U.S. Pat. No. 6,737,056; WO 2004/056312, and Shields et al., J. Biol. Chem. 9 (2): 6591-6604 (2001)). In some embodiments, alterations are made in the Fc domain that result in altered (i.e., either improved or diminished) C1q binding and/or CDC, e.g., as described in U.S. Pat. No. 6,194,551, WO 99/51642, and Idusogie et al. J. Immunol. 164:4178-4184 (2000).

In some embodiments, the Fc moiety comprises one or more amino acid substitutions, which increase half-life and/or improve binding to the neonatal Fc receptor (FcRn). Antibodies with increased half-lives and improved binding to the neonatal FcRn, which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc domain with one or more substitutions therein which improve binding of the Fc region to FcRn. Such Fc variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc domain variants.

### Cysteine-Engineered Variants

In some embodiments, it may be desirable to create cysteine-engineered Fc moieties or long-acting NGF polypeptides, in which one or more residues of an Fc domain are substituted with cysteine residues. In some embodiments, the substituted residues occur at accessible sites of the Fc moiety or long-acting NGF polypeptide. By substituting those residues with cysteine, reactive thiol groups are thereby positioned at accessible sites of the Fc moiety or long-acting NGF polypeptide and may be used to conjugate the molecule to other moieties, such as drug moieties or linker-drug moieties, to create a long-acting NGF polypeptide-conjugate. In some embodiments, any one or more of the following residues may be substituted with cysteine: A118 (EU numbering) of the heavy chain; and $400 (EU numbering) of the heavy chain Fc domain. Cysteine engineered molecules may be generated as described, e.g., in U.S. Pat. No. 7,521,541.

In some embodiments, the Fc moiety is derived from an IgG1 Fc. In some embodiments, the Fc moiety is derived from a human IgG1 Fc. In some embodiments, the Fc moiety is a wildtype IgG1 Fc (IGHG1*05). In some embodiments, the Fc moiety comprises (or consists essentially of, or consists of) the amino acid sequence of SEQ ID NO: 7. In some embodiments, the Fc moiety is a natural variant of IgG1 (e.g., IGHG1*03, which comprises D239E and L241M double mutations relative to IGHG1*05). In some embodiments, the Fc moiety comprises (or consists essentially of, or consists of) the amino acid sequence of SEQ ID NO: 8. In some embodiments, the Fc moiety does not comprise the hinge region of an IgG1 Fc. In some embodiments, the Fc moiety comprises at most about 5 amino acids truncated from the N-terminus of an IgG1 Fc, such as truncating the first, the first two, the first three, the first four, or the first five amino acids from the N-terminus of the IgG1 Fc. In some embodiments, the Fc moiety comprises one or more effectorless mutations and/or deglycosylation mutation(s). In some embodiments, the Fc moiety comprises (or consists essentially of, or consists of) a mutation at a position selected from one or more of E233, L234, L235, G236, G237, N297, A327, A330, and P331 relative to SEQ ID NO: 7 or 8. In some embodiments, the Fc moiety comprises a mutation selected from one or more of E233P, L234V, L234A, L235A, L235E, G236del, G237A, N297A, A327G, A330S, and P331S relative to SEQ ID NO: 7 or 8. In some embodiments, the Fc moiety further lacks the first (N-terminus) 5 amino acids of SEQ ID NO: 7 or 8. In some embodiments, the Fc moiety comprises (or consists essentially of, or consists of) a mutation at position N297 relative to SEQ ID NO: 7 or 8. In some embodiments, the Fc moiety comprises (or consists essentially of, or consists of) an N297A mutation relative to SEQ ID NO: 7 or 8. In some embodiments, the Fc moiety comprises (or consists essentially of, or consists of) the amino acid sequence of SEQ ID NO: 9 or 10. In some embodiments, the Fc moiety comprises (or consists essentially of, or consists of) mutations at positions L234, L235, and P331 relative to SEQ ID NO: 7 or 8. In some embodiments, the Fc moiety comprises (or consists essentially of, or consists of) L234A, L235A, and P331S mutations relative to SEQ ID NO: 7 or 8. In some embodiments, the Fc moiety comprises (or consists essentially of, or consists of) the amino acid sequence of SEQ ID NO: 11 or 12. In some embodiments, the Fc moiety comprises (or consists essentially of, or consists of) mutations at positions L234, L235, G237, A330, and P331 relative to SEQ ID NO: 7 or 8. In some embodiments, the Fc moiety comprises (or consists essentially of, or consists of) L234A, L235E, G237A, A330S, and P331S mutations relative to SEQ ID NO: 7 or 8. In some embodiments, the Fc moiety comprises (or consists essentially of, or consists of) the amino acid sequence of SEQ ID NO: 13 or 14. In some embodiments, the Fc moiety comprises (or consists essentially of, or consists of) mutations at positions E233, L234, L235, G236, A327, A330, and P331 relative to SEQ ID NO: 7 or 8. In some embodiments, the Fc moiety comprises (or consists essentially of, or consists of) E233P, L234V, L235A, G236del, A327G, A330S, and P331S mutations relative to SEQ ID NO: 7 or 8. In some embodiments, the Fc moiety comprises (or consists essentially of, or consists of) the amino acid sequence of SEQ ID NO: 15 or 16.

In some embodiments, the Fc moiety is derived from an IgG4 Fc. In some embodiments, the Fc moiety is derived from a human IgG4 Fc. In some embodiments, the Fc moiety is a wildtype IgG4 Fc. In some embodiments, the Fc moiety comprises (or consists essentially of, or consists of) the amino acid sequence of SEQ ID NO: 17. In some embodiments, the Fc moiety is a natural variant of IgG4. In some embodiments, the Fc moiety does not comprise the hinge region of an IgG4 Fc. In some embodiments, the Fc moiety comprises at most about 5 amino acids truncated from the N-terminus of an IgG4 Fc, such as truncating the first, the first two, the first three, the first four, or the first five amino acids from the N-terminus of the IgG4 Fc. In some embodiments, the Fc moiety comprises one or more effectorless mutations and/or deglycosylation mutation(s). In some embodiments, the Fc moiety comprises (or consists essentially of, or consists of) a mutation at a position selected from one or more of S228, F234, and L235 relative to SEQ ID NO: 17. In some embodiments, the Fc moiety comprises (or consists essentially of, or consists of) mutations at positions S228, F234, and L235 relative to SEQ ID NO: 17. In some embodiments, the Fc moiety comprises (or consists essentially of, or consists of) a mutation selected from one or more of S228P, F234A, and L235A relative to SEQ ID NO: 17. In some embodiments, the Fc moiety comprises (or consists essentially of, or consists of) mutations S228P, F234A, and L235A relative to SEQ ID NO: 17. In some embodiments, the Fc moiety comprises (or consists essentially of, or consists of) the amino acid sequence of SEQ ID NO: 18. In some embodiments, the Fc moiety further lacks the first (N-terminus) 5 amino acids of SEQ ID NO: 17. In some embodiments, the Fc moiety comprises (or consists essentially of, or consists of) the amino acid sequence of SEQ ID NO: 19 or 20.

### Linkers

The NGF moiety and the Fc moiety are connected via an optional linker (e.g., peptide linker, non-peptide linker). In some embodiments, the linker is a flexible linker. In some embodiments, the linker is a stable linker. In general, a desirable linker does not affect or significantly affect the proper fold and conformation formed by the configuration of the long-acting NGF polypeptide described herein. Preferably, the linker confers flexibility to the long-acting NGF polypeptide, retains/improves NGF biological function, and/or does not significantly affect in vivo half-life and/or stability of the long-acting NGF polypeptide. In some embodiments, the linker is a stable linker (e.g., not cleavable by protease, especially MMPs).

In some embodiments, the linker is a peptide linker. The peptide linker can be of any length. In some embodiments, the peptide linker is from about 1 to about 10 amino acids long, from about 3 to about 18 amino acids long, from about 1 to about 20 amino acids long, from about 10 to about 20 amino acids long, from about 21 to about 30 amino acids long, from about 1 to about 30 amino acids long, from about 10 to about 30 amino acids long, from about 1 to about 50 amino acids long, from about 5 to about 40 amino acids long, from about 12 to about 18 amino acids long, or from about 4 to about 25 amino acids long. In some embodiments, the peptide linker is about any of 1, 2, 3, 4, 5, 6, 7, 8, 9, 10, 11, 12, 13, 14, 15, 16, 17, 18, 19 or 20 amino acids long. In some embodiments, the peptide linker is about any of 21, 22, 23, 24, 25, 26, 27, 28, 29, or 30 amino acids long. In some embodiments, the peptide linker is about any of 31, 32, 33, 34, 35, 36, 37, 38, 39, 40, 41, 42, 43, 44, 45, 46, 47, 48, 49, or 50 amino acids long. Preferably, the in vivo function and/or stability of the long-acting NGF polypeptide described herein is optimized by the addition of a linker peptide to prevent potential undesired domain interactions. In some embodiments, the linker length does not exceed the length necessary to prevent undesired domain interactions and/or to optimize biological function and/or stability. In some embodiments, the peptide linker is at most about 30 amino acids in length, such as at most about 20 amino acids in length, or at most about 15 amino acids in length. In some embodiments, the linker is about 5 to about 30 amino acids in length, or about 5 to about 18 amino acids in length.

A peptide linker can have a naturally occurring sequence or a non-naturally occurring sequence. For example, a sequence derived from the hinge region of a heavy chain only antibody can be used as a linker. See, for example, WO1996/34103. In some embodiments, the peptide linker is a human IgG1, IgG2, IgG3, or IgG4 hinge. In some embodiments, the peptide linker is a mutated human IgG1, IgG2, IgG3, or IgG4 hinge. In some embodiments, the linker is a flexible linker. Exemplary flexible linkers include, but are not limited to, glycine polymers (G)ₙ (SEQ ID NO: 73), glycine-serine polymers (including, for example, (GS)ₙ (SEQ ID NO: 74), (GGS)ₙ (SEQ ID NO: 75), (GGGS)ₙ (SEQ ID NO: 76), (GGS)ₙ (GGGS)ₙ (SEQ ID NO: 77), (GSGGS)ₙ (SEQ ID NO: 78), (GGSGS)ₙ (SEQ ID NO: 79), or (GGGGS)ₙ (SEQ ID NO: 70), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11 173-142 (1992)). Exemplary flexible linkers include, but are not limited to GG (SEQ ID NO: 86), GGSG (SEQ ID NO: 87), GGSGG (SEQ ID NO: 88), GSGSG (SEQ ID NO: 89), GSGGG (SEQ ID NO: 90), GGGSG (SEQ ID NO: 91), GSSSG (SEQ ID NO: 92), GGSGGS (SEQ ID NO: 93), SGGGGS (SEQ ID NO: 94), GGGGS (SEQ ID NO: 95), (GA)ₙ (SEQ ID NO: 96, n is an integer of at least 1), GRAGGGGAGGGG (SEQ ID NO: 97), GRAGGG (SEQ ID NO: 98), GSGGGSGGGGSGGGGS (SEQ ID NO: 80), GGGSGGGGSGGGGS (SEQ ID NO: 81), GGGSGGSGGS (SEQ ID NO: 82), GGSGGSGGSGGSGGG (SEQ ID NO: 83), GGSGGSGGGGSGGGGS (SEQ ID NO: 84), GGSGGSGGSGGSGGSGGS (SEQ ID NO: 85), GGGGSGGGGSGGGGS (SEQ ID NO: 68), GGGGGGSGGGGGGGGSA (SEQ ID NO: 69), GSGGGSGGGGSGGGGSGGGGS (SEQ ID NO: 71), KTGGGSGGGS (SEQ ID NO: 72) and the like. In some embodiments, the linker comprises the sequence of ASTKGP (SEQ ID NO: 99). The ordinarily skilled artisan will recognize that design of a long-acting NGF polypeptide can include linkers that are all or partially flexible, such that the linker can include a flexible linker portion as well as one or more portions that confer less flexible structure to provide a desired long-acting NGF polypeptide structure and function. In some embodiments, the peptide linker is enriched in serine-glycine. In some embodiments, the peptide linker comprises the amino acid sequence of any one of SEQ ID NOs: 68-72. In some embodiments, the peptide linker comprises the amino acid sequence of (GGGGS)ₙ (SEQ ID NO: 70), wherein n is an integer of 1, 2, 3, 4, 5, or 6, preferably n is an integer of 2 to 6, more preferably n is an integer of 3 or 4. In some embodiments, the peptide linker comprises (or consists essentially of, or consists of) the amino acid sequence of SEQ ID NO: 68 or 69.

Other linker considerations include the effect on physical or pharmacokinetic properties of the resulting long-acting NGF polypeptide, such as solubility, lipophilicity, hydrophilicity, hydrophobicity, stability (more or less stable as well as planned degradation), rigidity, flexibility, immunogenicity, NGF moiety/NGF receptor binding, the ability to be incorporated into a micelle or liposome, and the like.

### Binding Affinity

Binding affinity of a molecule (e.g., NGF moiety, or NGF polypeptide comprising an NGF moiety) and its binding partner (e.g., an NGF receptor such as TrkA) can be determined experimentally by any suitable ligand binding assays or antibody/antigen binding assays known in the art, e.g., Western blots, sandwich enzyme-linked immunosorbent assay (ELISA), Meso Scale Discovery (MSD) electrochemiluminescence, bead based multiplex immunoassays (MIA), RIA, Surface Plasma Resonance (SPR), ECL, IRMA, EIA, Biacore assay, Octet analysis, peptide scans, etc. For example, easy analysis is possible by using an NGF moiety, an NGF polypeptide comprising an NGF moiety, or its receptor (e.g., TrkA) or subunits thereof marked with a variety of marker agents, as well as by using BiacoreX (Amersham Biosciences), which is an over-the-counter, measuring kit, or similar kit, according to the user's manual and experiment operation method attached with the kit.

In some embodiments, protein microarray is used for analyzing the interaction, function and activity of the NGF moiety or long-acting NGF polypeptide described herein to its receptor, on a large scale. The protein chip has a support surface bound with a range of capture proteins (e.g., NGF receptor or subunits thereof). Fluorescently labeled probe molecules (e.g., NGF moiety or long-acting NGF polypeptide described herein) are then added to the array and upon interaction with the bound capture protein, a fluorescent signal is released and read by a laser scanner.

Binding affinity can also be measured using SPR (Biacore T-200). For example, anti-human IgG antibody is coupled to the surface of a CM-5 sensor chip using EDC/NHS chemistry. Then human TrkA-Fc fusion protein is used as the captured ligand over this surface. Serial dilutions of NGF moiety or long-acting NGF polypeptide described herein are allowed to bind to the captured ligands, and the binding and dissociation of NGF to TrkA can be monitored in real time. Equilibrium dissociation constant (K_{d}) and dissociation rate constant can be determined by performing kinetic analysis using BIA evaluation software.

In some embodiments, the K_{d} of the binding between the NGF moiety or long-acting NGF polypeptide described herein and its receptor (e.g., TrkA) or subunits thereof is about any of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, the K_{d} of the binding between a wildtype NGF and its receptor (e.g., TrkA) or subunits thereof is similar to (e.g., equal to) or at least about 1.5 times (such as at least about any of 2, 3, 4, 5, 6, 7, 8, 9, 10, 20, 30, 40, 50, 60, 70, 80, 90, 100, 500, or 1000 times) of the K_{d} of the binding between the NGF moiety or long-acting NGF polypeptide described herein and the same receptor (e.g., TrkA) or subunits thereof. In some embodiments, the K_{d} of the binding between an NGF moiety or a long-acting NGF polypeptide described herein and its receptor (e.g., P75) or subunits thereof is similar to (e.g., equal to) or at least about 2 times (such as at least about any of 3, 4, 5, 6, 7, 8, 9, 10, 20, 30, 40, 50, 60, 70, 80, 90, 100, 500, or 1000 times) of the K_{d} of the binding between a wildtype NGF and the same receptor (e.g., P75) or subunits thereof.

In some embodiments, the NGF moiety comprises a mutation or a modification (e.g., post-translational modification), and the K_{d} of the binding between the mutant/modified NGF moiety (or long-acting NGF polypeptide) and its receptor (e.g., TrkA) or subunits thereof is similar to (e.g., equal to) or at least about 2 times (such as at least about any of 3, 4, 5, 6, 7, 8, 9, 10, 20, 30, 40, 50, 60, 70, 80, 90, 100, 500, or 1000 times) of the K_{d} of the binding between a wildtype NGF and the same receptor (e.g., TrkA). In some embodiments, the NGF moiety comprises a mutation or a modification (e.g., post-translational modification), and the K_{d} of the binding between the wildtype NGF and its receptor (e.g., P75) or subunits thereof is similar to (e.g., equal to) or at least about 2 times (such as at least about any of 3, 4, 5, 6, 7, 8, 9, 10, 20, 30, 40, 50, 60, 70, 80, 90, 100, 500, or 1000 times) of the K_{d} of the binding between the mutant/modified NGF moiety (or long-acting NGF polypeptide) and the same receptor (e.g., P75).

### Bioactivity

Various methods for determining the bioactivities of NGF, NGF moieties, or long-acting NGF polypeptides described herein are known in the art. For example, bioactivities can be assessed by TF-1 cell proliferation assay, such as that described in CN103376248A and CN108727486A, the contents of which are incorporated herein by reference in their entirety. Also see Example 4 below. Bioactivities can also be determined based on the ability of promoting superior cervical ganglion (SCG) growth in newborn rats (e.g., see Example 5 below), or promoting dorsal root ganglion growth in chicken embryos (e.g., see WO2017157326, the content of which is incorporated herein by reference in its entirety). Bioactivities can also be determined based on whether there is i) an improvement in wound healing, such as in aminal model/patient of diabetic neuropathy (e.g., see Example 7); ii) an improvement in spatial cognition, memory, and/or learning ability, such as in aminal model/patient of Alzheimer's disease (e.g., see Example 8); iii) an improvement in ovarian granulosa-like tumor cell line proliferation and/or estrogen secretion, and/or a reverse of the reduction of follicles in aminal model/patient of premature ovarian failure (e.g., see Example 9); iv) a rescue of the reduction in sperm number and/or motility, and/or a therapeutic effect on testicular seminiferous tubule atrophy, seminiferous tubule spermatogenesis disorder, and/or epididymal duct cell fragments, in aminal model/patient of spermatogenic disorder (e.g., see Example 10); or v) a restoration of the integrity of damaged cornea corneal (e.g., by fluorescein sodium staining assay), and/or a rescue of the damaged corneal nerve (e.g., by corneal nerve length measurement), in aminal model/patient of neurotrophic keratitis (e.g., see Example 11); etc. Any applicable assays known in the art can be adapted to test bioactivities of NGF moieties or long-acting NGF polypeptides described herein.

A bioassay focuses on biological activity of NGF and using it as a read out. In a bioassay, the activity of a sample is tested on a sensitive cell line (e.g., primary cell cultures or in vitro adapted cell lines that are dependent and/or responsive to the test sample) or an animal model/human of NGF-related disease, and the results of this activity (e.g., cellular proliferation) are compared to a standard NGF preparation or control (e.g., mouse NGF, mNGF118, or known long-acting NGF polypeptide). Other aspects of biological activity of NGF include: (i) supporting neuronal survival; (ii) promoting neurite outgrowth; (iii) enhancing neurochemical differentiation; (iv) promoting the proliferation of pancreatic β cells; (v) inducing innate and/or acquired immunity; (vi) restoring damaged nerve cells and/or preventing damage (e.g., in neurotrophic keratitis); (vii) promoting the proliferation and/or estrogen secretion of ovarian follicle cells; (viii) promoting wound healing (e.g., in diabetic having neurodegenerative disease (e.g., Alzheimer's disease); (x) treating and/or preventing neurodegeneration; (xi) treating testicular seminiferous tubule atrophy, seminiferous tubule spermatogenesis disorder, and/or epididymal duct cell fragments; (xii) rescuing the reduction of sperm number and/or motility, or increasing sperm number and/or motility (e.g., in spermatogenic disorder); and/or (xiii) reversing the reduction of the number and/or function of ovarian follicles, or increasing the number and/or function of ovarian follicles (e.g., in premature ovarian failure). In vitro and/or in vivo assays to measure all of these activities are available, such as neuronal survival assays or neurite outgrowth assays.

For example, in a TF-1 cell proliferation assay, serial dilutions of samples (e.g., long-acting NGF polypeptides) and control (e.g., vehicle or SuTaiSheng® mouse NGF) are prepared in a 96-well plate, then TF-1 cells are added into each well and incubated under 37° C., 5% CO₂ in a humid incubator. A few days (e.g., 3 days) after incubation, add MTS solution into each well of the cell suspension, incubate under 37° C., 5% CO₂ for 3 hours. Then absorbance under 490 nm and 650 nm can be measured in a spectrophotometer, to indicate how NGF moieties or long-acting NGF polypeptides promote TF-1 cell proliferation. Data can be normalized to the control sample. Also see Example 4 for exemplary method.

Cell signaling assays can also be used to test bioactivities of NGF moieties or long-acting NGF polypeptides described herein. Various cell signaling assay kits are commercially available, for example, to detect analytes produced during enzymatic reactions involved in signaling such as ADP, AMP, UDP, GDP, and growth factors, or phosphatase assays, to quantify both total and phosphorylated forms of signaling proteins. For example, after incubating the cells with NGF moieties or long-acting NGF polypeptides described herein, to determine whether a particular kinase is active, the cell lysate is exposed to a known substrate for the enzyme in the presence of radioactive phosphate. The products are separated by electrophoresis (with or without immunoprecipitation), then the gel is exposed to X-ray film to determine whether the proteins incorporated the isotope. In some embodiments, the bioactivities of NGF moieties or long-acting NGF polypeptides described herein on cells are measured by immunohistochemistry to locate signaling proteins. For example, antibodies to the signal proteins themselves or signal proteins in their activated state can be used. These antibodies have recognition epitopes that include the phosphate or other activating conformation. In some embodiments, movement of specific signaling proteins (e.g., nuclear translocation of signaling molecules) can be tracked by incorporating a fluorescent protein gene, e.g., green fluorescent protein (GFP), into genetic vectors encoding the protein to be studied. In some embodiments, bioactivities of NGF moieties or long-acting NGF polypeptides described herein on cells are tested by western blots. For example, all tyrosine-phosphorylated proteins (or other phosphorylated amino acids, e.g., serine or threonine) can be detected with an anti-phosphotyrosine antibody (or antibodies against other phosphorylated amino acids) on a Western blot of cell lysates obtained after stimulation in a temporal sequence. In some embodiments, the bioactivities of NGF moieties or long-acting NGF polypeptides described herein on cells can be measured by immunoprecipitation. For example, primary antibodies to a specific signaling protein or all tyrosine-phosphorylated proteins are cross-linked to the beads. The cells after incubating with NGF moieties or long-acting NGF polypeptides described herein are lysed in buffer containing protease inhibitors and then incubated with the antibody-coated beads. The proteins are separated by using SDS electrophoresis, and then the proteins are identified by using the procedures described for Western blots. In some embodiments, glutathione S-transferase (GST) binding, or “pull-down” assay, can also be used, which determines direct protein-protein (e.g., signaling protein) interactions.

For example, RAS/ERK1/2 signaling can be measured to reflect NGF bioactivity in promoting cell growth, for example, by phosphorylation of ERK1/2 using any suitable method known in the art. For example, ERK1/2 phosphorylation can be measured using antibodies specific for the phosphorylated version of the molecule (optionally in combination with flow cytometry analysis). For example, chicken embryonic dorsal root ganglias (DRGs) or TF-1 cells are incubated at 37° C. with NGF moieties or long-acting NGF polypeptides described herein. After incubation, cells are immediately fixed to preserve the phosphorylation status and permeabilized. The cells are stained with antibodies against phosphorylated ERK1/2, e.g., Alexa488-conjugated anti-ERK1/2 pT202/pY204 (BD Biosciences), and analyzed by flow cytometry. PI 3-kinase signaling can be measured using any suitable method known in the art to reflect NGF bioactivity, too. For example, PI 3-kinase signaling can be measured using antibodies that are specific for phospho-S6 ribosomal protein (optionally in conjunction with flow cytometry analysis).

Bioactivities of NGF moieties or long-acting NGF polypeptides described herein can also be reflected by in vivo or ex vivo experiments, for example, by measuring the proliferation of indicator cells, by measuring the induction or inhibition of signaling transduction, by measuring tissue volume and/or weight, etc.

For example, in an SCG in vivo growth assay, samples (e.g., long-acting NGF polypeptides) and control (e.g., PBS or SuTaiSheng® mouse NGF) can be subcutaneously administered to the neck of newborn rats, either with single injection or multiple injections. These rats are sacrificed for SCG dissection after a few days post-injection. SCG can be weighed and recorded for morphology to study the bioactivity of NGF moieties or long-acting NGF polypeptides in promoting SCG in vivo growth. Also see Example 5 for exemplary method.

In a dorsal root ganglion growth assay, chicken embryo dorsal root ganglions (e.g., 8-day old) can be added into medium containing various concentrations of either samples (e.g., long-acting NGF polypeptides) or control (e.g., PBS or SuTaiSheng® mouse NGF), and culture in a saturated humidity incubator in a 5% CO₂ and at 37° C., e.g., for 24 hours. The growth condition of dorsal root ganglions is monitored, which can reflect the bioactivity of NGF moieties or long-acting NGF polypeptides in promoting dorsal root ganglion growth. Specific bioactivity of the samples can also be calculated if an NGF standard is involved in the assay, reflected in AU/mg. Specific activity of the sample to be tested (AU/mg)=activity of the reference product (AU/ml)×[pre-dilution factor of the sample×activity of the corresponding reference product at the dilution point (AU/ml)/actual activity of the reference product (AU/ml)]. Also see Example 5 of WO2017157326 for exemplary method.

In some embodiments, the NGF moiety (or long-acting NGF polypeptide) described herein comprises a mutation or a modification (e.g., post-translational modification) that retains/enhances/reduces its bioactivity compared to the wildtype NGF (or polyptide comprising wildtype NGF). In some embodiments, the mutated or modified NGF moiety or long-acting NGF polypeptide described herein has similar (e.g., equal) or at least about 2 times (such as at least about any of 3, 4, 5, 6, 7, 8, 9, 10, 20, 30, 40, 50, 60, 70, 80, 90, 100, 500, 1000, 5000, or 10000 times or more) of the bioactivity (e.g., promoting cell growth) compared to that of wildtype NGF (or polypeptide comprising wildtype NGF). In some embodiments, the long-acting NGF polypeptide described herein has similar (e.g., equal) or at least about 1.1 times (such as at least about any of 1.2, 1.3, 1.4, 1.5, 1.6, 1.7, 1.8, 1.9, 2, 2.5, 3, 4, 5, 6, 7, 8, 9, 10, 20, 30, 40, 50, 60, 70, 80, 90, 100 times, or more) of the bioactivity (e.g., promoting cell growth, wound healing) compared to an NGF moiety (e.g., a corresponding NGF moiety of the long-acting NGF polypeptide).

### Pharmacokinetics (PK)

Pharmacokinetics (PK) refers to the absorption, distribution, metabolism, and excretion of a drug (e.g., NGF moiety or long-acting NGF polypeptide described herein) once it has been administered to a subject. Pharmacokinetic parameters that may be useful in determining clinical utility include but are not limited to serum/plasma concentration, serum/plasma concentration over time, maximum serum/plasma concentration (Cₘₐₓ), time to reach maximum concentration (Tₘₐₓ), half-life (t_{1/2}), area under concentration time curve within the dosing interval (AUC_{τ}), etc.

Techniques for obtaining a PK curve of a drug, such as NGF moiety or long-acting NGF polypeptide described herein, or a reference drug (e.g., SuTaiSheng® mouse NGF), are known in the art. See, e.g., Heller et al., Annu Rev Anal Chem, 11, 2018; and Ghandforoush-Sattari et al., J Amino Acids, Article ID 346237, Volume 2010. In some embodiments, the PK curves of the NGF moiety or long-acting NGF polypeptide described herein in the individual is measured in a blood, plasma, or serum sample from the individual. In some embodiments, the PK curves of the NGF moiety or long-acting NGF polypeptide described herein in the individual is measured using a mass spectrometry technique, such as LC-MS/MS, or ELISA. PK analysis on PK curves can be conducted by any methods known in the art, such as non-compartmental analysis, e.g., using PKSolver V2 software (Zhang Y. et al., “PKSolver: An add-in program for pharmacokinetic and pharmacodynamic data analysis in Microsoft Excel,” Comput Methods Programs Biomed. 2010; 99 (3): 306-1). Also see Example 6 for exemplary method.

“C” denotes the concentration of drug (e.g., NGF moiety or long-acting NGF polypeptide) in blood plasma, serum, or in any appropriate body fluid or tissue of a subject, and is generally expressed as mass per unit volume, for example nanograms per milliliter. For convenience, the concentration of drug in serum or plasma is referred to herein as “serum concentration” or “plasma concentration.” The serum/plasma concentration at any time following drug administration (e.g., NGF moiety or long-acting NGF polypeptide, such as i.v., i.p., or s.c. administration) is referenced as Cₜᵢₘₑ or Cₜ. The maximum serum/plasma drug concentration during the dosing period is referenced as Cₘₐₓ, while Cₘᵢₙ refers to the minimum serum/plasma drug concentration at the end of a dosing interval; and Cₐᵥₑ refers to an average concentration during the dosing interval.

The term “bioavailability” refers to an extent to which—and sometimes rate at which—the drug (e.g., NGF moiety or long-acting NGF polypeptide) enters systemic circulation, thereby gaining access to the site of action.

“AUC” is the area under the serum/plasma concentration-time curve and is considered to be the most reliable measure of bioavailability, such as area under concentration time curve within the dosing interval (AUC_{τ}), “overall exposure” or “total drug exposure across time” (AUC₀₋ₗₐₛₜ or AUC_{0-inf}), area under concentration time curve at time t post-administration (AUC₀₋ₜ), etc.

Serum/plasma concentration peak time (Tₘₐₓ) is the time when peak serum/plasma concentration (Cₘₐₓ) is reached after administration of a drug (e.g., NGF moiety or long-acting NGF polypeptide).

Half-life (t_{1/2}) is the amount of time required for the drug concentration (e.g., NGF moiety or long-acting NGF polypeptide) measured in plasma or serum (or other biological matrices) to be reduced to exactly half of its concentration or amount at certain time point. For example, after IV dosing, the drug concentrations in plasma or serum decline due to both distribution and elimination. In a plasma or serum profile of drug concentration over time post-IV doing, the first phase or rapid decline is considered to be primarily due to distribution, while the later phase of decline is usually slower and considered to be primarily due to elimination, although both processes occur in both phases. Distribution is assumed to be complete after sufficient time. In general, the elimination half-life is determined from the terminal or elimination (dominant) phase of the plasma/serum concentration versus time curve. See, e.g., Michael Schrag and Kelly Regal, “Chapter 3—Pharmacokinetics and Toxicokinetics” of “A Comprehensive Guide to Toxicology in Preclinical Drug Development”, 2013.

In some embodiments, the long-acting NGF polypeptide described herein has a half-life (e.g., i.v., s.c., or intramuscular administration, such as to human) of at least about 5 hours, such as at least about any of 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 150, 200, 250, or 300 hours, or longer. In some embodiments, the long-acting NGF polypeptide described herein has a half-life (e.g., i.v. administration, such as to human) of about 5 hours to about 300 hours, such as any of about 8 hours to about 100 hours, about 10 hours to about 60 hours, about 15 hours to about 60 hours, or about 20 hours to about 58 hours. In some embodiments, the long-acting NGF polypeptide described herein is administered in a single administration, such as a single intravenous injection or infusion, a single intramuscular injection, or a single subcutaneous injection. In some embodiments, the circulating half-life of the long-acting NGF polypeptide described herein is about 55 hours.

In some embodiments, the half-life of the NGF moiety is about 1 hour to about 2.5 hours, such as about 1.5 hour to about 2.4 hours. In some embodiments, the circulating half-life of the long-acting NGF polypeptide described herein is at least about 5 folds of that of the corresponding NGF moiety (i.e., the NGF moiety contained within the long-acting NGF polypeptide but without Fc fusion) or a wildtype NGF, such as at least about any of 6, 7, 8, 9, 10, 11, 12, 13, 14, 15, 16, 17, 18, 19, 20, 25, 30, 35, 40, 50, 60, 70, 80, 90, or 100 folds or more of that of the corresponding NGF moiety or a wildtype NGF.

### Pain-Causing Activity

NGF is a well-validated target for pain as it causes pain in animals and humans. In adults, NGF, in particular, promotes the health and survival of a subset of central and peripheral neurons (Huang & Reichardt, Ann. Rev. Neurosci. 24:677-736 (2001)). NGF also contributes to the modulation of the functional characteristics of these neurons and exerts tonic control over the sensitivity, or excitability, of sensory pain receptors called nociceptors (Priestley et al., Can. J. Physiol. Pharmacol. 80:495-505 (2002); Bennett, Neuroscientist 7:13-17 (2001)). Nociceptors sense and transmit to the central nervous system the various noxious stimuli that give rise to perceptions of pain (nociception). NGF receptors are located on nociceptors. The expression of NGF is increased in injured and inflamed tissue and is upregulated in human pain states. NGF-induced nociception/pain is mediated by the high affinity NGF receptor, trkA (tyrosine receptor kinase A) (Sah, et al., Nat. Rev. Drug Disc. 2:460-72 (2003)).

In its broadest usage, “pain” refers to an experiential phenomenon that is highly subjective to the individual experiencing it, and is influenced by the individual's mental state, including environment and cultural background. “Physical” pain can usually be linked to a stimulus perceivable to a third party that is causative of actual or potential tissue damage. In this sense, pain can be regarded as a “sensory and emotional experience associated with actual or potential tissue damage, or described in terms of such damage,” according to the International Association for the Study of Pain (IASP). However, some instances of pain have no perceivable cause. For example, psychogenic pain, including exacerbation of a pre-existing physical pain by psychogenic factors or syndromes of a sometimes persistent, perceived pain in persons with psychological disorders without any evidence of a perceivable cause of pain.

Pain includes nociceptive pain, neuropathic/neurogenic pain, breakthrough pain, allodynia, hyperalgesia, hyperesthesia, dysesthesia, paresthesia, hyperpathia, phantom limb pain, psychogenic pain, anesthesia dolorosa, neuralgia, neuritis. Other categorizations include malignant pain, anginal pain, and/or idiopathic pain, complex regional pain syndrome I, complex regional pain syndrome II. Types and symptoms of pain need not be mutually exclusive. These terms are intended as defined by the IASP.

Nociceptive pain is initiated by specialized sensory nociceptors in the peripheral nerves in response to noxious stimuli, encoding noxious stimuli into action potentials. Nociceptors, generally on A8 fibers and (Polymodal) C fibers, are free nerve endings that terminate just below the skin, in tendons, joints, and in body organs. The dorsal root ganglion (DRG) neurons provide a site of communication between the periphery and the spinal cord. The signal is processed through the spinal cord to the brainstem and thalamic sites and finally to the cerebral cortex, where it usually (but not always) elicits a sensation of pain. Nociceptive pain can result from a wide variety of a chemical, thermal, biological (e.g., inflammatory) or mechanical events that have the potential to irritate or damage body tissue, which are generally above a certain minimal threshold of intensity required to cause nociceptive activity in nociceptors.

Neuropathic pain is generally the result of abnormal functioning in the peripheral or central nervous system, giving rise to peripheral or central neuropathic pain, respectively. Neuropathic pain is defined by the IASP as pain initiated or caused by a primary lesion or dysfunction in the nervous system. Neuropathic pain often involves actual damage to the nervous system, especially in chronic cases. Inflammatory nociceptive pain is generally a result of tissue damage and the resulting inflammatory process. Neuropathic pain can persist well after (e.g., months or years) beyond the apparent healing of any observable damage to tissues.

In cases of neuropathic pain, sensory processing from an affected region can become abnormal and innocuous stimuli (e.g., thermal, touch/pressure) that would normally not cause pain may do so (i.e., allodynia) or noxious stimuli may elicit exaggerated perceptions of pain (i.e., hyperalgesia) in response to a normally painful stimulus. In addition, sensations similar to electric tingling or shocks or “pins and needles” (i.e., paresthesias) and/or sensations having unpleasant qualities (i.e., dysesthesias) may be elicited by normal stimuli. Breakthrough pain is an aggravation of pre-existing chronic pain. Hyperpathia is a painful syndrome resulting from an abnormally painful reaction to a stimulus. The stimulus in most of the cases is repetitive with an increased pain threshold, which can be regarded as the least experience of pain that a patient can recognize as pain.

Examples of neuropathic pain include tactile allodynia (e.g., induced after nerve injury) neuralgia (e.g., post herpetic (or post-shingles) neuralgia, trigeminal neuralgia), reflex sympathetic dystrophy/causalgia (nerve trauma), components of cancer pain (e.g., pain due to the cancer itself or associated conditions such as inflammation, or due to treatment such as chemotherapy, surgery or radiotherapy), phantom limb pain, entrapment neuropathy (e.g., carpal tunnel syndrome), and neuropathies such as peripheral neuropathy (e.g., due to diabetes, HIV, chronic alcohol use, exposure to other toxins (including many chemotherapies), vitamin deficiencies, and a large variety of other medical conditions). Neuropathic pain includes pain induced by expression of pathological operation of the nervous system following nerve injury due to various causes, for example, surgical operation, wound, shingles, diabetic neuropathy, amputation of legs or arms, cancer, and the like. Medical conditions associated with neuropathic pain include traumatic nerve injury, stroke, multiple sclerosis, syringomyelia, spinal cord injury, and cancer.

A pain-causing stimulus often evokes an inflammatory response which itself can contribute to an experience of pain. In some conditions pain appears to be caused by a complex mixture of nociceptive and neuropathic factors. For example, chronic pain often comprises inflammatory nociceptive pain or neuropathic pain, or a mixture of both. An initial nervous system dysfunction or injury may trigger the neural release of inflammatory mediators and subsequent neuropathic inflammation. For example, migraine headaches can represent a mixture of neuropathic and nociceptive pain. Also, myofascial pain is probably secondary to nociceptive input from the muscles, but the abnormal muscle activity may be the result of neuropathic conditions.

In some embodiments, the long-acting NGF polypeptide described herein has reduced or no pain-causing activity in the subject, such as compared to the corresponding NGF moiety without Fc fusion, compared to a wildtype NGF, or compared to other NGF-Fc fusion proteins not described herein (hereinafter referred to as “reference NGF construct”). In some embodiments, the pain is acute pain, short-term pain, persistent or chronic nociceptive pain, or persistent or chronic neuropathic pain. In some embodiments, the long-acting NGF polypeptide described herein causes at least about 10% less pain compared to a reference NGF construct (e.g., wildtype β-NGF), such as at least about any of 20%, 25%, 30%, 35%, 40%, 45%, 50%, 55%, 60%, 70%, 80%, 90%, 95%, or 100% less pain compared to a reference NGF construct. In some embodiments, the long-acting NGF polypeptide described herein does not cause pain when administered to a subject.

Pain-causing activity can be measured by any methods known in the art, such as those described in WO2017157325, WO2017157326, and CN108727486A, the contents of which are incorporated herein by reference in their entirety. In some embodiments, pain is measured by pain threshold, the higher the pain threshold, the less the pain.

For example, pain-causing activity can be measured by asking a patient to rate the quality and intensity of pain experienced according to a number of different scales. A verbal pain scale uses words to describe a range from no pain, mild pain, moderate pain and severe pain with a score from 0-3 assigned to each. Alternatively, a patient may be asked to rate their pain according to a numerical pain scale from 0 (no pain) to 10 (worst possible pain). On a visual analog scale (VAS) a vertical or horizontal line has words to describe pain from no pain to worst possible pain and the patient is asked to mark the line at the point that represents their current level of pain. The McGill pain index enables patients to describe both the quality and intensity of pain by selecting words that best describe their pain from a series of short lists e.g. pounding, burning, pinching. Other pain scales can be used for adults who experience difficulty using VAS or numerical scales e.g. FACES or for non-verbal patients, e.g. behavioural rating scale. The functional activity score relates how impeded a patient is by their pain by asking them to carry out a task related to the painful area. Improvements in pain score using these types of scale, e.g., compared to reference NGF constructs, would potentially indicate reduced side effect of causing pain for the test NGF constructs (e.g., long-acting NGF polypeptide described herein).

In some embodiments, pain-causing activity of the long-acting NGF polypeptides described herein can be tested on mice by hot plate method (54-55° C.) to determine pain threshold. Briefly, response eligible mice are anesthetized, then a nerve clamping method is used to create a mouse sciatic nerve injury model, while the sciatic nerve is only separated but not clamped in the sham operation group. Then mice are divided into three groups: a sham operation group, an injury control group (normal saline), and an experimental group (treated with long-acting NGF polypeptides described herein, and/or control NGF such as a mouse β-NGF). The pain threshold of each mouse is indicated by the latency of licking hind feet, which can be measured before the surgery and at different timepoints post-surgery. Increase in pain threshold %=(Pain threshold on day 10 after injury−Pain threshold before injury)×100%/Pain threshold before injury.

Pain-causing activity of the long-acting NGF polypeptides described herein can also be tested on mice by measuring curved claw response in mouse under mechanical stimulation, to determine pain threshold. It can be tested under short-term pain causing condition or long-term pain causing condition. Briefly, response eligible mice are s.c. injected to feet with either vehicle or long-acting NGF polypeptides described herein (or control NGF such as a mouse β-NGF; can be at various concentrations), then measured for curved claw response under mechanical stimulation post-injection (e.g., at different timepoints), which reflects pain threshold after treatment.

Pain-causing activity of the long-acting NGF polypeptides described herein can also be tested by behavior tests. For example, vehicle or long-acting NGF polypeptides described herein (or control NGF such as a mouse β-NGF; can be at various concentrations) can be administered to the joints of mice, then whether the samples cause pain can be examined by recording leg lifting maintenance time and leg lifting numbers post-administration (e.g., at different timepoints), to calculate overall leg lifting duration. Shorter overall leg lifting duration indicates less pain-causing activity.

### Stability

In some embodiments, the long-acting NGF polypeptides described herein have excellent stability, such as physical stability, chemical stability, and/or biological stability. In some embodiments, the long-acting NGF polypeptides described herein have excellent thermal stability, such as high melting temperature (Tm) and/or high onset aggregation temperature (Tagg). In some embodiments, the long-acting NGF polypeptides described herein have superior stability under accelerated stress (e.g., high temperature), such as less or no fragmentation, aggregate formation, and/or aggregate increment.

Stability of protein, in particular the susceptibility to aggregation, is primarily determined by the conformational and the colloidal stability of the protein molecules. It is generally believed that the first step in non-native protein aggregation, which is the most prevalent form of aggregation, is a slight perturbation of the molecular structure, e.g., a partial unfolding of the protein, i.e., a conformational change. This is determined by the conformational stability of the protein. In the second step, the partially unfolded molecules then come into close proximity, being driven by diffusion and random Brownian motion, to form aggregates. This second step is primarily governed by the colloidal stability of the molecules (see Chi et al., Roles of conformational stability and colloidal stability in the aggregation of recombinant human granulocyte colony stimulating factor. Protein Science, 2003 May; 12 (5): 903-913). As used herein, the term “stability” generally is related to maintaining the integrity or to minimizing the degradation, denaturation, aggregation or unfolding of a biologically active agent such as a protein. As used herein, “improved stability” generally means that, under conditions known to result in degradation, denaturation, aggregation or unfolding, the protein (e.g., long-acting NGF polypeptides described herein) of interest maintains greater stability compared to a control protein (e.g., other NGF-Fc fusion proteins).

Differential scanning calorimetry (DSC) and differential scanning fluorimetry (DSF) are well known techniques in the art that are used to predict the stability of a protein formulation. Specifically, these techniques can be used to determine the unfolding temperature (Tm) of a protein in given formulation. It is standard in the art to correlate high Tm measurements for a protein in given formulation with more robust and stable protein formulations for long-term, shelf-stable storage.

A “stable” protein (or formulation), e.g., a long-acting NGF polypeptide described herein, essentially retains its physical stability and/or chemical stability and/or biological activity during the manufacturing process and/or upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. (1993) Adv. Drug Delivery Rev. 10:29-90. For example, in one embodiment, the stability of the protein is determined according to the percentage of monomer protein in the solution, with a low percentage of degraded (e.g., fragmented) and/or aggregated protein. Preferably, the protein (or formulation) is stable at room temperature (about 30° C.) or at 40° C. for at least 1 month and/or stable at about 2-8° C. for at least 6 months, or for at least 1 year or for at least 2 years. Furthermore, the protein (or formulation) is preferably stable following freezing (to, e.g., −70° C.) and thawing, hereinafter referred to as a “freeze/thaw cycle.”

A protein, e.g., a long-acting NGF polypeptide described herein, “retains its physical stability” in a formulation if it shows substantially no signs of instability, e.g., aggregation, precipitation and/or denaturation, upon visual examination of color and/or clarity or as measured by UV light scattering or by size exclusion chromatography. Aggregation is a process whereby individual protein molecules or complexes associate covalently or non-covalently to form aggregates. Aggregation can proceed to the extent that a visible precipitate is formed.

A protein, e.g., a long-acting NGF polypeptide described herein, “retains its chemical stability” in a formulation, if the chemical stability at a given time is such that the protein is considered to still retain its biological activity (e.g., as mentioned in “Bioactivity” subsection above). Chemical stability can be assessed by, e.g., detecting and quantifying chemically altered forms of the protein. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation or oxidation) which can be evaluated by ion-exchange chromatography, for example.

A protein, e.g., a long-acting NGF polypeptide described herein, “retains its biological activity” in a formulation, if the protein, in a pharmaceutical formulation is biologically active for its intended purpose. For example, biological activity is retained if the biological activity of the protein, in the formulation is within about 30%, about 20%, or about 10% (within the errors of the assay) of the biological activity exhibited at the time the formulation was prepared.

One of skill in the art will appreciate that stability of a protein (e.g., long-acting NGF polypeptides described herein) is dependent on other features in addition to the composition of the formulation. For example, stability can be affected by temperature, pressure, humidity, pH, and external forms of radiation. Stability of a protein (e.g., long-acting NGF polypeptides described herein) in a protein formulation can be determined by various means. In some embodiments, the protein stability is determined by size exclusion chromatography (SEC). SEC separates analytes (e.g., macromolecules such as proteins) on the basis of a combination of their hydrodynamic size, diffusion coefficient, and surface properties. Thus, for example, SEC can separate long-acting NGF polypeptides described herein in their natural three-dimensional conformation from proteins in various states of denaturation, and/or proteins that have been degraded. In SEC, the stationary phase is generally composed of inert particles packed into a dense three-dimensional matrix within a glass or steel column. The mobile phase can be pure water, an aqueous buffer, an organic solvent, mixtures of these, or other solvents. The stationary-phase particles have small pores and/or channels which will only allow species below a certain size to enter. Large particles are therefore excluded from these pores and channels, but the smaller particles are removed from the flowing mobile phase. The time particles spend immobilized in the stationary-phase pores depends, in part, on how far into the pores they can penetrate. Their removal from the mobile phase flow causes them to take longer to elute from the column and results in a separation between the particles based on differences in their size. See Example 3 providing exemplary methods.

In some embodiments, SEC is combined with an identification technique to identify or characterize proteins (e.g., long-acting NGF polypeptides described herein), or fragments thereof. Protein identification and characterization can be accomplished by various techniques, including but not limited chromatographic techniques, e.g., high-performance liquid chromatography (HPLC), Capillary Electrophoresis-Sodium Dodecyl Sulfate (CE-SDS), immunoassays, electrophoresis, ultra-violet/visible/infrared spectroscopy, raman spectroscopy, surface enhanced raman spectroscopy, mass spectroscopy, gas chromatography, static light scattering (SLS), Fourier Transform Infrared Spectroscopy (FTIR), circular dichroism (CD), urea-induced protein unfolding techniques, intrinsic tryptophan fluorescence, differential scanning calorimetry, and/or ANS protein binding.

In some embodiments, sample formulations (e.g., comprising the long-acting NGF polypeptides described herein) and reference formulations (e.g., comprising other NGF-Fc fusion proteins, or standards) are optionally assayed prior to a treatment phase to determine the content of monomer, aggregated and/or fragmented protein (and/or fragmentation increase %, aggregation increase %, etc.), such as described below in the Example 3. Subsequently, each of the protein formulations undergoes a treatment phase. For example, each protein formulation may be stored for an extended period (e.g., 3 months, 6 months, 12 months, or longer) at a specific temperature (e.g., 40° C., 25° C., or 5° C.). In some embodiments, the protein formulations undergo a physical stress test such as stir-stress assay. In some embodiments, the protein formulations undergo accelerated stability test, such as treated under accelerated stress, including high temperature (e.g., 40° C.), high humidity, and/or low pH, etc. In some embodiments, the protein formulations undergo cycles of freezing and thawing. In some embodiments, samples of the same protein formulation receive differential treatment, e.g., storage for a period of time in different temperatures. Following the treatment phase, the protein formulations are assayed to determine the content of protein monomer, aggregates and/or fragments (and/or fragmentation increase %, aggregation increase %, etc.). In some embodiments, the protein formulations are treated under continuous heating to measure melting temperature (Tm) and/or onset aggregation temperature (Tagg), such as increasing temperature from about 20° C. to about 95° C. (e.g., with a heating rate of about 0.3° C./min). Tm and Tagg can be measured by changes of fluorescence absorbance and light scattering under 266 nm/473 nm, respectively, on a fluorescence protein analyzer. Higher Tm indicates higher thermal stability. Higher Tagg indicates less prone to aggregation. In some embodiments, the long-acting NGF polypeptides described herein has a Tm of at least about 50° C., such as at least about any of 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., or 75° C. In some embodiments, the long-acting NGF polypeptides described herein has a Tagg of at least about 50° C., such as at least about any of 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., or 85° C.

Stability, such as physical stability of a composition or formulation, may be assessed by methods well-known in the art, including measurement of a sample's apparent attenuation of light (absorbance, or optical density). Such a measurement of light attenuation relates to the turbidity of a formulation. The turbidity of a formulation is partially an intrinsic property of the protein dissolved in solution and is commonly determined by nephelometry, and measured in Nephelometric Turbidity Units (NTU).

The degree of turbidity, e.g., as a function of the concentration of one or more of the components in the solution, e.g., protein and/or salt concentration, is also referred to as the “opalescence” or “opalescent appearance” of a formulation. The degree of turbidity can be calculated by reference to a standard curve generated using suspensions of known turbidity. Reference standards for determining the degree of turbidity for pharmaceutical compositions can be based on the European Pharmacopeia criteria (European Pharmacopoeia, Fourth Ed., Directorate for the Quality of Medicine of the Council of Europe (EDQM), Strasbourg, France). According to the European Pharmacopeia criteria, a clear solution is defined as one with a turbidity less than or equal to a reference suspension which has a turbidity of approximately 3 according to European Pharmacopeia standards. Nephelometric turbidity measurements can detect Rayleigh scatter, which typically changes linearly with concentration, in the absence of association or nonideality effects. Other methods for assessing physical stability of a pharmaceutical protein are well-known in the art, e.g., size-exclusion chromatography or analytical ultracentrifucation.

In some embodiments, stability refers to formulation containing a long-acting NGF polypeptide described herein having low to undetectable levels of particle formation. The phrase “low to undetectable levels of particle formation” as used herein refers to samples containing less than 30 particles/mL, less than 20 particles/ml, less than 20 particles/ml, less than 15 particles/ml, less than 10 particles/ml, less than 5 particles/ml, less than 2 particles/ml or less than 1 particle/ml as determined by HIAC analysis or visual analysis. In some embodiments, no particle in the long-acting NGF polypeptide formulation is detected, either by HIAC analysis or visual analysis.

“Substantial protein aggregation” refers to a level of protein aggregation in a protein formulation that is substantially greater than the level of protein aggregation in a reference protein formulation. The reference protein formulation may be the same protein formulation before a period of storage or before a treatment (e.g., before subjected to a destabilizing condition, such as elevated temperature, humidity, pH, and/or to long term storage.). The reference protein formulation may be a different protein formulation (e.g., other NGF-Fc fusion proteins, or NGF moiety without Fc fusion) tested under the same condition.

“Substantially free of protein aggregation” refers to proteins (or formulations) of the invention that do not have a significantly greater level or percentage of aggregated protein than a reference formulation. For example, this phrase refers to proteins (or formulations) in which the level of protein aggregation is less than about 15%, 14%, 13%, 12%, 11%, 10%, 9%, 8%, 7%, 6%, 5%, 4%, 3%, 2%, 1%, 0.5%, 0.2% or 0.1%. The level of protein aggregation may be determined using standard techniques known in the art, such as described in the Example 3 herein. In some embodiments, the long-acting NGF polypeptide described herein is substantially free of protein aggregation (e.g., under accelerated stability test). In some embodiments, the long-acting NGF polypeptide has at most about 15% protein aggregation, such as at most about any of 14%, 13%, 12%, 11%, 10%, 9%, 8%, 7%, 6%, 5%, 4%, 3%, 2%, 1%, or 0.5% protein aggregation (e.g., under accelerated stability test, such as heating). In some embodiments, the long-acting NGF polypeptide described herein has no protein aggregation (e.g., under accelerated stability test, such as heating). In some embodiments, the long-acting NGF polypeptide has no more than about 15% of aggregation increase, such as no more than any of about 14%, 13%, 12%, 11%, 10%, 9%, 8%, 7%, 6%, 5%, 4%, 3% 2%, or 1% aggregation increase (e.g., under accelerated stability test, such as accelerated heating). In some embodiments, the stability is measured by SEC. In some embodiments, the stability is measured by CE-SDS.

In some embodiments, stability refers to reduced fragmentation of the long-acting NGF polypeptide described herein. The term “low to undetectable levels of fragmentation” as used herein refers to samples containing equal to or more than 80%, 85%, 90%, 95%, 98% or 99% of the total protein, for example, in a single peak as determined by HPSEC, or in multiple peaks (e.g., as many peaks as there are subunits) by reduced Capillary Gel Electrophoresis (rCGE), representing the non-degraded protein or a non-degraded fragment thereof, and containing no other single peaks having more than 5%, more than 4%, more than 3%, more than 2%, more than 1%, or more than 0.5% of the total protein in each. The term “reduced Capillary Gel Electrophoresis” as used herein refers to capillary gel electrophoresis under reducing conditions sufficient to reduce disulfide bonds in an Fc-containing protein, such as the long-acting NGF polypeptide described herein. In some embodiments, the long-acting NGF polypeptide has about 0% to about 15% fragmentation, such as about 0% to about 12% fragmentation (e.g., under accelerated stability test, such as accelerated heating). In some embodiments, the long-acting NGF polypeptide has no more than about 30% of fragmentation, such as no more than any of about 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% fragmentation (e.g., under accelerated stability test, such as accelerated heating). In some embodiments, the long-acting NGF polypeptide has no fragmentation (e.g., under accelerated stability test, such as accelerated heating). In some embodiments, the long-acting NGF polypeptide has no more than about 30% of fragmentation increase, such as no more than any of about 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% fragmentation increase (e.g., under accelerated stability test, such as accelerated heating). In some embodiments, the long-acting NGF polypeptide has at least about 75% main peak, such as at least about any of 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% main peak (e.g., under accelerated stability test, such as accelerated heating). In some embodiments, the stability is measured by SEC. In some embodiments, the stability is measured by CE-SDS.

### Derivatives of Long-Acting NGF Polypeptides

In some embodiments, long-acting NGF polypeptides provided herein may be further modified to comprise additional nonproteinaceous moieties that are known in the art and readily available. The moieties suitable for derivatization of the long-acting NGF polypeptide include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly (n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the long-acting NGF polypeptide may vary, and if more than one polymer is attached, they can be the same or different molecules. In general, the number and/or type of polymers used for derivatization can be determined based on considerations including, but not limited to, the particular properties or functions of the long-acting NGF polypeptide to be improved, whether the long-acting NGF polypeptide derivative will be used in a therapy under defined conditions, etc.

In some embodiments, the long-acting NGF polypeptide described herein further comprises a label selected from the group consisting of a chromophore, a fluorophore (e.g., coumarin, a xanthene, a cyanine, a pyrene, a borapolyazaindacene, an oxazine, and derivatives thereof), a fluorescent protein (e.g., GFP, phycobiliproteins, and derivatives thereof), a phosphorescent dye (e.g., dioxetanes, xanthene, or carbocyanine dyes, lanthanide chelates), a tandem dye (e.g., cyanine-phycobiliprotein derivative and xanthene-phycobiliprotein derivative), a particle (e.g., gold clusters, colloidal gold, microspheres, quantum dots), a hapten, an enzyme (e.g., peroxidase, a phosphatase, a glycosidase, a luciferase), and a radioisotope (e.g., ¹²⁵I, ³H, ¹⁴C, ³²P).

In some embodiments, a long-acting NGF polypeptide may be further modified to comprise one or more biologically active protein, polypeptides or fragments thereof. “Bioactive” or “biologically active”, as used herein interchangeably, means showing biological activity in the body to carry out a specific function. For example, it may mean the combination with a particular biomolecule such as protein, DNA, etc., and then promotion or inhibition of the activity of such biomolecule. In some embodiments, the bioactive protein or fragments thereof include proteins and polypeptides that are administered to patients as the active drug substance for prevention of or treatment of a disease or condition, as well as proteins and polypeptides that are used for diagnostic purposes, such as enzymes used in diagnostic tests or in vitro assays, as well as proteins and polypeptides that are administered to a patient to prevent a disease such as a vaccine. In some embodiments, the bioactive protein or fragments thereof have immune-stimulatory/immune-regulatory, membrane transport, or enzymatic activities. In some embodiments, the biologically active protein, polypeptides or fragments thereof is an enzyme, a hormone, a growth factor, a cytokine, or a mixture thereof. In some embodiments, the biologically active protein, polypeptides or fragments can specifically recognize a target peptide (such as antigen, or other proteins).

In some embodiments, the bioactive protein or fragments thereof that can be comprised within the long-acting NGF polypeptide described herein is an antigen-binding protein (e.g., antibody). In some embodiments, the bioactive protein or fragments thereof that can be comprised within the long-acting NGF polypeptide described herein is an antibody mimetics, which are small engineered proteins comprising antigen-binding domains reminiscent of antibodies (Geering and Fussenegger, Trends Biotechnol., 33 (2): 65-79, 2015). These molecules are derived from existing human scaffold proteins and comprise a single polypeptide. Exemplary antibody mimetics that can be comprised within the long-acting NGF polypeptide described herein can be, but are not limited to, a Designed ankyrin repeat protein (DARPin; comprising 3-5 fully synthetic ankyrin repeats flanked by N- and C-terminal Cap domains), an avidity multimer (avimer; a high-affinity protein comprising multiple A domains, each domain with low affinity for a target), or an Anticalin (based on the scaffold of lipocalins, with four accessible loops, the sequence of each can be randomized). In some embodiments, the bioactive protein or fragments thereof that can be comprised within the long-acting NGF polypeptide described herein is an Armadillo repeat protein (e.g., β-catenin, α-importin, plakoglobin, adenomatous polyposis coli (APC)), which comprises armadillo repeat units (characteristic, repetitive amino acid sequence of about 40 residues in length). Each Armadillo repeat is composed of a pair of alpha helices that form a hairpin structure. Multiple copies of the repeat form what is known as an alpha solenoid structure. Armadillo repeat proteins are able to bind different types of peptides, relying on a constant way of binding of the peptide backbone without requiring specific conserved side chains or interactions with free N- or C-termini of a peptide. The possibility of recognizing a peptide residue by residue, combined with the intrinsic modularity of a repeat protein, makes the armadillo repeat proteins promising candidates for the design of a generic scaffold for peptide binding.

### III. Vectors Encoding Long-Acting NGF Polypeptides

The present invention also provides isolated nucleic acids encoding any of the long-acting NGF polypeptides described herein, vectors comprising nucleic acids encoding any of the long-acting NGF polypeptides described herein. Also provided are isolated host cells (e.g., CHO cells, HEK 293 cells, Hela cells, or COS cells) comprising nucleic acids encoding any of the long-acting NGF polypeptides described herein, or vectors comprising nucleic acids encoding any of the long-acting NGF polypeptides described herein. In some embodiments, the isolated nucleic acid further encodes a signal peptide sequence (e.g., SEQ ID NO: 6) at the N-terminus of the long-acting NGF polypeptide. In some embodiments, the isolated nucleic acid further encodes a propeptide sequence (e.g., SEQ ID NO: 5) at the N-terminus of the long-acting NGF polypeptide. In some embodiments, the isolated nucleic acid further encodes a signal peptide sequence (e.g., SEQ ID NO: 6) followed by a propeptide sequence (e.g., SEQ ID NO: 5) at the N-terminus of the long-acting NGF polypeptide.

Thus in some embodiments, there is provided an isolated nucleic acid encoding a long-acting NGF polypeptide comprising from N-terminus to C-terminus an NGF moiety and an Fc moiety, wherein the NGF moiety comprises (or consists essentially of, or consists of) the amino acid sequence of any one of SEQ ID NOs: 1-4 (e.g., any of SEQ ID NOs: 1-3), and wherein the Fc moiety is derived from an IgG1 Fc or an IgG4 Fc. In some embodiments, there is provided an isolated nucleic acid encoding a long-acting NGF polypeptide comprising (or consisting essentially of, or consisting of) the amino acid sequence of any one of SEQ ID NOs: 61-67. In some embodiments, the isolated nucleic acid further comprises a nucleic acid sequence encoding the signal peptide sequence of SEQ ID NO: 6 at the 5′ end. In some embodiments, the isolated nucleic acid further comprises a nucleic acid sequence encoding the propeptide sequence of SEQ ID NO: 5 at the 5′ end. In some embodiments, the isolated nucleic acid further comprises (from 5′ to 3′) a nucleic acid sequence encoding the signal peptide sequence of SEQ ID NO: 6 followed by a nucleic acid sequence encoding the propeptide sequence of SEQ ID NO: 5 at the 5′ end.

In some embodiments, there is provided an isolated nucleic acid encoding a long-acting NGF polypeptide comprising (or consisting essentially of, or consisting of) the amino acid sequence of any of SEQ ID NOs: 34, 36, 38, 40, 42, 44, and 46. In some embodiments, there is provided an isolated nucleic acid encoding a long-acting NGF polypeptide comprising (or consisting essentially of, or consisting of) the amino acid sequence of any of SEQ ID NOs: 34, 36, 38, 40, 42, 44, and 46, excluding the signal peptide sequence of SEQ ID NO: 6. In some embodiments, there is provided an isolated nucleic acid comprising (or consisting essentially of, or consisting of) the nucleic acid sequence of any of SEQ ID NOS: 33, 35, 37, 39, 41, 43, and 45.

In some embodiments, the vector comprising a nucleic acid encoding any of the long-acting NGF polypeptides described herein is suitable for replication and integration in eukaryotic cells, such as mammalian cells (e.g., CHO cells, HEK 293 cells, Hela cells, COS cells). In some embodiments, the vector is a viral vector. In some embodiments, the vector is a non-viral vector, such as pTT5.

A number of viral based systems have been developed for gene transfer into mammalian cells. Examples of viral vectors include, but are not limited to, adenoviral vectors, adeno-associated virus vectors, lentiviral vector, retroviral vectors, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals. Retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the construct protein coding sequence(s) can be packaged with protocols known in the art. The resulting lentiviral vectors can be used to transduce a mammalian cell using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector is a non-viral vector. In some embodiments, the vector is a pTT5 vector. In some embodiments, the vector is a transposon, such as a Sleeping Beauty (SB) transposon system, or a PiggyBac transposon system. In some embodiments, the vector is a polymer-based non-viral vector, including for example, poly (lactic-co-glycolic acid) (PLGA) and poly lactic acid (PLA), poly (ethylene imine) (PEI), and dendrimers. In some embodiments, the vector is a cationic-lipid based non-viral vector, such as cationic liposome, lipid nanoemulsion, and solid lipid nanoparticle (SLN). In some embodiments, the vector is a peptide-based gene non-viral vector, such as Poly-L-lysine. Any of the known non-viral vectors suitable for genome editing can be used for introducing the long-acting NGF polypeptide-encoding nucleic acid(s) to the host cells. See, for example, Yin H. et al., Nature Rev. Genetics (2014) 15:521-555; Aronovich E L et al. “The Sleeping Beauty transposon system: a non-viral vector for gene therapy.” Hum. Mol. Genet. (2011) R1: R14-20; and Zhao S. et al. “PiggyBac transposon vectors: the tools of the human gene editing.” Transl. Lung Cancer Res. (2016) 5 (1): 120-125, which are incorporated herein by reference. In some embodiments, any one or more of the nucleic acids or vectors encoding the long-acting NGF polypeptides described herein is introduced to the host cells (e.g., CHO, HEK 293, Hela, or COS) by a physical method, including, but not limited to electroporation, sonoporation, photoporation, magnetofection, hydroporation.

In some embodiments, the vector contains a selectable marker gene or a reporter gene to select cells expressing the long-acting NGF polypeptides described herein from the population of host cells transfected through vectors (e.g., lentiviral vectors, pTT5 vectors). Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in prokaryotic cells or eukaryotic cells (e.g., mammalian cells), and any of the promoters known in the art may be used in the present invention. Promoters may be roughly categorized as constitutive promoters or regulated promoters, such as inducible promoters.

In some embodiments, the nucleic acid encoding the long-acting NGF polypeptides described herein is operably linked to a constitutive promoter. Constitutive promoters allow heterologous genes (also referred to as transgenes) to be expressed constitutively in the host cells. Exemplary promoters contemplated herein include, but are not limited to, cytomegalovirus immediate-early promoter (CMV), human elongation factors-1alpha (hEF1α), ubiquitin C promoter (UbiC), phosphoglycerokinase promoter (PGK), simian virus 40 early promoter (SV40), chicken β-Actin promoter coupled with CMV early enhancer (CAGG), a Rous Sarcoma Virus (RSV) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter. The efficiencies of such constitutive promoters on driving transgene expression have been widely compared in a huge number of studies. In some embodiments, the nucleic acid encoding the long-acting NGF polypeptides described herein is operably linked to CMV promoter.

In some embodiments, the nucleic acid encoding the long-acting NGF polypeptides described herein is operably linked to an inducible promoter. Inducible promoters belong to the category of regulated promoters. The inducible promoter can be induced by one or more conditions, such as a physical condition, microenvironment of the host cells, or the physiological state of the host cells, an inducer (i.e., an inducing agent), or a combination thereof. In some embodiments, the inducing condition does not induce the expression of endogenous genes in the host cell. In some embodiments, the inducing condition is selected from the group consisting of: inducer, irradiation (such as ionizing radiation, light), temperature (such as heat), redox state, and the activation state of the host cell. In some embodiments, the inducible promoter can be an NFAT promoter, a TETON® promoter, or an NFκB promoter.

### IV. Methods of Preparation

Also provided are methods of preparing any of the long-acting NGF polypeptides described herein. Thus in some embodiments, there is provided a method of producing a long-acting NGF polypeptide, comprising: (a) culturing a host cell (e.g., CHO cell, HEK 293 cell, Hela cell, or COS cell) comprising any of the nucleic acids or vectors encoding the long-acting NGF polypeptides described herein under a condition effective to express the encoded long-acting NGF polypeptides; and (b) obtaining the expressed long-acting NGF polypeptide from said host cell. In some embodiments, the method of step (a) further comprises producing a host cell comprising the nucleic acid or vector encoding the long-acting NGF polypeptide described herein. The long-acting NGF polypeptides described herein may be prepared using any methods known in the art or as described herein. Also see Example 1 for exemplary method.

In some embodiments, the long-acting NGF polypeptides described herein are expressed with eukaryotic cells, such as mammalian cells. In some embodiments, the long-acting NGF polypeptides described herein are expressed with prokaryotic cells. When long-acting NGF polypeptides described herein are expressed with prokaryotic cells, the produced proNGF-(optional linker)-Fc moiety cannot be processed for the propeptide sequence. Hence, nucleic acid encoding the long-acting NGF polypeptide when used for prokaryotic cell expression can be designed without the nucleic acid sequence encoding the propeptide sequence (e.g., SEQ ID NO: 5).

### 1. Recombinant Production in Prokaryotic Cells

### a) Vector Construction

Polynucleic acid sequences encoding the protein constructs of the present application can be obtained using standard recombinant techniques. Polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector, which can be used to transform susceptible host cells such as E. coli LE392.

A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the polypeptide by removing the promoter from the source DNA via restriction enzyme digestion and inserting the isolated promoter sequence into the vector of the present application. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the—galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleic acid sequences have been published, thereby enabling a skilled worker operably to ligate them to cistrons encoding the target light and heavy chains (Siebenlist et al. (1980) Cell 20:269) using linkers or adaptors to supply any required restriction sites.

In some embodiments, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP.

In some embodiments, the production of the protein construct according to the present application can occur in the cytoplasm of the host cell, and therefore does not require the presence of secretion signal sequences within each cistron. In some embodiments, polypeptide components are expressed, folded, and assembled to form the protein construct within the cytoplasm. Certain host strains (e.g., the E. coli trxB⁻ strains) provide cytoplasm conditions that are favorable for disulfide bond formation, thereby permitting proper folding and assembly of expressed protein subunits. See Proba and Pluckthun, Gene, 159:203 (1995).

### b) Prokaryotic Host Cells

Prokaryotic host cells suitable for expressing the proteins of the present application include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In some embodiments, E. coli cells are used as hosts for the invention. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A (nmpc-fepE) degP41 kan^{R} (U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well-known plasmids such as pBR322, pBR325, pACYC177, or pKN410 are used to supply the replicon.

Typically, the host cell should secrete minimal amounts of proteolytic enzymes, and additional protease inhibitors may desirably be incorporated in the cell culture.

### c) Protein Production

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the protein constructs of the present application are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol. The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the present application, protein expression is induced under conditions suitable for the activation of the promoter. In one aspect of the present application, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods (2002), 263:133-147). A variety of other inducers may be used, according to the vector construct employed, as is known in the art.

The expressed protein constructs of the present application are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small-scale fermentation refers generally to fermentation in a fermentor that is no more than approximately 100 liters in volumetric capacity, and can range from about 1 liter to about 100 liters.

During the fermentation process, induction of protein expression is typically initiated after the cells have been grown under suitable conditions to a desired density, e.g., an OD₅₅₀ of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the protein constructs of the present application, various fermentation conditions can be modified. For example, to improve the proper assembly and folding of the secreted polypeptides, additional vectors overexpressing chaperone proteins, such as Dsb proteins (DsbA, DsbB, DsbC, DsbD, or DsbG) or FkpA (a peptidylprolyl cis-, trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

E. coli strains deficient for proteolytic enzymes and transformed with plasmids overexpressing one or more chaperone proteins may be used as host cells in the expression system encoding the protein constructs of the present application.

### d) Protein Purification

The protein constructs produced herein are further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In some embodiments, Protein A immobilized on a solid phase is used for immunoaffinity purification of the protein constructs comprising an Fc region of the present application. Protein A is a 42 kDa surface protein from Staphylococcus aureas which binds with a high affinity to Fc-containing constructs, e.g., long-acting NGF polypeptides described herein. Lindmark et al (1983) J. Immunol. Meth. 62:1-13. The solid phase to which Protein A is immobilized is preferably a column comprising a glass or silica surface, more preferably a controlled pore glass column or a silicic acid column. In some applications, the column has been coated with a reagent, such as glycerol, in an attempt to prevent nonspecific adherence of contaminants. The solid phase is then washed to remove contaminants non-specifically bound to the solid phase. Finally, the protein constructs of interest are recovered from the solid phase by elution.

### 2. Recombinant Production in Eukaryotic Cells

For eukaryotic expression, the vector components generally include, but are not limited to, one or more of the following, a signal sequence, an origin of replication, one or more marker genes, and enhancer element, a promoter, and a transcription termination sequence.

### a) Signal Sequence Component

A vector for use in a eukaryotic host may also an insert that encodes a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available. The DNA for such precursor region is ligated in reading frame to DNA encoding the protein constructs of the present application.

### b) Origin of Replication

Generally, the origin of replication component is not needed for mammalian expression vectors (the SV40 origin may typically be used only because it contains the early promoter).

### c) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up nucleic acid encoding the protein constructs of the present application, such as DHFR, thymidine kinase, metallothionein-I and —II, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with the polypeptide encoding-DNA sequences, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

### d) Promoter Component

Expression and cloning vectors usually contain a promoter that is recognized by the host organism and is operably linked to the nucleic acid encoding the desired polypeptide sequences. Virtually all eukaryotic genes have an AT-rich region located approximately 25 to 30 based upstream from the site where transcription is initiated. Another sequence found 70 to 80 bases upstream from the start of the transcription of many genes is a CNCAAT region where N may be any nucleotide. At the 3′ end of most eukaryotic is an AATAAA sequence that may be the signal for addition of the poly A tail to the 3′ end of the coding sequence. All of these sequences may be inserted into eukaryotic expression vectors. Also see section “III. Vectors encoding long-acting NGF polypeptides” above.

Polypeptide transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human-interferon cDNA in mouse cells under the control of a thymidine kinase promoter from herpes simplex virus. Alternatively, the Rous Sarcoma Virus long terminal repeat can be used as the promoter.

### e) Enhancer Element Component

Transcription of a DNA encoding the protein constructs of the present application by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (100-270 bp), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide encoding sequence, but is preferably located at a site 5′ from the promoter.

### f) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA. One useful transcription termination component is the bovine growth hormone polyadenylation region. See WO94/11026 and the expression vector disclosed therein.

### g) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); COS fibroblast-like cell lines derived from monkey kidney tissue; human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for protein construct production and cultured in conventional nutrient media modified as appropriate for inducing promoters, selecting transformants, or amplifying the genes encoding the desired sequences.

### h) Culturing the Host Cells

The host cells used to produce the protein constructs of the present application may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### i) Protein Purification

When using recombinant techniques, the protein constructs of the present invention can be produced intracellularly, in the periplasmic space, or directly secreted into the medium. If the protein construct is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the protein construct is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the Fc-containing protein construct. Protein A can be used to purify Fc-containing proteins based on human immunoglobulins containing 1, 2, or 4 heavy chains (Lindmark et al., J. Immunol. Meth. 62:1-13 (1983)). Protein G is recommended for all mouse isotypes and for human 3 (Guss et al., EMBO J. 5:15671575 (1986)). The matrix to which the affinity ligand is attached is most often agarose, but other matrices are available. Mechanically stable matrices such as controlled pore glass or poly (styrene-divinyl) benzene allow for faster flow rates and shorter processing times than can be achieved with agarose. Where the protein construct comprises a C_{H}3 domain, the Bakerbond ABXTMresin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the protein construct to be recovered.

Following any preliminary purification step(s), the mixture comprising the protein constructs of interest and contaminants may be subjected to low pH hydrophobic interaction chromatography using an elution buffer at a pH between about 2.5-4.5, preferably performed at low salt concentrations (e.g., from about 0-0.25M salt).

### V. Pharmaceutical Compositions

Further provided are pharmaceutical compositions comprising any of the long-acting NGF polypeptides described herein, and optionally a pharmaceutically acceptable carrier. Thus in some embodiments, there is provided a pharmaceutical composition comprising a long-acting NGF polypeptide comprising from N-terminus to C-terminus an NGF moiety and an Fc moiety, wherein the NGF moiety comprises the amino acid sequence of any one of SEQ ID NOs: 1-4, and wherein the Fc moiety is derived from an IgG1 Fc or an IgG4 Fc, and optionally a pharmaceutically acceptable carrier. Pharmaceutical compositions can be prepared by mixing a long-acting NGF polypeptide described herein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

A reconstituted formulation can be prepared by dissolving a lyophilized long-acting NGF polypeptide in a diluent such that the protein is dispersed throughout. Exemplary pharmaceutically acceptable (safe and non-toxic for administration to a human) diluents suitable for use in the present application include, but are not limited to, sterile water, bacteriostatic water for injection (BWFI), a pH buffered solution (e.g. phosphate-buffered saline), sterile saline solution, Ringer's solution or dextrose solution, or aqueous solutions of salts and/or buffers.

In some embodiments, the pharmaceutical composition comprises a homogeneous population of long-acting NGF polypeptides described herein. A homogeneous population means the long-acting NGF polypeptides are exactly the same to each other, e.g., same long-acting NGF polypeptide configuration, same NGF moiety, same linker if any, and same Fc moiety. In some embodiments, at least about 70% (such as at least about any of 75%, 80%, 85%, 90%, 95%, 96%, 97%, 98%, or 99%) of the long-acting NGF polypeptides in the pharmaceutical composition are homogeneous.

In some embodiments, the pharmaceutical composition consists essentially of (e.g., consists of) long-acting NGF polypeptides described herein, and optionally a pharmaceutically acceptable carrier. In some embodiments, the pharmaceutical composition does not comprise any proNGF-Fc or preproNGF-Fc fusion protein. In some embodiments, the pharmaceutical composition comprises at most about 5% (such as at most about any of 4%, 3%, 2%, or 1%) proNGF-Fc or preproNGF-Fc fusion protein. In some embodiments, the pharmaceutical composition does not comprise any host cell (e.g., CHO) protein.

The pharmaceutical composition is preferably to be stable, in which the proteins contained within essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month, and/or stable at 2-8° C. for at least 2 years. Where the formulation is to be stored at 30° C., generally the formulation should be stable for at least 2 years at 30° C., and/or stable at 40° C. for at least 6 months. For example, the extent of aggregation during storage can be used as an indicator of protein stability. In some embodiments, the stable formulation of long-acting NGF polypeptides described herein may comprise less than about 10% (preferably less than about 5%) of the long-acting NGF polypeptide present as an aggregate in the formulation.

In some embodiments, the pharmaceutical composition has a shelf life of at least about 15 days, such as at least about any of 20 days, 1 month, 2 months, 3 months, 6 months, 1 year, 2 years, 3 years, or longer, for example, at about 2-25° C., such as about 2-8° C. As used herein, “shelf life” means that the storage period during which an active ingredient such as a therapeutic protein (e.g., the long-acting NGF polypeptides described herein) in a pharmaceutical formulation has minimal degradation (e.g., not more than about 5% degradation, such as not more than about 4%, 3%, or 2% degradation) when the pharmaceutical formulation is stored under specified storage conditions, for example, 2-8° C. Exemplary techniques for assessing protein or formulation stability include size-exclusion chromatography (SEC)-HPLC to detect, e.g., aggregation, reverse phase (RP)-HPLC to detect, e.g. protein fragmentation, ion exchange-HPLC to detect, e.g., changes in the charge of the protein, mass spectrometry, fluorescence spectroscopy, circular dichroism (CD) spectroscopy, Fourier transform infrared spectroscopy (FT-IR), and Raman spectroscopy to detect protein conformational changes. All of these techniques can be used singly or in combination to assess the degradation of the protein in the pharmaceutical formulation and determine the shelf life of that formulation. In some embodiments, the pharmaceutical formulations of the present invention exhibit degradation (e.g., fragmentation, aggregation, or unfolding) of not more than about 5% (e.g., not more than about 4%, 3%, 2%, or 1%) for at least about 15 days (e.g., at least about any of 20 days, 1 month, 2 months, 3 months, 6 months, 1 year, 2 years, 3 years, or longer) when stored at about 2-8° C.

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers (e.g. sodium chloride), stabilizers, metal complexes (e.g. Zn-protein complexes); chelating agents such as EDTA and/or non-ionic surfactants.

Examples of physiologically acceptable carriers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or nonionic surfactants such as TWEEN™, polyethylene glycol (PEG), and PLURONICS™ or polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use in the present application include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). The addition of a preservative may, for example, facilitate the production of a multi-use (multiple-dose) formulation. Suitable preservatives for use in the present application include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1% to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) are present to help solubilize the proteins as well as to protect the proteins against agitation-induced aggregation, which also permits the formulation to be exposed to shear surface stress without causing denaturation of the active proteins. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

Sustained-release preparations may be prepared. Suitable examples of sustained-release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly (2-hydroxyethyl-methacrylate), or poly (vinylalcohol)), polylactides (U.S. Pat. No. 3,773,919), copolymers of L-glutamic acid and Ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The pharmaceutical compositions herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

### VI. Methods of Treating Diseases

The long-acting NGF polypeptides described herein and compositions (e.g., pharmaceutical compositions) thereof are useful for a variety of applications, such as in diagnosis, molecular assays, and therapy. In some embodiments, there is provided a method of treating a disease (e.g., NGF-related disease such as neurological disease) in an individual (e.g., human), comprising administering to the individual an effective amount of any of the long-acting NGF polypeptides described herein or pharmaceutical compositions thereof. The term “NGF-related disease” used herein refers to any disease or disorder caused by or associated with impaired NGF receptor signaling (such as due to insufficient NGF amount and/or reduced binding affinity), or disease or disorder that requires NGF bioactivities for treatment (such as injury/damage that requires growth, maintenance, proliferation, and/or survival of neurons for treatment). In some embodiments, the long-acting NGF polypeptide (or pharmaceutical composition thereof) is administered intravenously, intramuscularly, or subcutaneously.

Thus in some embodiments, there is provided a method of treating a disease (e.g., NGF-related disease, such as neurological disease (e.g., diabetic neuropathy, Alzheimer's disease, or neurotrophic keratitis) or non-neurological disease (e.g., premature ovarian failure or spermatogenesis disorder)) in an individual (e.g., human), comprising administering to the individual an effective amount of a long-acting NGF polypeptide (or pharmaceutical composition thereof) comprising from N-terminus to C-terminus an NGF moiety and an Fc moiety, wherein the NGF moiety comprises (or consists essentially of, or consists of) the amino acid sequence of any one of SEQ ID NOs: 1-4 (e.g., any of SEQ ID NOs: 1-3), and wherein the Fc moiety is derived from an IgG1 Fc or an IgG4 Fc. In some embodiments, there is provided a method of treating a disease (e.g., NGF-related disease, such as neurological disease (e.g., diabetic neuropathy, Alzheimer's disease, or neurotrophic keratitis) or non-neurological disease (e.g., premature ovarian failure or spermatogenesis disorder)) in an individual (e.g., human), comprising administering to the individual an effective amount of a long-acting NGF polypeptide (or pharmaceutical composition thereof) comprising (or consisting essentially of, or consisting of) the amino acid sequence of any one of SEQ ID NOs: 61-67. In some embodiments, the long-acting NGF polypeptide (or pharmaceutical composition thereof) is administered intravenously, intramuscularly, or subcutaneously.

The methods described herein are suitable for treating both neurological diseases and non-neurological diseases.

Neurological diseases comprise nervous system diseases. Nervous system disease refers to a disease associated with neuronal degeneration or injury in the central and/or peripheral nervous system. Specific examples of nervous system diseases include, but are not limited to, Alzheimer's disease, Parkinson's disease, Huntington's disease, stroke, amyotrophic lateral sclerosis (ALS), facial neuritis, craniocerebral or spinal cord trauma, acute cerebrovascular disease, brain atrophy, peripheral neuropathy, and other disorders characterized by necrosis or loss of neuron, regardless of central neuron, peripheral neuron, or motor neuron, besides nerve damage caused by trauma, burns, kidney failure, injury, or chemicals/drugs, such as acute cerebrovascular central nerve damage caused by chemicals or drugs. Nervous system diseases also include peripheral neuropathy associated with certain conditions, such as a neuropathy associated with diabetes, AIDS, or chemotherapy. In some embodiments, the neurological disease is selected from the group consisting of multi-infarct dementia, vascular dementia, cognitive impairment due to organic brain disease due to alcoholism, Alzheimer's disease, Parkinson's disease, epilepsy, multiple sclerosis, Huntington's chorea, Down's syndrome, nerve deafness, Meniere's disease, stroke, ALS, Bell's palsy, conditions involving spinal muscular atrophy, conditions involving paralysis, peripheral neuropathies, neural damage due to trauma, neural damage due to burns, neural damage due to kidney dysfunction, neural damage due to injury, neural damage due to toxic effects of chemotherapy, neural damage due to surgery, neural damage due to ischemia, neural damage due to infection, neural damage due to metabolic disease, and neural damage due to nutritional deficiency. In some embodiments, the neurological disease is a peripheral neuropathy selected from the group consisting of diabetic peripheral neuropathy, toxin-induced peripheral neuropathy, chemotherapy-induced peripheral neuropathy, HIV-associated peripheral neuropathy, and a peripheral neuropathy that affects motoneurons. In some embodiments, the neurological disease is selected from the group consisting of neonatal hypoxic-ischemic encephalopathy, cerebral palsy, critical illness myopathy, nerve deafness, recurrent laryngeal nerve injury, traumatic brain injury, tooth nerve injury, cerebral stroke, Down syndrome, ALS, multiple sclerosis, spinal muscular atrophy, diffuse brain injury, thymus dysplasia, optic contusion, follicular dysplasia, spinal cord injury, glaucoma, neurotrophic keratitis, optic injury, neuromyelitis optica, retinal associated diseases, urinary incontinence, Alzheimer's disease, Parkinson's disease, Huntington's disease, dementia, Hypertensive Intracerebral Hemorrhage Neurological Dysfunction, Cerebral Small Vessel Disease, Acute Ischemic Stroke, corneal endothelial dystrophy, diabetic neuropathy, diabetic foot ulcer, neurogenic skin ulcer, pressure sore, neurotrophic corneal ulcer, diabetic corneal ulcer, and macular hole.

Non-neurological diseases can include atrophy of spleen, contusion of spleen, diminished ovarian reserve, premature ovarian failure (POF), Ovarian Hyperstimulation Syndrome, ovarian remnant syndrome, ovarian follicular dysplasia, spermatogenesis disorder (e.g., oligozoospermia (or oligospermia), asthenospermia, oligoasthenospermia, azoospermia, teratozoospermia, oligoasthenoteratozoospermia (OAT syndrome)), ischemic ulcer, stress ulcer, rheumatoid ulcer, liver fibrosis, corneal ulcer, burns, oral ulcer, and venous leg ulcers.

In some embodiments, the method of treating a disease (e.g., NGF-related disease, such as neurological disease (e.g., diabetic neuropathy, Alzheimer's disease, or neurotrophic keratitis) or non-neurological disease (e.g., premature ovarian failure or spermatogenesis disorder)) has one or more of the following biological activities: (i) supporting neuronal survival; (ii) promoting neurite outgrowth; (iii) enhancing neurochemical differentiation; (iv) promoting the proliferation of pancreatic β cells; (v) inducing innate and/or acquired immunity; (vi) restoring damaged nerve cells (e.g., cornea corneal nerve) and/or preventing damage (e.g., in neurotrophic keratitis); (vii) promoting the proliferation and/or estrogen secretion of ovarian follicle cells; (viii) promoting wound healing (e.g., in diabetic having neurodegenerative disease (e.g., Alzheimer's disease); (x) treating and/or preventing neurodegeneration; (xi) treating testicular seminiferous tubule atrophy, seminiferous tubule spermatogenesis disorder, and/or epididymal duct cell fragments; (xii) rescuing the reduction of sperm number and/or motility, or increasing sperm number and/or motility (e.g., in spermatogenic disorder); (xiii) preventing/reversing the reduction of the number and/or function of ovarian follicles, or increasing the number and/or function of ovarian follicles (e.g., in premature ovarian failure); and/or (xiv) prolonging patient survival. In some embodiments, the method of supporting neuronal survival mediated by the long-acting NGF polypeptide or pharmaceutical composition described herein can achieve neuronal survival rate of at least about any of 40%, 50%, 60%, 70%, 80%, 90%, 95%, or more. In some embodiments, the method of promoting neurite outgrowth mediated by the long-acting NGF polypeptide or pharmaceutical composition described herein can promote at least about 2 folds (including for example at least about any of 3, 4, 5, 6, 7, 8, 9, 10, 20, 30, 40, or 50 folds, or more) of neurite outgrowth. In some embodiments, the method of enhancing neurochemical differentiation mediated by the long-acting NGF polypeptide or pharmaceutical composition described herein can enhance at least about 2 folds (including for example at least about any of 3, 4, 5, 6, 7, 8, 9, 10, 20, 30, 40, or 50 folds, or more) of neurochemical differentiation. In some embodiments, the method of promoting the proliferation of pancreatic β cells mediated by the long-acting NGF polypeptide or pharmaceutical composition described herein can promote at least about 2 folds (including for example at least about any of 3, 4, 5, 6, 7, 8, 9, 10, 20, 30, 40, or 50 folds, or more) of pancreatic β cell proliferation. In some embodiments, the method of inducing ovulation mediated by the long-acting NGF polypeptide or pharmaceutical composition described herein can enhance at least about 2 folds (including for example at least about any of 3, 4, 5, 6, 7, 8, 9, 10, 20, 30, 40, or 50 folds, or more) of ovulation. In some embodiments, the method of inducing innate and/or acquired immunity mediated by the long-acting NGF polypeptide or pharmaceutical composition described herein can induce at least about 1.1 folds (including for example at least about any of 1.2, 1.3, 1.4, 1.5, 1.6, 1.7, 1.8, 1.9, 2, 3, 4, 5, 6, 7, 8, 9, 10, 20, 30, 40, or 50 folds, or more) of innate and/or acquired immunity. In some embodiments, the method of rescuing and/or preventing neuronal damage mediated by the long-acting NGF polypeptide or pharmaceutical composition described herein can rescue and/or prevent at least about 5% (including for example at least about any of 10%, 20%, 30%, 40%, 50%, 60%, 70%, 80%, 90%, 95%, or more) of neuronal damage, or have a rescue and/or prevention efficacy of at least about 1.1 folds (including for example at least about any of 1.2, 1.3, 1.4, 1.5, 1.6, 1.7, 1.8, 1.9, 2, 3, 4, 5, 6, 7, 8, 9, 10, 20, 30, 40, or 50 folds, or more). In some embodiments, the method of promoting the proliferation and/or estrogen secretion of ovarian follicle cells mediated by the long-acting NGF polypeptide or pharmaceutical composition described herein can promote at least about 1.1 folds (including for example at least about any of 1.2, 1.3, 1.4, 1.5, 1.6, 1.7, 1.8, 1.9, 2, 3, 4, 5, 6, 7, 8, 9, 10, 20, 30, 40, or 50 folds, or more) of proliferation and/or estrogen secretion of ovarian follicle cells. In some embodiments, the method of promoting wound healing mediated by the long-acting NGF polypeptide or pharmaceutical composition described herein can promote at least about 1.1 folds (including for example at least about any of 1.2, 1.3, 1.4, 1.5, 1.6, 1.7, 1.8, 1.9, 2, 3, 4, 5, 6, 7, 8, 9, 10, 20, 30, 40, or 50 folds, or more) of wound healing. In some embodiments, the method of improving spatial cognition, memory, and/or learning ability of a subject having a neurodegenerative disease (e.g., Alzheimer's disease) mediated by the long-acting NGF polypeptide or pharmaceutical composition described herein can improve at least about 1.1 folds (including for example at least about any of 1.2, 1.3, 1.4, 1.5, 1.6, 1.7, 1.8, 1.9, 2, 3, 4, 5, 6, 7, 8, 9, 10, 20, 30, 40, or 50 folds, or more) of spatial cognition, memory, and/or learning ability. In some embodiments, the method of treating and/or preventing neurodegeneration mediated by the long-acting NGF polypeptide or pharmaceutical composition described herein can rescue and/or prevent at least about 5% (including for example at least about any of 10%, 20%, 30%, 40%, 50%, 60%, 70%, 80%, 90%, 95%, or more) of neurodegeneration. In some embodiments, the method of treating testicular seminiferous tubule atrophy, seminiferous tubule spermatogenesis disorder, and/or epididymal duct cell fragments mediated by the long-acting NGF polypeptide or pharmaceutical composition described herein can treat at least about 5% (including for example at least about any of 10%, 20%, 30%, 40%, 50%, 60%, 70%, 80%, 90%, 95%, or more) of testicular seminiferous tubule atrophy, seminiferous tubule spermatogenesis disorder, and/or epididymal duct cell fragments. In some embodiments, the method of rescuing the reduction of sperm number and/or motility mediated by the long-acting NGF polypeptide or pharmaceutical composition described herein can rescue at least about 5% (including for example at least about any of 10%, 20%, 30%, 40%, 50%, 60%, 70%, 80%, 90%, 95%, or more) of the reduction of sperm number and/or motility. In some embodiments, the method of increasing sperm number and/or motility mediated by the long-acting NGF polypeptide or pharmaceutical composition described herein can increase at least about 5% (including for example at least about any of 10%, 20%, 30%, 40%, 50%, 60%, 70%, 80%, 90%, 100%, 150%, 200%, or more) of sperm number and/or motility. In some embodiments, the method of rescuing the reduction of the number and/or function of ovarian follicles mediated by the long-acting NGF polypeptide or pharmaceutical composition described herein can rescue at least about 5% (including for example at least about any of 10%, 20%, 30%, 40%, 50%, 60%, 70%, 80%, 90%, 95%, or more) of the reduction of the number and/or function of ovarian follicles. In some embodiments, the method of increasing the number and/or function of ovarian follicles mediated by the long-acting NGF polypeptide or pharmaceutical composition described herein can increase at least about 5% (including for example at least about any of 10%, 20%, 30%, 40%, 50%, 60%, 70%, 80%, 90%, 100%, 150%, 200%, or more) of the number and/or function of ovarian follicles. In some embodiments, the method of prolonging survival of an individual (e.g., human) mediated by the long-acting NGF polypeptide or pharmaceutical composition described herein can prolong the survival of the individual by at least any of 1, 2, 3, 4, 5, 6, 7, 8, 9, 10, 11, 12, 18, 24 months, or 2, 3, 4, 5, 6, 7, 8, 9, 10 years, or longer.

Administration of the long-acting NGF polypeptides described herein or pharmaceutical compositions thereof may be carried out in any convenient manner, including by injection or transfusion. The route of administration is in accordance with known and accepted methods, such as by single or multiple bolus or infusion over a long period of time in a suitable manner. The long-acting NGF polypeptides or pharmaceutical compositions thereof may be administered to a patient orally, subcutaneously, intravenously, intracerebrally, intranasally, transdermally, intraperitoneally, intramuscularly, intrapulmonarily, vaginally, rectally, intraocularly, topically, transarterially, intradermally, intranodally, intraputaminally, or intramedullary, intrathecally, intraventricularly, intracerebrally, intraspinally, intrathecially, ntralesionally, or intraocularly. In some embodiments, the long-acting NGF polypeptide or pharmaceutical composition thereof is administered systemically. In some embodiments, the long-acting NGF polypeptide or pharmaceutical composition thereof is administered to an individual by infusion, such as intravenous infusion. Infusion techniques for immunotherapy are known in the art (see, e.g., Rosenberg et al., New Eng. J. of Med. 319:1676 (1988)). In some embodiments, the long-acting NGF polypeptide or pharmaceutical composition thereof is administered to an individual by intradermal or subcutaneous (i.e. beneath the skin) injection. For subcutaneous injections, the long-acting NGF polypeptide or pharmaceutical composition thereof may be injected using a syringe. However, other devices for administration of the long-acting NGF polypeptide or pharmaceutical composition thereof are available such as injection devices; injector pens; auto-injector devices, needleless devices; and subcutaneous patch delivery systems. In some embodiments, the long-acting NGF polypeptide or pharmaceutical composition thereof is administered by intravenous injection. In some embodiments, the long-acting NGF polypeptide or pharmaceutical composition thereof is injected directly into the brain or spine. In some embodiments, the long-acting NGF polypeptide or pharmaceutical composition thereof is administered locally to a site of damage or injury, such as directly to wound tissue. In some embodiments, the long-acting NGF polypeptide or pharmaceutical composition thereof is administered by sustained release or extended-release means.

Dosages and desired drug concentration of pharmaceutical compositions of the present invention may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

When in vivo administration of the long-acting NGF polypeptide or pharmaceutical composition thereof are used, normal dosage amounts may vary from about 0.01 μg/kg to about 10 mg/kg of mammal body weight depending upon the route of administration and mammal type. It is within the scope of the present application that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays. In some embodiments, the long-acting NGF polypeptide or pharmaceutical composition thereof is administered in an amount of about 0.01 μg/kg to about 10 mg/kg, such as any of about 0.01 μg/kg to about 1 μg/kg, about 1 μg/kg to about 100 μg/kg, about 100 μg/kg to about 500 μg/kg, about 500 μg/kg to about 1 mg/kg, about 1 mg/kg to about 10 mg/kg, or about 0.01 μg/kg to about 1 mg/kg. In some embodiments, the long-acting NGF polypeptide or pharmaceutical composition thereof is administered at a dose of about 0.01 μg to about 1000 μg per individual (e.g., human), such as any of about 0.01 μg to about 1 μg, about 1 μg to about 500 μg, about 500 μg to about 1000 μg, about 1 μg to about 300 μg, or about 100 μg to about 1000 μg per individual.

In some embodiments, the long-acting NGF polypeptide or pharmaceutical composition thereof is administered for a single time (e.g. bolus injection). In some embodiments, the long-acting NGF polypeptide or pharmaceutical composition thereof is administered for multiple times (such as any of 2, 3, 4, 5, 6, or more times). If multiple administrations, they may be performed by the same or different routes and may take place at the same site or at alternative sites. The long-acting NGF polypeptide or pharmaceutical composition thereof may be administered daily to once per year. The interval between administrations can be about any one of 24 hours to a year. Intervals can also be irregular (e.g. following tumor progression). In some embodiments, there is no break in the dosing schedule. The optimal dosage and treatment regime for a particular patient can readily be determined by one skilled in the art of medicine by monitoring the patient for signs of disease and adjusting the treatment accordingly. In some embodiments, the long-acting NGF polypeptide described herein or pharmaceutical composition thereof is administered once per day (daily), once per 2 days, once per 3 days, once per 4 days, once per 5 days, once per 6 days, once per week, once per 10 days, once every 2 weeks, once every 3 weeks, once every 4 weeks, once per month, once per 2 months, once per 3 months, once per 4 months, once per 5 months, once per 6 months, once per 7 months, once per 8 months, once per 9 months, or once per year. In some embodiments, the long-acting NGF polypeptide or pharmaceutical composition thereof is administered once every three days. In some embodiments, the long-acting NGF polypeptide or pharmaceutical composition thereof is administered once every week. In some embodiments, the long-acting NGF polypeptide or pharmaceutical composition thereof is administered once per month. In some embodiments, the long-acting NGF polypeptide or pharmaceutical composition thereof is dropped once per day. In some embodiments, the long-acting NGF polypeptide or pharmaceutical composition thereof is dropped three times per day. In some embodiments, the long-acting NGF polypeptide or pharmaceutical composition thereof is dropped five times per day.

In some embodiments, the long-acting NGF polypeptide or pharmaceutical composition thereof is administered in split doses, such as about any one of 2, 3, 4, 5, or more doses. In some embodiments, the split doses are administered over about a week, a month, 2 months, 3 months, or longer. In some embodiments, the dose is equally split. In some embodiments, the split doses are about 20%, about 30% and about 50% of the total dose. In some embodiments, the interval between consecutive split doses is about 1 day, 2 days, 3 days, 1 week, 2 weeks, 3 weeks, a month, 3 months, 6 months, or longer. For repeated administrations over several days or longer, depending on the condition, the treatment is sustained until a desired suppression of disease symptoms occurs. However, other dosage regimens may be useful. The progress of this therapy is easily monitored by conventional techniques and assays.

### VII. Articles of Manufacture and Kits

Further provided are kits, unit dosages, and articles of manufacture comprising any of the long-acting NGF polypeptides described herein. In some embodiments, a kit is provided which contains any one of the pharmaceutical compositions described herein and preferably provides instructions for its use, such as for use in the treatment of the disorders described herein (e.g., neurological disease).

Kits of the invention include one or more containers comprising a long-acting NGF polypeptide described herein, e.g., for treating a disease. For example, the instructions comprise a description of administration of the long-acting NGF polypeptide to treat a disease, such as neurological disease. The kit may further comprise a description of selecting an individual (e.g., human) suitable for treatment based on identifying whether that individual has the disease and the stage of the disease. The instructions relating to the use of the long-acting NGF polypeptide generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The kits of the present application are in suitable packaging. Suitable packaging includes, but is not limited to, vials, bottles, jars, flexible packaging (e.g., sealed Mylar or plastic bags), and the like. Also contemplated are packages for use in combination with a specific device, such as an infusion device such as a minipump. A kit may have a sterile access port (for example, the container may be an intravenous solution bag or a vial having a stopper pierceable by a hypodermic injection needle). At least one active agent in the composition is a long-acting NGF polypeptide as described herein. The container may further comprise a second pharmaceutically active agent. The kits may optionally provide additional components such as buffers and interpretive information. Normally, the kit comprises a container and a label or package insert(s) on or associated with the container.

The present application thus also provides articles of manufacture, which include vials (such as sealed vials), bottles, jars, flexible packaging, and the like. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as neurological disease) described herein, and may have a sterile access port (for example, the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### EXAMPLES

The examples below are intended to be purely exemplary of the invention and should therefore not be considered to limit the invention in any way. The following examples and detailed description are offered by way of illustration and not by way of limitation.

### Example 1: Preparation of NGF Polypeptides

### Plasmid Construction

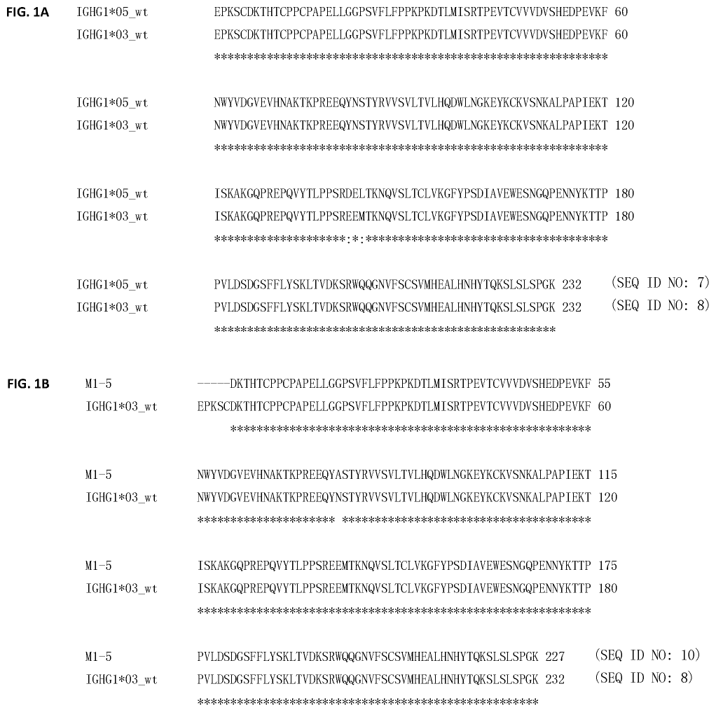

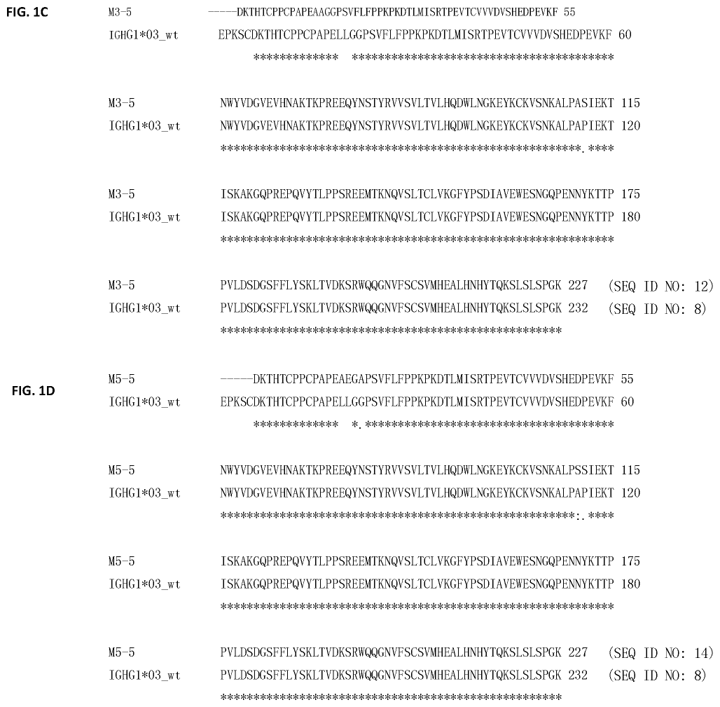

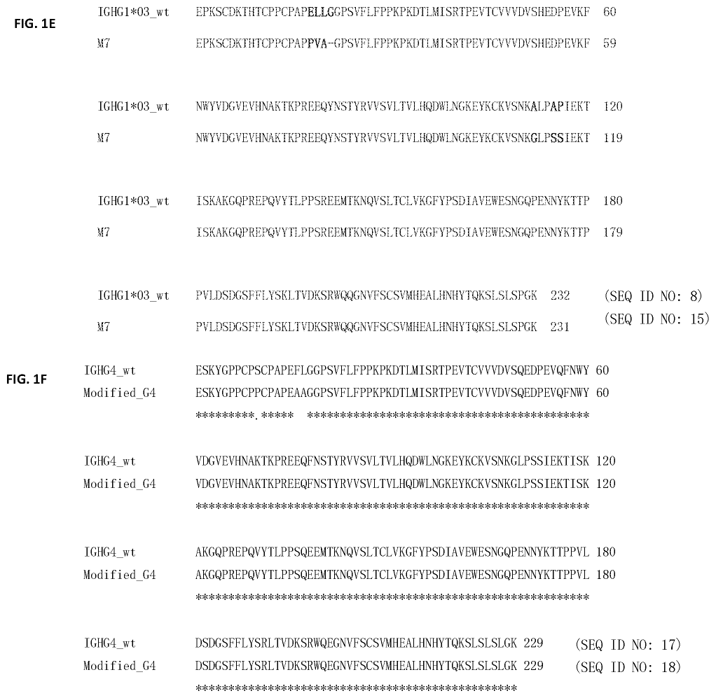

Plasmid construction is exemplified with preproNGF-Fc fusion protein 2-118-L3Fc10-M1-5 (SEQ ID NO: 34). Similar methods were used for other preproNGF-Fc fusion proteins, and control prepro-mNGF118 (SEQ ID NO: 47), which is a preproNGF with F12E mutation and two C-terminus amino acids (Arg-Ala) truncated in the β-NGF portion. FD-G4Fc (see CN105273087A) and WM-G24Fc (see CN106008722A) consturcts served as controls. NGF-1-15M7 (rhNGF-Fc1), NGF-L3Fc10M7-5 (rhNGF-Li-Fc1), 2-1-15M7 (rhNGF-(F12E)-Fc1), and NGF-4-12PAA (rhNGF-Fc4) were constructs as described in WO2017157325. See Table 1 for configurations of different NGF polypeptides, and FIGS. 1A-1F for Fc moiety alignments.

TABLE 1
Configurations of NGF polypeptides

| Construct | Mature β-NGF moiety | Linker | Fc moiety |
|---|---|---|---|
| mNGF118 | mutant β-NGF 118aa | / | |
| (prepro SEQ ID NO: 47) | (SEQ ID NO: 1) | | |
| (mature SEQ ID NO: 1) | | | |
| FD-G4Fc | wt β-NGF 118aa | GSGGGSGGGGS | modified IgG4 Fc-FD |
| (prepro SEQ ID NO: 22) | (SEQ ID NO: 3) | GGGGSGGGGS | (SEQ ID NO: 19) |
| (mature SEQ ID NO: 55) | | (SEQ ID NO: 71) | |
| WM-G24Fc | wt β-NGF 118aa | KTGGGSGGGS | modified IgG2/4 Fc |
| (prepro SEQ ID NO: 24) | (SEQ ID NO: 3) | (SEQ ID NO: 72) | (SEQ ID NO: 20) |
| (mature SEQ ID NO: 56) | | | |
| NGF-1-15M7 | wt β-NGF 120aa | / | IgG1 M7 |
| (rhNGF-Fc1) | (SEQ ID NO: 4) | | (SEQ ID NO: 15) |
| (prepro SEQ ID NO: 26) | | | |
| (mature SEQ ID NO: 57) | | | |
| NGF-L3Fc10M7-5 | wt β-NGF 120aa | (G4S)₃ | IgG1 M7-5 (N′ minus 5aa) |
| (rhNGF-Li-Fc1) | (SEQ ID NO: 4) | (SEQ ID NO: 68) | (SEQ ID NO: 16) |
| (prepro SEQ ID NO: 28) | | | |
| (mature SEQ ID NO: 58) | | | |
| 2-1-15M7 | mutant β-NGF 120aa | / | IgG1 M7 |
| (rhNGF-(F12E)-Fc1) | (SEQ ID NO: 2) | | (SEQ ID NO: 15) |
| (prepro SEQ ID NO: 30) | | | |
| (mature SEQ ID NO: 59) | | | |
| NGF-4-12PAA | wt β-NGF 120aa | / | modified IgG4 Fc |
| (rhNGF-Fc4) | (SEQ ID NO: 4) | | (SEQ ID NO: 18) |
| (prepro SEQ ID NO: 32) | | | |
| (mature SEQ ID NO: 60) | | | |
| 2-118-L3Fc10-M1-5 | mutant β-NGF 118aa | (G4S)₃ | IgG1 M1-5 (N′ minus 5aa) |
| (prepro SEQ ID NO: 34) | (SEQ ID NO: 1) | (SEQ ID NO: 68) | (SEQ ID NO: 10) |
| (mature SEQ ID NO: 61) | | | |
| 2-118-L3Fc10-M3-5 | mutant β-NGF 118aa | (G4S)₃ | IgG1 M3-5 (N′ minus 5aa) |
| (prepro SEQ ID NO: 36) | (SEQ ID NO: 1) | (SEQ ID NO: 68) | (SEQ ID NO: 12) |
| (mature SEQ ID NO: 62) | | | |
| NGF-118-L3Fc10-M3-5 | wt β-NGF 118aa | (G4S)₃ | IgG1 M3-5 (N′ minus 5aa) |
| (prepro SEQ ID NO: 38) | (SEQ ID NO: 3) | (SEQ ID NO: 68) | (SEQ ID NO: 12) |
| (mature SEQ ID NO: 63) | | | |
| 2-L3Fc10-M3-5 | mutant β-NGF 120aa | (G4S)₃ | IgG1 M3-5 (N′ minus 5aa) |
| (prepro SEQ ID NO: 40) | (SEQ ID NO: 2) | (SEQ ID NO: 68) | (SEQ ID NO: 12) |
| (mature SEQ ID NO: 64) | | | |
| 2-118-L3Fc10-M5-5 | mutant β-NGF 118aa | (G4S)₃ | IgG1 M5-5 (N′ minus 5aa) |
| (prepro SEQ ID NO: 42) | (SEQ ID NO: 1) | (SEQ ID NO: 68) | (SEQ ID NO: 14) |
| (mature SEQ ID NO: 65) | | | |
| 2-118-L3Fc10M7-5 | mutant β-NGF 118aa | (G4S)₃ | IgG1 M7-5 (N′ minus 5aa) |
| (prepro SEQ ID NO: 44) | (SEQ ID NO: 1) | (SEQ ID NO: 68) | (SEQ ID NO: 16) |
| (mature SEQ ID NO: 66) | | | |
| 2-118-L3G4-BM | mutant β-NGF 118aa | GGGGGGSGGGG | modified IgG4 Fc |
| (prepro SEQ ID NO: 46) | (SEQ ID NO: 1) | SGGGGSA | (SEQ ID NO: 18) |
| (mature SEQ ID NO: 67) | | (SEQ ID NO: 69) | |

Nucleic acids encoding various preproNGF-Fc fusion proteins (e.g., “2-118-L3Fc10-M1-5”) or prepro-mNGF118 control were synthesized and cloned into pSC-T vectors (e.g., “pSC-2-118-L3Fc10-M1-5”) (by Shanghai Jierui Bio-Engineering Co., Ltd. Beijing Branch). PCR primers containing restriction sites HindIII and XhoI respectively were used to amplify the nucleic acids encoding various preproNGF-Fc fusion proteins or prepro-mNGF118 control, then PCR products were subcloned into in house generated eukaryotic expression vector pTT5 (e.g., “pTT5-2-118-L3Fc10-M1-5”).

### Expression of Recombinant Protein

293F cells transfected with eukaryotic expression vectors pTT5 carrying the nucleic acids encoding various preproNGF-Fc fusion proteins (e.g., pTT5-2-118-L3Fc10-M1-5) or control prepro-mNGF118 were cultured under 37° C., 8% CO₂, 120 rpm for 5 days. Supernatant was collected, which contained the expressed proteins.

### Purification of Recombinant Protein

Expressed NGF-Fc fusion proteins were first roughly purified by protein A affinity purification, then HiTrap™ Butyl HP column (GE Healthcare) was used to further separate them from host protein based on different hydrophobic properties. Residual aggregates were then removed with Superdex 200 Gel Filtration Column (GE Life Sciences) to arrive at purified mature NGF-Fc fusion proteins. Mature mNGF118 control was first purified with HiTrap™ Butyl HP column (GE Healthcare), then Superdex 200 Gel Filtration Column (GE Life Sciences). The purity of these proteins was verified by SDS-PAGE, which was above 90%.

### Example 2: Thermal Stability Studies of Mature NGF-Fc Fusion Proteins

Fluorescence protein analyzer UNcle (Unchained Labs) was used to measure the changes of fluorescence absorbance and light scattering under 266 nm/473 nm during sample heating, in order to calculate melting temperature (Tm) and onset aggregation temperature (Tagg) of samples, respectively. The initial temperature was set to 20° C., the end temperature was set to 95° C., the heating rate was 0.3° C./min. Each sample was repeated 3 times for measurements. Results are summarized in Table 2.

TABLE 2
Average melting temperature (Tm) and onset aggregation temperature (Tagg)
of mature NGF-Fc fusion proteins

| Construct (mature | Mature β-NGF | | | | | | |
|---|---|---|---|---|---|---|---|
| NGF-Fc) | moiety | Linker | Fc moiety | Tm | | Tagg | |
| FD-G4Fc | wt β-NGF 118aa | GSGGGSGGGGS | modified IgG4 Fc- | 62.4° | C. | 65° | C. |
| (mature SEQ ID NO: 55) | (SEQ ID NO: 3) | GGGGSGGGGS | FD | | | | |
| | | (SEQ ID NO: 71) | (SEQ ID NO: 19) | | | | |
| WM-G24Fc | wt β-NGF 118aa | KTGGGSGGGS | modified IgG2/4 Fc | 62° | C. | 63.7° | C. |
| (mature SEQ ID NO: 56) | (SEQ ID NO: 3) | (SEQ ID NO: 72) | (SEQ ID NO: 20) | | | | |
| NGF-1-15M7 | wt β-NGF 120aa | / | IgG1 M7 | 63.4° | C. | 71.4° | C. |
| (rhNGF-Fc1) | (SEQ ID NO: 4) | | (SEQ ID NO: 15) | | | | |
| (mature SEQ ID NO: 57) | | | | | | | |
| NGF-L3Fc10M7-5 | wt β-NGF 120aa | (G4S)₃ | IgG1 M7-5 (N′ | 63.7° | C. | 76.6° | C. |
| (rhNGF-Li-Fc1) | (SEQ ID NO: 4) | (SEQ ID NO: 68) | minus 5aa) | | | | |
| (mature SEQ ID NO: 58) | | | (SEQ ID NO: 16) | | | | |
| 2-1-15M7 | mutant β-NGF | / | IgG1 M7 | 63.3° | C. | 70° | C. |
| (rhNGF-(F12E)-Fc1) | 120aa | | (SEQ ID NO: 15) | | | | |
| (mature SEQ ID NO: 59) | (SEQ ID NO: 2) | | | | | | |
| NGF-4-12PAA | wt β-NGF 120aa | / | modified IgG4 Fc | 61.3° | C. | 62.5° | C. |
| (rhNGF-Fc4) | (SEQ ID NO: 4) | | (SEQ ID NO: 18) | | | | |
| (mature SEQ ID NO: 60) | | | | | | | |
| 2-118-L3Fc10-M1-5 | mutant β-NGF | (G4S)₃ | IgG1 M1-5 (N′ | 55.6° | C. | 54.7° | C. |
| (mature SEQ ID NO: 61) | 118aa | (SEQ ID NO: 68) | minus 5aa) | | | | |
| | (SEQ ID NO: 1) | | (SEQ ID NO: 10) | | | | |
| 2-118-L3Fc10-M3-5 | mutant β-NGF | (G4S)₃ | IgG1 M3-5 (N′ | 63.3° | C. | 77° | C. |
| (mature SEQ ID NO: 62) | 118aa | (SEQ ID NO: 68) | minus 5aa) | | | | |
| | (SEQ ID NO: 1) | | (SEQ ID NO: 12) | | | | |
| NGF-118-L3Fc10-M3-5 | wt β-NGF 118aa | (G4S)₃ | IgG1 M3-5 (N′ | 62.4° | C. | 76.2° | C. |
| (mature SEQ ID NO: 63) | (SEQ ID NO: 3) | (SEQ ID NO: 68) | minus 5aa) | | | | |
| | | | (SEQ ID NO: 12) | | | | |
| 2-L3Fc10-M3-5 | mutant β-NGF | (G4S)₃ | IgG1 M3-5 (N′ | 62.7° | C. | 75.6° | C. |
| (mature SEQ ID NO: 64) | 120aa | (SEQ ID NO: 68) | minus 5aa) | | | | |
| | (SEQ ID NO: 2) | | (SEQ ID NO: 12) | | | | |
| 2-118-L3Fc10-M5-5 | mutant β-NGF | (G4S)₃ | IgG1 M5-5 (N′ | 60.4° | C. | 77.9° | C. |
| (mature SEQ ID NO: 65) | 118aa | (SEQ ID NO: 68) | minus 5aa) | | | | |
| | (SEQ ID NO: 1) | | (SEQ ID NO: 14) | | | | |
| 2-118-L3Fc10M7-5 | mutant β-NGF | (G4S)₃ | IgG1 M7-5 (N′ | 64° | C. | 76° | C. |
| (mature SEQ ID NO: 66) | 118aa | (SEQ ID NO: 68) | minus 5aa) | | | | |
| | (SEQ ID NO: 1) | | (SEQ ID NO: 16) | | | | |
| 2-118-L3G4-BM | mutant β-NGF | GGGGGGSGGG | modified IgG4 Fc | 64.7° | C. | 66.5° | C. |

| | | | |
|---|---|---|---|
| (mature SEQ ID NO: 67) | 118aa | GSGGGGSA | (SEQ ID NO: 18) |
| | (SEQ ID NO: 1) | (SEQ ID NO: 69) | |

### Melting Temperature (Tm)

As can be seen from Table 2, 2-118-L3G4-BM showed the highest melting temperature (Tm), i.e., the best thermal stability, among all mature NGF-Fc fusion proteins comprising IgG4-derived Fc fragment (FD-G4Fc, WM-G24Fc, NGF-4-12PAA, and 2-118-L3G4-BM), or even among all mature NGF-Fc fusion proteins tested.

Among all mature NGF-Fc fusion proteins comprising IgG1-derived Fc fragment, 2-118-L3Fc10-M1-5 exhibited lower Tm (i.e., less thermal stability), while others showed similar Tm.

### Onset Aggregation Temperature (Tagg)

As can be seen from Table 2, mature NGF-Fc fusion proteins comprising IgG1-derived Fc fragment (except for 2-118-L3Fc10-M1-5) showed higher Tagg compared to that of mature NGF-Fc fusion proteins comprising IgG4-derived Fc fragment. This indicates that mature NGF-Fc fusion proteins comprising IgG1-derived Fc fragment are less prone to aggregate during heating process.

Among all mature NGF-Fc fusion proteins comprising IgG4-derived Fc fragment, 2-118-L3G4-BM exhibited the highest Tagg, demonstrating its superior anti-aggregation performance during heating process compared to other IgG4-derived Fc fusion constructs.

Among all mature NGF-Fc fusion proteins comprising IgG1-derived Fc fragment, 2-118-L3Fc10-M1-5 exhibited the lowest Tagg (worst anti-aggregation performance during heating process), NGF-1-15M7 and 2-1-15M7 showed relatively low Tagg, while the rest IgG1-derived Fc fusion constructs showed high and similar Tagg.

### Example 3: Accelerated Stability Testing of Mature NGF-Fc Fusion Proteins

Mature NGF-Fc fusion proteins were diluted with PBS to a final concentration of 2 mg/ml and incubated at 40° C. Samples were taken on Days 0 (“0h” in figures), 3, 7, 9, and 14 during the incubation period then stored at −80° C. The degradation and aggregation of the samples were detected by Size Exclusion Chromatography (SEC) and Capillary Electrophoresis-Sodium Dodecyl Sulfate (CE-SDS).

### Size Exclusion Chromatography (SEC) Detection Method

SEC separates molecules by differences in size as they pass through a resin packed in a column. Protein samples were centrifuged at 10,000 g for 5 minutes at 4° C. The pellets were resuspended with PBS. 80-100 μL samples were transferred to a 384-well plate for detection on Waters® ACQUITY UPLC® H-Class Bio Tunable UV (TUV) Detector, with injection volume of 20 μL, wavelength 280 nm, flow speed 0.25 mL/min, and total running time of 17 minutes. Mobile phase buffer contained 100 mM PB (80 mM Na₂HPO₄, 20 mM NaH₂PO₄), 300 mM NaCl, 10% acetonitrile, pH 7.2.

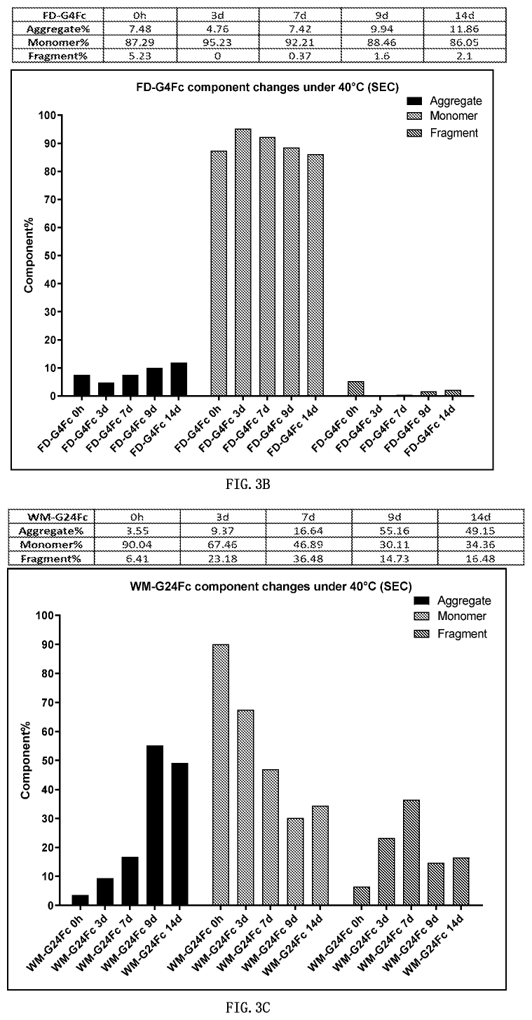

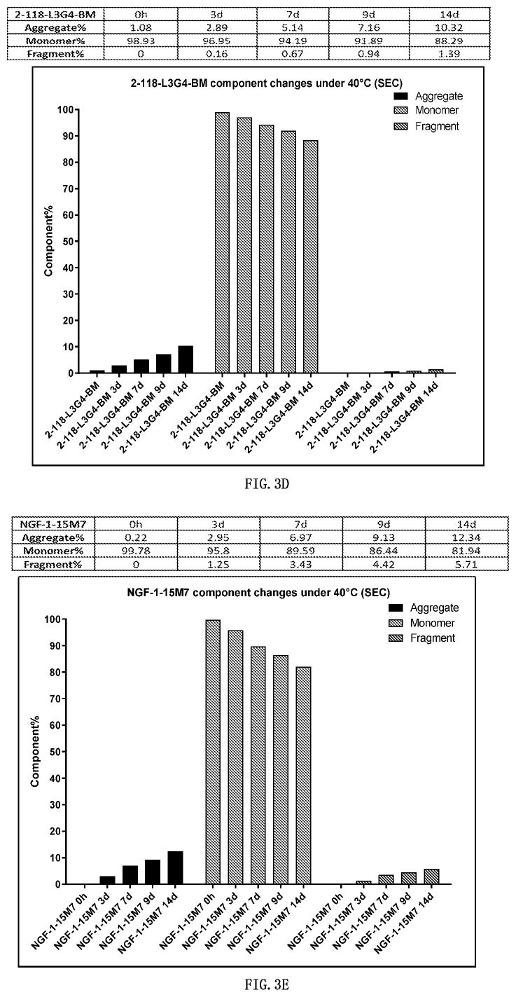

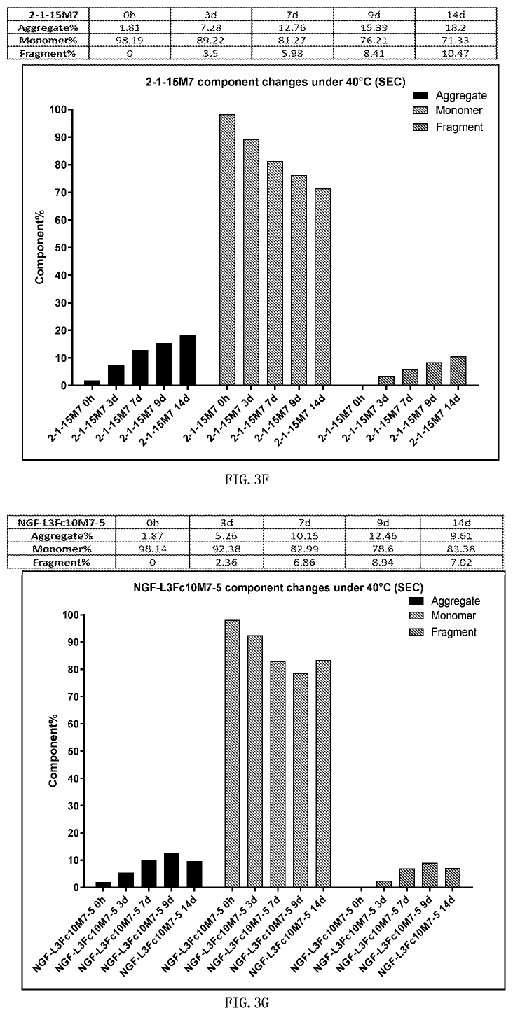

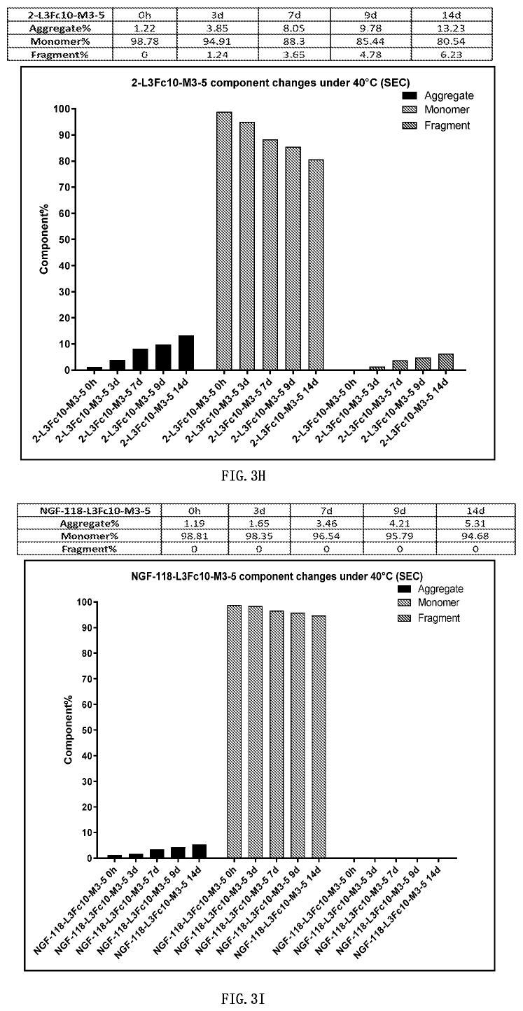

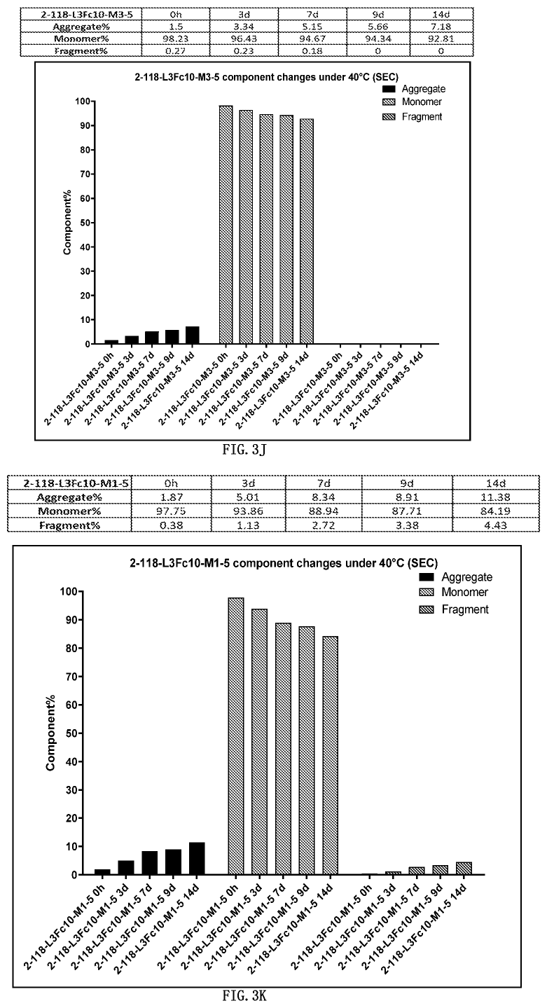

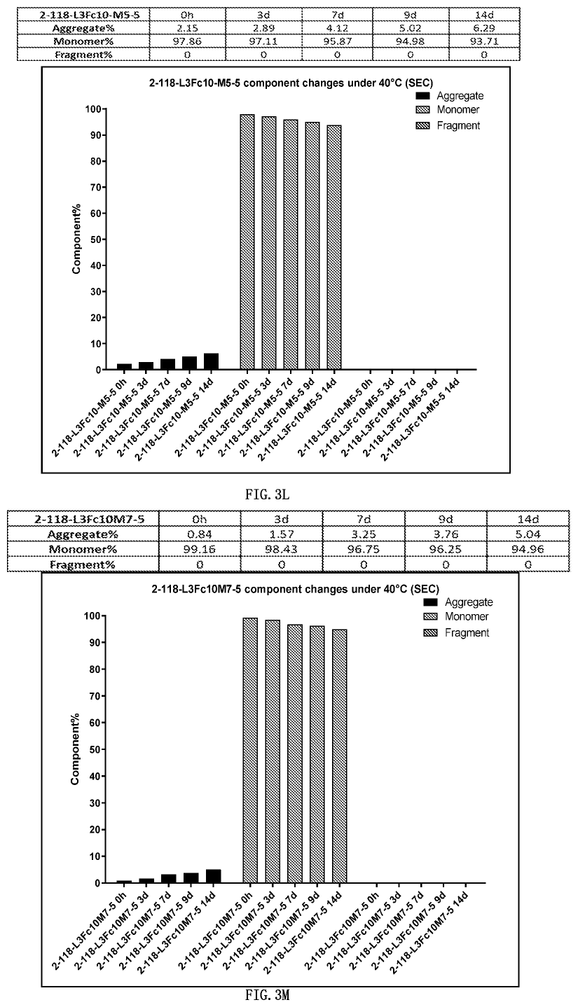

As shown in Table 3 and FIGS. 3A-3D, among all mature NGF-Fc fusion proteins comprising IgG4-derived Fc fragment, 2-118-L3G4-BM and FD-G4Fc exhibited superior stability over WM-G24Fc and NGF-4-12PAA in terms of aggregate increase and fragment generation. NGF-4-12PAA showed very obvious fragmentation and the percentage of fragmentation reached 49.59% (FIG. 3A), while the percentages of fragmentation of 2-118-L3G4-BM and FD-G4Fc were below 2.1% during accelerated stress. WM-G24Fc had not only significant aggregate increase (percentage of aggregate reached 49.15%), but also obvious fragmentation during accelerated stress (FIG. 3C). The monomer percentages of 2-118-L3G4-BM and FD-G4Fc were above 85%, while the monomer percentages of WM-G24Fc and NGF-4-12PAA decreased obviously with time. Compared to 2-118-L3G4-BM, FD-G4Fc showed more aggregate formation and fragmentation during accelerated stress, suggesting 2-118-L3G4-BM has better anti-fragmentation and anti-aggregatation activity compared to FD-G4Fc.

As shown in Table 3 and FIGS. 3E-3M, among all mature NGF-Fc fusion proteins comprising IgG1-derived Fc fragment, 2-118-L3Fc10-M3-5, 2-118-L3Fc10-M5-5, 2-118-L3Fc10M7-5, and NGF-118-L3Fc10-M3-5 exhibited significantly better stability under accelerated stress than other IgG1-derived Fc fusion proteins, with no detectable fragment formation (0%) and much less aggregate increment. 2-118-L3Fc10-M1-5 also showed comparable or better stability than IgG1-derived Fc fusion proteins such as NGF-1-15M7, NGF-L3Fc10M7-5, and 2-1-15M7.

TABLE 3
SEC measurement of various component % and aggregate increment % of
mature NGF-Fc fusion proteins under 40° C. accelerated stability
test (Day 14 sample)

| Construct (mature | Mature β- | | | aggregate | fragment | Main | Aggregate |
|---|---|---|---|---|---|---|---|
| NGF-Fc) | NGF moiety | Linker | Fc moiety | % | % | peak % | increase % |
| FD-G4Fc | wt β-NGF | GSGGGSG | modified IgG4 | 11.86 | 2.1 | 86.05 | 4.38 |
| | 118aa | GGGSGGG | Fc-FD | | | | |
| | | GSGGGGS | | | | | |
| WM-G24Fc | wt β-NGF | KTGGGSG | modified | 49.15 | 16.48 | 34.36 | 45.6 |
| | 118aa | GGS | IgG2/4 Fc | | | | |
| NGF-1-15M7 | wt β-NGF | / | IgG1 M7 | 12.34 | 5.71 | 81.94 | 12.12 |
| (rhNGF-Fc1) | 120aa | | | | | | |
| NGF-L3Fc10M7-5 | wt β-NGF | (G4S)₃ | IgG1 M7-5 | 9.61 | 7.02 | 83.38 | 7.74 |
| (rhNGF-Li-Fc1) | 120aa | | (N′ minus 5aa) | | | | |
| 2-1-15M7 | mutant β- | / | IgG1 M7 | 18.2 | 10.47 | 71.33 | 16.39 |
| (rhNGF-(F12E)- | NGF 120aa | | | | | | |
| Fc1) | | | | | | | |
| NGF-4-12PAA | wt β-NGF | / | modified IgG4 | 9.45 | 49.59 | 40.96 | 7.07 |
| (rhNGF-Fc4) | 120aa | | Fc | | | | |
| 2-118-L3Fc10-M1-5 | mutant β- | (G4S)₃ | IgG1 M1-5 | 11.38 | 4.43 | 84.19 | 9.51 |
| | NGF 118aa | | (N′ minus 5aa) | | | | |
| 2-118-L3Fc10-M3-5 | mutant β- | (G4S)₃ | IgG1 M3-5 | 7.18 | 0 | 92.81 | 5.68 |
| | NGF 118aa | | (N′ minus 5aa) | | | | |
| NGF-118-L3Fc10- | wt β-NGF | (G4S)₃ | IgG1 M3-5 | 5.31 | 0 | 94.68 | 4.12 |
| M3-5 | 118aa | | (N′ minus 5aa) | | | | |
| 2-L3Fc10-M3-5 | mutant β- | (G4S)₃ | IgG1 M3-5 | 13.23 | 6.23 | 80.54 | 12.01 |
| | NGF 120aa | | (N′ minus 5aa) | | | | |
| 2-118-L3Fc10-M5-5 | mutant β- | (G4S)₃ | IgG1 M5-5 | 6.29 | 0 | 93.71 | 4.14 |
| | NGF 118aa | | (N′ minus 5aa) | | | | |
| 2-118-L3Fc10M7-5 | mutant β- | (G4S)₃ | IgG1 M7-5 | 5.04 | 0 | 94.96 | 4.2 |
| | NGF 118aa | | (N′ minus 5aa) | | | | |
| 2-118-L3G4-BM | mutant β- | GGGGGGS | modified IgG4 | 10.32 | 1.39 | 88.29 | 9.24 |
| | NGF 118aa | GGGGSGG | Fc | | | | |
| | | GGSA | | | | | |

### Capillary Electrophoresis-Sodium Dodecyl Sulfate (CE-SDS) Detection Method

In capillary electrophoresis, samples are separated in the capillary based on electrophoretic mobility, which varies with charge and size of the molecules. First, 40 μL 1× Sample Buffer and 10 μL protein sample were mixed in a centrifuge tube to obtain a 50 μL mixture containing final concentration of 0.4 μg/μL protein. 1 μL reconstituted 25× Internal Standard was added into the mixture, followed by adding in 2.5 μL 250 mM Iodoacetamide. The whole mixture was vortexed and incubated at 70° C. for 10 min, then let cool down, followed by well mixing and then centrifugation. The supernatant of 50 μL of the processed sample was transfered into a 96-well plate. The 96-well plate was centrifuged at 1,000 g for 10 min, and was then placed into the Maurice system (ProteinSimple) for CE-SDS detection according to standard protocol.

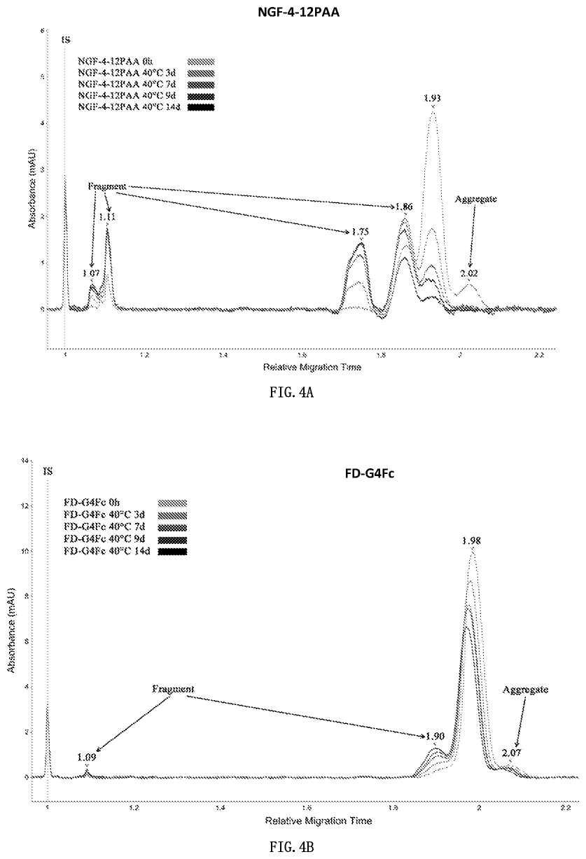

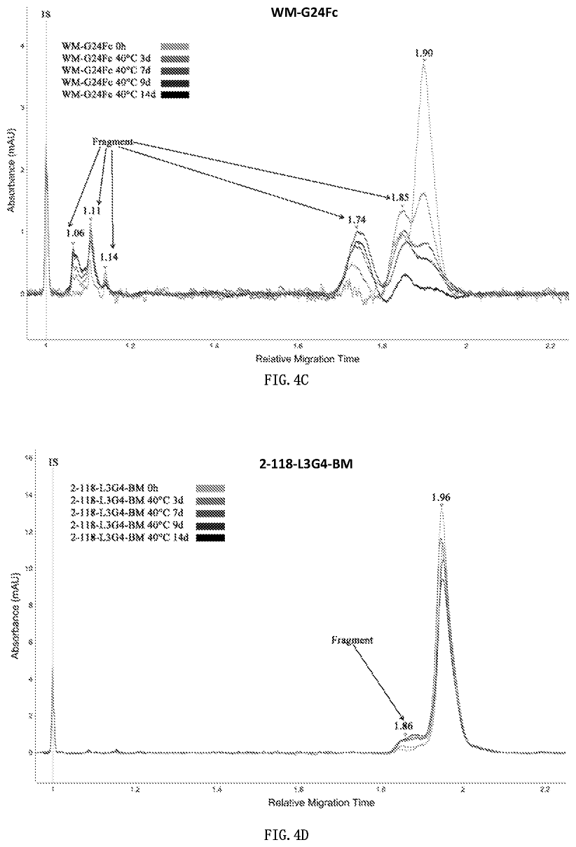

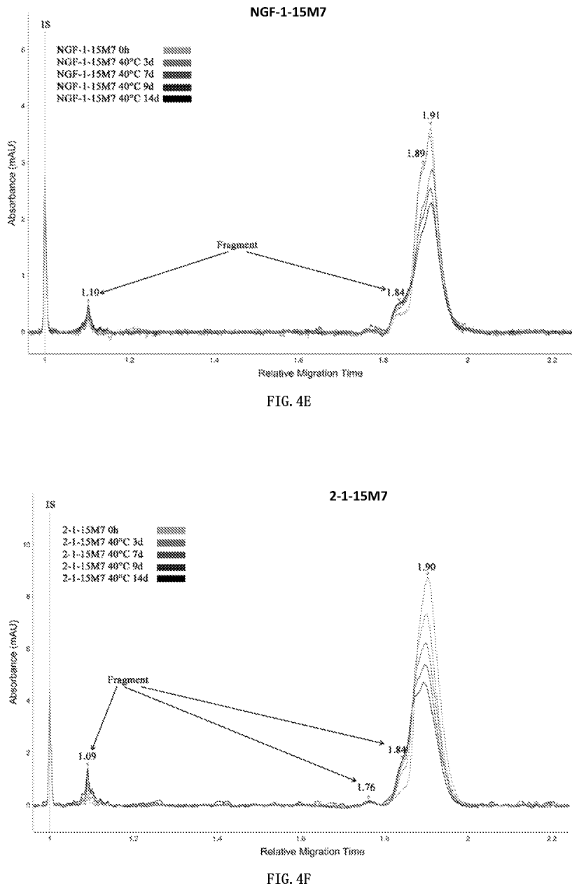

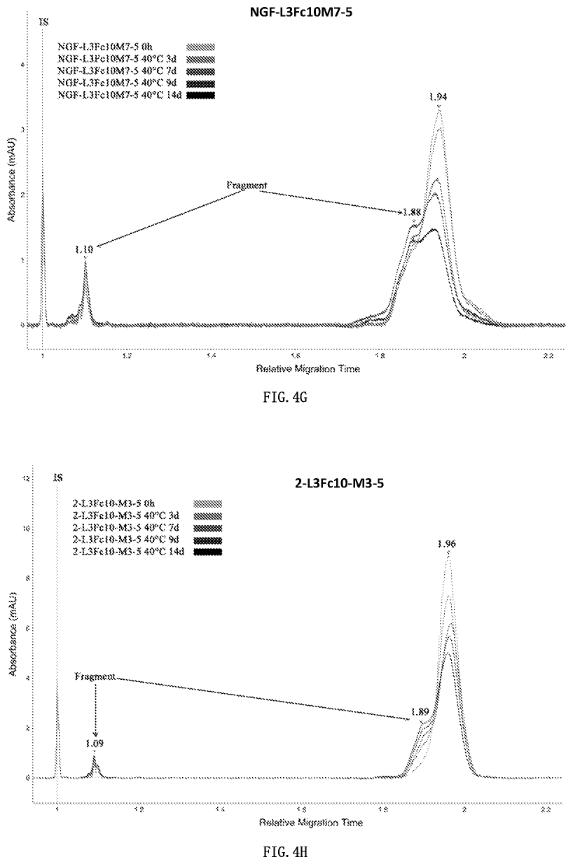

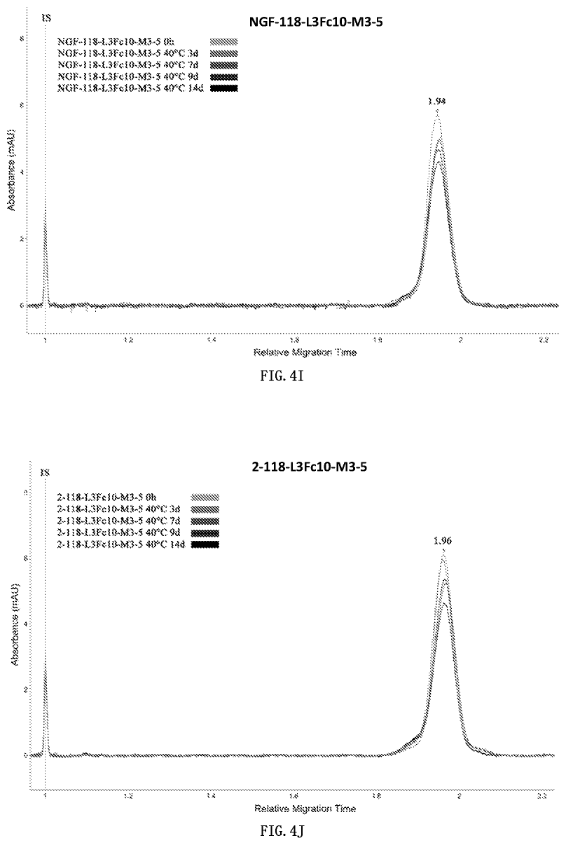

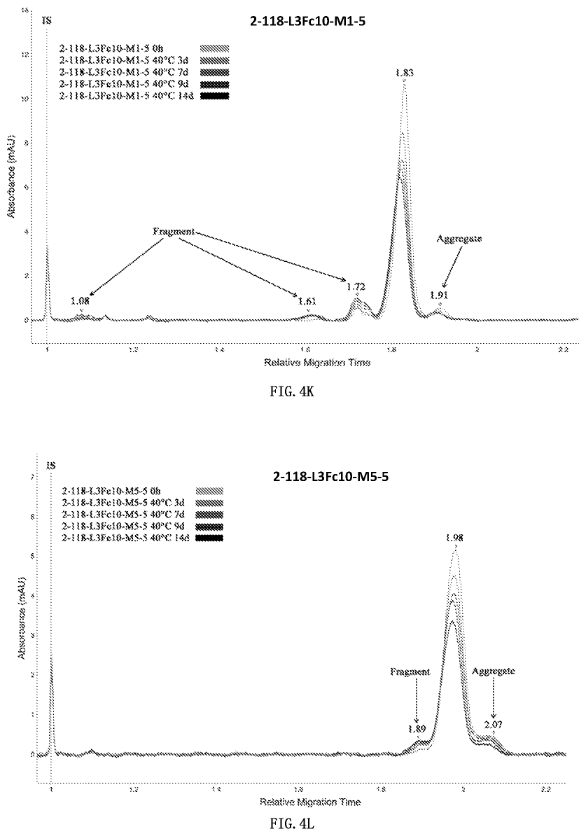

As shown in Table 4 and FIGS. 4A-4D, among all mature NGF-Fc fusion proteins comprising IgG4-derived Fc fragment, during accelerated stress, NGF-4-12PAA and WM-G24Fc showed very obvious fragmentation; and 2-118-L3G4-BM had the lowest fragmentation. This is consistent with the SEC results. Aggregate peaks showed up on the right side of the main peaks for FD-G4Fc (FIG. 4B), but this was not seen for 2-118-L3G4-BM (FIG. 4D). In conclusion, 2-118-L3G4-BM has much better stability during accelerated stress compared to other IgG4-derived Fc fusion proteins, which is consistent with the SEC results.

As can be seen from Table 4 and FIGS. 4E-4M, among all mature NGF-Fc fusion proteins comprising IgG1-derived Fc fragment, 2-118-L3Fc10M7-5, NGF-118-L3Fc10-M3-5, and 2-118-L3Fc10-M3-5 had the best stability during accelerated stress, with no obvious fragment peaks showing up (see FIGS. 41, 4J, and 4M), and fragment increment of Day 14 sample was 0%. 2-118-L3Fc10-M5-5 also had good stability with much less fragment increment (5.8% for Day 14). In contrast, other NGF-Fc fusion proteins comprising IgG1-derived Fc fragment (e.g., NGF-L3Fc10M7-5) were more prone to fragment formation.

TABLE 4
CE-SDS measurement of fragment % and fragment increment % of mature
NGF-Fc fusion proteins under 40° C. accelerated stability test
(Day 14 sample)

| Construct (mature | Mature β-NGF | | | Fragment | Fragment |
|---|---|---|---|---|---|
| NGF-Fc) | moiety | Linker | Fc moiety | % | increase % |
| FD-G4Fc | wt β-NGF 118aa | GSGGGSGGG | modified IgG4 | 17.9 | 14.4 |
| | | GSGGGGSGG | Fc-FD | | |
| | | GGS | | | |
| WM-G24Fc | wt β-NGF 118aa | KTGGGSGGG | modified | 91.6 | 67.8 |
| | | S | IgG2/4 Fc | | |
| NGF-1-15M7 | wt β-NGF 120aa | / | IgG1 M7 | 15.8 | 8.6 |
| (rhNGF-Fc1) | | | | | |
| NGF-L3Fc10M7-5 | wt β-NGF 120aa | (G4S)₃ | IgG1 M7-5 | 47 | 31 |
| (rhNGF-Li-Fc1) | | | (N′ minus 5aa) | | |
| 2-1-15M7 | mutant β-NGF 120aa | / | IgG1 M7 | 27 | 22.6 |
| (rhNGF-(F12E)-Fc1) | | | | | |
| NGF-4-12PAA | wt β-NGF 120aa | / | modified IgG4 | 91.4 | 65.4 |
| (rhNGF-Fc4) | | | Fc | | |
| 2-118-L3Fc10-M1-5 | mutant β-NGF 118aa | (G4S)₃ | IgG1 M1-5 | 16.7 | 11.9 |
| | | | (N′ minus 5aa) | | |
| 2-118-L3Fc10-M3-5 | mutant β-NGF 118aa | (G4S)₃ | IgG1 M3-5 | 0 | 0 |
| | | | (N′ minus 5aa) | | |
| NGF-118-L3Fc10-M3-5 | wt β-NGF 118aa | (G4S)₃ | IgG1 M3-5 | 0 | 0 |
| | | | (N′ minus 5aa) | | |
| 2-L3Fc10-M3-5 | mutant β-NGF 120aa | (G4S)₃ | IgG1 M3-5 | 27.4 | 27.4 |
| | | | (N′ minus 5aa) | | |
| 2-118-L3Fc10-M5-5 | mutant β-NGF 118aa | (G4S)₃ | IgG1 M5-5 | 5.8 | 5.8 |
| | | | (N′ minus 5aa) | | |
| 2-118-L3Fc10M7-5 | mutant β-NGF 118aa | (G4S)₃ | IgG1 M7-5 | 0 | 0 |
| | | | (N′ minus 5aa) | | |
| 2-118-L3G4-BM | mutant β-NGF 118aa | GGGGGGSGG | modified IgG4 | 11.4 | 10 |
| | | GGSGGGGSA | Fc | | |

To summarize, based on SEC and CE-SDS results: 1) among all mature NGF-Fc fusion proteins comprising IgG4-derived Fc fragment, 2-118-L3G4-BM showed better accelerated stability than FD-G4Fc, and significantly superior accelerated stability than WM-G24Fc and NGF-4-12PAA; 2) among all mature NGF-Fc fusion proteins comprising IgG1-derived Fc fragment, 2-118-L3Fc10-M3-5, NGF-118-L3Fc10-M3-5, and 2-118-L3Fc10M7-5 showed the best accelerated stability, followed by 2-118-L3Fc10-M5-5, compared to all other constructs; and 3) relatively speaking, mature NGF-Fc fusion proteins comprising IgG1-derived Fc fragment showed better anti-aggregation property during accelerated stress compared to those comprising IgG4-derived Fc fragment.

### Example 4: TF-1 Cell Proliferation Assay for Estimating Bioactivity of NGF-Fc Fusion Proteins

TF-1 cell proliferation assay was used to test the bioactivities of different NGF-Fc fusion proteins.

TF-1 cells are a factor-dependent human erythroleukemic cell line. TF-1 cells were resuspended in the basic culture medium (RPMI 1640 medium+10% FBS) to arrive at a suspension containing 5.0×10⁴ cells/mL for later use. A standard solution was prepared for SuTaiSheng® mouse NGF (standard control), test solutions of various NGF-Fc fusion proteins and control solutions of mNGF118 (mutant β-NGF 118aa) and rhNGF (recombinant human wildtype β-NGF 120aa, SEQ ID NO: 4, prepared and purified as described in Example 1) were prepared, so that the final protein was 200U/mL×100 μL/well in a pre-labeled 96-well plate. Then, 100 μL of 5.0×10⁴ cells/mL TF-1 cell suspension was added to each well of the 96-well plate containing standard control (SuTaiSheng® mouse NGF), test NGF-Fc fusion protein, or control solution, then incubated under 37° C., 5% CO₂ for 72 hours in a humid incubator. 20 μL assay solution from CellTiter 96® AQueous One Solution Cell Proliferation Assay (Promega, Cat #G3581) was added into each well of the cell suspension, then incubated under 37° C., 5% CO₂ for 3 hours. The plate was then measured for absorbance under 490 nm and 650 nm in a spectrophotometer. Data were recorded and normalized to the standard NGF control (SuTaiSheng® mouse NGF).

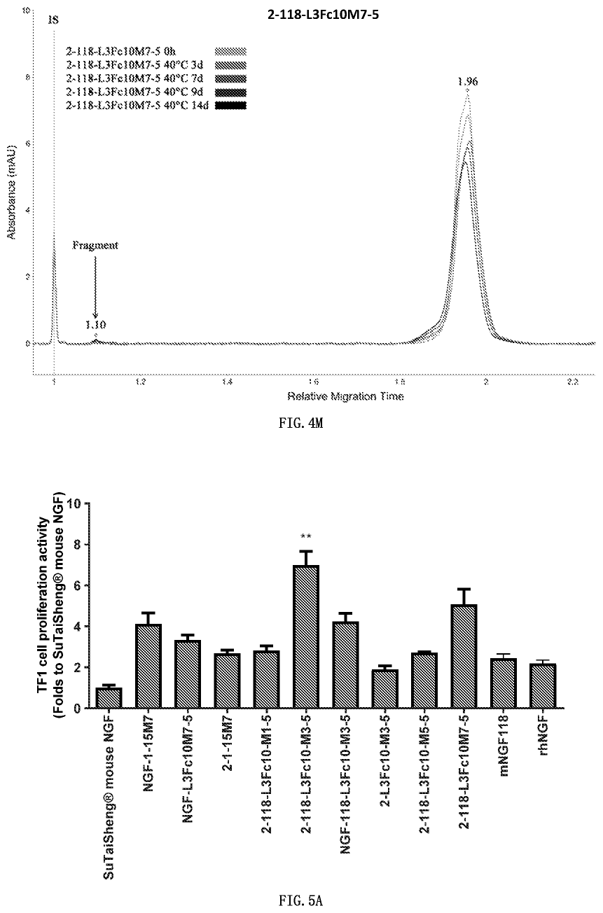

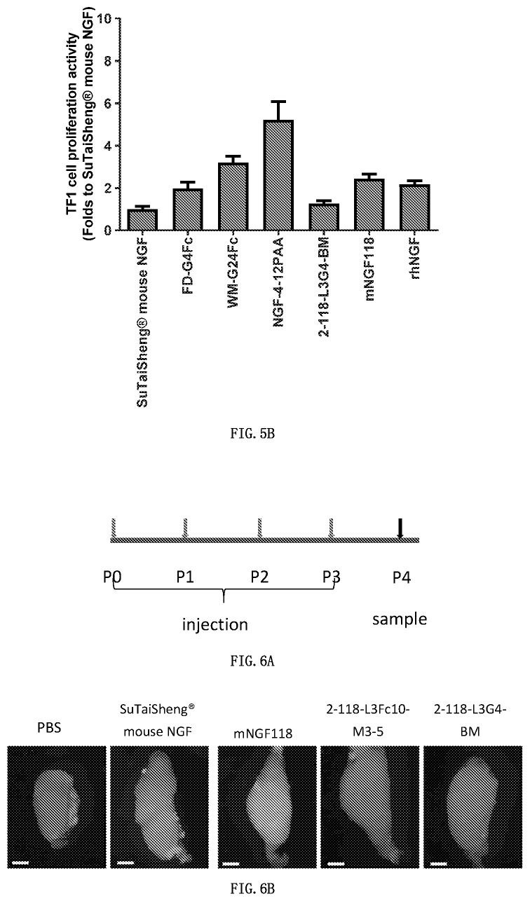

As can be seen from FIG. 5A, 2-118-L3Fc10-M3-5 demonstrated the highest bioactivity in promoting TF-1 cell proliferation among all mature NGF-Fc fusion proteins comprising IgG1-derived Fc fragment (or even among all NGF constructs including rhNGF, mNGF118, and SuTaiSheng® mouse NGF); 2-118-L3Fc10-M3-5 also had the highest bioactivity among the three most stable constructs (2-118-L3Fc10-M3-5, NGF-118-L3Fc10-M3-5, and 2-118-L3Fc10M7-5) under accelerated stability assay (see Example 3). As can be seen from FIG. 5B, all mature NGF-Fc fusion proteins comprising IgG4-derived Fc fragment exhibited bioactivity in promoting TF-1 cell proliferation, and 2-118-L3G4-BM demonstrated comparable bioactivity to standard control SuTaiSheng® mouse NGF.

### Example 5: In Vivo Bioactivity Assay for Various NGF-Fc Fusion Proteins in Rats

Superior cervical ganglion (SCG) is a tissue composed of approximately 30,000 neurons, and is one of the most sensitive tissues to NGF, especially during prenatal and postnatal development. In the TF-1 cell proliferation assay (see Example 4), certain NGF-Fc fusion proteins comprising IgG1- or IgG4-derived Fc fragment demonstrated great bioactivity. Various NGF-Fc fusion proteins were injected into rat SCG, and SCG size was measured at different timepoints post-injection, in order to assess the activity of NGF-Fc fusion proteins in promoting SCG growth in vivo.

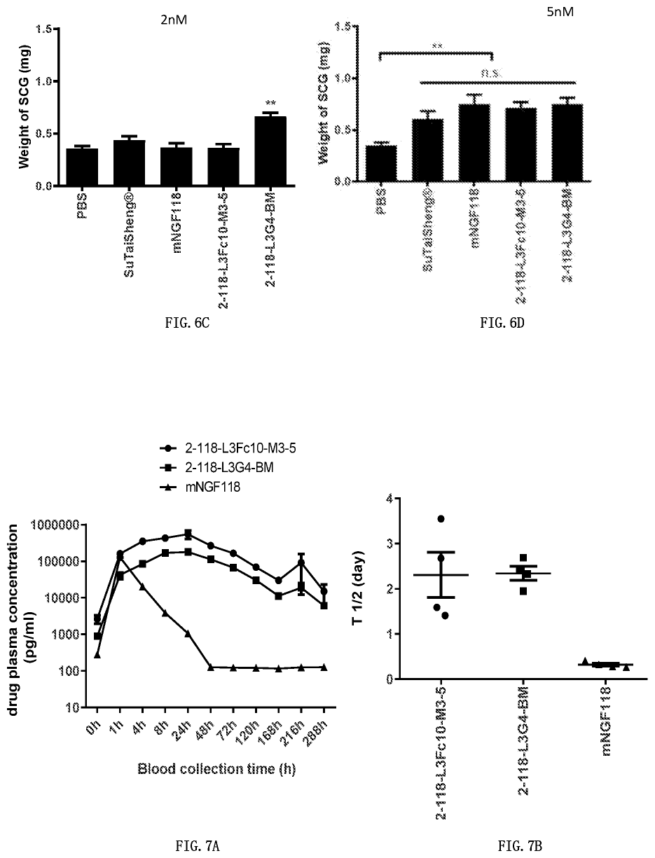

Newborn Sprague Dawley (SD) rats were subcutaneously administered to the neck with various NGF-Fc fusion proteins or NGF controls (SuTaiSheng® mouse NGF, or mutant NGF118), then sacrificed for SCG dissection. PBS injection served as negative control. As shown in FIG. 6A, NGF control protein (SuTaiSheng® mouse NGF or mutant NGF118) or PBS was injected on Day 0, 1, 2, and 3 once per day, then SCGs were obtained on Day 4. 2-118-L3Fc10-M3-5 or 2-118-L3G4-BM was injected on Day 0 with single injection at the same dose, then SCGs were obtained on Day 4. Briefly, after decapitation, rat head was fixed on an operating platform, blood was drained with cotton balls, trachea and foramen magnum were first located, then the carotid sheath tissue on the obliquely posterior side of the trachea was located and removed with a micro tweezer and placed into a petri dish containing PBS, followed by SCG isolation under a dissecting microscope. Excess liquid on the surface of the isolated SCG was removed with paper tissue, then SCG was placed onto a clean surface dish for weight measurement. The morphology of the SCGs is demonstrated in FIG. 6B. Recorded data were analyzed with Student t test. ** indicates statistically significant compared to the PBS treated group; n.s. indicates “no significance” compared to the PBS treated group. As can be seen from FIG. 6C, at the administration dosage of 2 nM, mutant β-NGF 118aa (mNGF118), SuTaiSheng® mouse NGF, and 2-118-L3Fc10-M3-5 did not promote SCG growth with statistical significance compared to PBS negative control. While 2-118-L3G4-BM significantly promoted SCG growth compared to PBS control (** p<0.01). At the administration dosage of 5 nM, either NGF controls (SuTaiSheng® mouse NGF or mNGF118) or NGF-Fc fusion proteins (2-118-L3G4-BM or 2-118-L3Fc10-M3-5) promoted in vivo SCG growth significantly when compared to the PBS treated group (** p<0.01), and the activities of promoting SCG growth were not significantly different among the four tested NGF proteins (n.s. indicates p>0.05) (FIG. 6D). Thus, single subcutaneous injection of 2-118-L3G4-BM demonstrated superior activity in promoting in vivo SCG growth compared to SuTaiSheng® mouse NGF or mutant β-NGF 118aa (mNGF118) even at the dose of 2 nM. While at higher dosage (5 nM), single treatment of either 2-118-L3G4-BM or 2-118-L3Fc10-M3-5 demonstrated similar activity in promoting SCG growth.

### Example 6: Pharmacokinetic (PK) Studies of Various NGF Polypeptides in Rats

We tested PK profiles of various NGF constructs by injecting into adult rats.

24 male SD rats (6-8 weeks old, about 250 g-300 g per rat) were randomly divided into 3 groups (8 in each group), and intramuscularly injected 235 μg/kg each of 2-118-L3Fc10-M3-5, 2-118-L3G4-BM, or mNGF118 (mutant β-NGF 118aa without Fc fusion), respectively. 150 μL blood was collected from posterior orbital vein before-injection (0 hr), or at 1 hr, 4 hrs, 8 hrs, 24 hrs, 48 hrs, 72 hrs, 120 hrs, 168 hrs, 216 hrs, and 288 hrs post-injection. Plasma was isolated after blood collection, then used for NGF content testing with Human NGF Matched ELISA Antibody Pair Set (Sino Biological, SEK11050). The average ODs of the standards under 450 nm-630 nm were plotted against the Y-axis, and the concentrations of the standards were plotted against the X-axis, to generate standard curve linear equation, with the requirement of R²>0.98. Then blood plasma concentrations of different samples were calculated based on the standard curve linear equation. GraphPad Prism 5.0 was used make semi-log plot of sample concentration vs. time, Phoenix WinNonlin 6.2 was used for PK analysis, and GraphPad Prism 5.0 was used to plot half-life scatter plots.

As can be seen from FIG. 7A, NGF-Fc fusion proteins showed much higher blood plasma concentration over time after a single intramuscular injection compared to mNGF118 control without Fc fusion; and 2-118-L3G4-BM showed similar blood plasma concentration over time after a single intramuscular injection compared to 2-118-L3Fc10-M3-5. As can be seen from FIG. 7B, the half-life of 2-118-L3G4-BM (55 hrs) was almost the same to that of 2-118-L3Fc10-M3-5 (55 hrs), both were much longer than (about 31 folds) mNGF118 control without Fc fusion (half-life 1.75 hrs). These results further explained why a single dose of 2-118-L3G4-BM or 2-118-L3Fc10-M3-5 showed similar activity in promoting in vivo SGC growth as compared to continuous injection of non-Fc fusion NGF controls (SuTaiSheng® mouse NGF or mNGF118) (see FIG. 6D).

Half-lives of wtNGF120 (i.e., “rhNGF”, human wildtype β-NGF 120aa, SEQ ID NO: 4), NGF-1-15M7 (rhNGF-Fc1), NGF-L3Fc10M7-5 (rhNGF-Li-Fc1), 2-1-15M7 (rhNGF-(F12E)-Fc1), and NGF-4-12PAA (rhNGF-Fc4) were described in Table 2 of WO2017157325 and summarized in Table 5. The half-life of mNGF118 tested in this experiment (1.75 hrs) was similar to that of wtNGF120 tested in WO2017157325 (1.8 hrs). As can be seen from Table 5, NGF-1-15M7 (rhNGF-Fc1), NGF-L3Fc10M7-5 (rhNGF-Li-Fc1), and 2-1-15M7 (rhNGF-(F12E)-Fc1) constructs extended in vivo half-life for more than 17 folds compared to wtNGF120 or mNGF118 controls without Fc fusion; also about 1.4 folds more than that of NGF-4-12PAA (rhNGF-Fc4). The half-life of 2-118-L3G4-BM (55 hrs) tested here was almost the same to that of 2-118-L3Fc10-M3-5 (55 hrs), both about 31 folds compared to wtNGF120 or mNGF118 controls without Fc fusion, and also much longer than all previously tested mature NGF-Fc fusion proteins with either IgG1- or IgG4-derived Fc fragment.

TABLE 5
Half-life of mature NGF polypeptides

| | Mature β-NGF | | | Half-life |
|---|---|---|---|---|
| Construct | moiety | Linker | Fc moiety | (t1/2, hrs) |
| wtNGF120 | wt β-NGF 120aa | / | / | 1.8 |
| (rhNGF) | | | | |
| NGF-1-15M7 | wt β-NGF 120aa | / | IgG1 M7 | 38.75 |
| (rhNGF-Fc1) | | | | |
| NGF-L3Fc10M7-5 | wt β-NGF 120aa | (G4S)₃ | IgG1 M7-5 (N′ | 33.83 |
| (rhNGF-Li-Fc1) | | | minus 5aa) | |
| 2-1-15M7 | mutant β-NGF 120aa | / | IgG1 M7 | 32.24 |
| (rhNGF-(F12E)-Fc1) | | | | |
| NGF-4-12PAA | wt β-NGF 120aa | / | modified IgG4 Fc | 23.1 |
| (rhNGF-Fc4) | | | | |
| mNGF118 | mutant β-NGF 118aa | / | / | 1.75 |
| 2-118-L3Fc10-M3-5 | mutant β-NGF 118aa | (G4S)₃ | IgG1 M3-5 (N′ | 55 |
| | | | minus 5aa) | |
| 2-118-L3G4-BM | mutant β-NGF 118aa | GGGGGGSGG | modified IgG4 Fc | 55 |
| | | GGSGGGGSA | | |

### Example 7: NGF-Fc Fusion Protein Promotes Wound Healing of Diabetic Neuropathy

Diabetic neuropathy is one of the common chronic complications of diabetes, the patients of which are characterized by slow wound healing, different degrees of infection, ulcers, and anthrax, even with a risk of amputation. This Example illustrates the study of therapeutic effects of NGF-Fc fusion proteins in a diabetic neuropathy animal model (e.g., through evaluation of wound healing).

CD-1 mice were obtained from Beijing Vital River Laboratory Animal Technology Co., Ltd. Animal model of diabetes was established with standard methods (e.g., see G. Graiani et al., “Nerve growth factor promotes reparative angiogenesis and inhibits endothelial apoptosis in cutaneous wounds of Type 1 diabetic mice.” Diabetologia. 2004, 47 (6): 1047-54). After 4 weeks of induction of diabetes, mice were anesthetized, and two full-thickness skin wounds of 4 mm in diameter were generated side by side in interscapular region by a disposable skin punch equipment. 50 μg/mL SuTaiSheng® mouse NGF, mNGF118, or NGF-Fc fusion protein (2-118-L3Fc10-M3-5 or 2-118-L3G4-BM) was administered to the right wound with a dose of 20 μL/administration. PBS of equal volume was administered to the left wound (serving as negative control). PBS, SuTaiSheng® mouse NGF, or mNGF118 was administered on Days 0 (after drilling mice on the back), 1, 2, and 3, once per day. 2-118-L3Fc10-M3-5 or 2-118-L3G4-BM of the same dose was administrated on Day 0 (after drilling) with single administration. The wound area was measured immediately after drilling and recorded as wound area of Day 0. Then wound areas on Days 4 and 7 were measured followed by calculating wound closure rate. Recorded data were analyzed by Student t test. Histogram was drawn by GraphPad Prism 8.0.1.

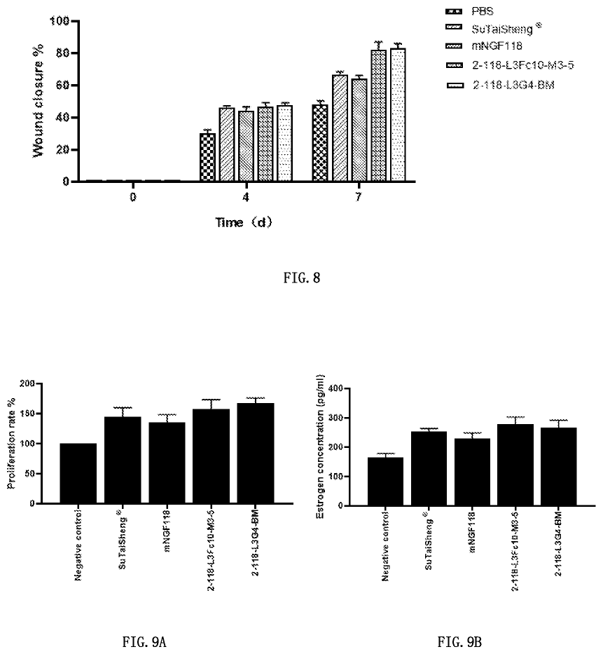

As shown in FIG. 8, would closure on Day 7 was improved compared to Day 4 for all groups; SuTaiSheng® mouse NGF, mNGF118, and NGF-Fc fusion protein (2-118-L3Fc10-M3-5 or 2-118-L3G4-BM) all promoted diabetic wound healing significantly, when compared to PBS negative control (p<0.01). Specifically, on Day 4, the average wound area in PBS treatment group was about 1.3 times bigger than that in SuTaiSheng® mouse NGF, mNGF118, or NGF-Fc fusion protein treatment group. These results demonstrate that SuTaiSheng® mouse NGF, mNGF118, or NGF-Fc fusion protein can effectively improve the slow wound healing defect in diabetic mice. Further, on Day 7, wound closure rate in diabetic mice administrated with NGF-Fc fusion protein was higher than that in both SuTaiSheng® mouse NGF and mNGF118 treatment group. This demonstrated superior in vivo therapeutic effect of NGF-Fc fusion protein over NGF (e.g., SuTaiSheng® mouse NGF, or mNGF118).

### Example 8: Therapeutic Effect of NGF-Fc Fusion Protein on Alzheimer's Disease

Alzheimer's disease (AD) is a degenerative disease of central nervous system with progressive memory loss as main clinical manifestation, which mostly occurs in the elderly with complex pathogenesis. This Example illustrates the study of therapeutic effects of NGF-Fc fusion proteins on AD in vivo (e.g., through evaluation of animal behavioral changes).

Wistar rats were used to establish animal model of AD with standard methods (for example, see G. L. Wenk et al. “Basal forebrain neurons and memory: a biochemical, histological and behavioural study of differential vulnerability to ibotenate and quisqualate.” Behav Neurosci, 1992, 106 (6): 909-923). Briefly, Wistar rats received stereotactic injection of ibotenic acid (IBO). Two days after IBO administration, anesthetized AD model rats were placed on their backs. 150 μg/mL NGF (SuTaiSheng® mouse NGF or mNGF118) or NGF-Fc fusion protein (2-118-L3Fc10-M3-5 or 2-118-L3G4-BM) was administrated intranasally at a total dose of 100 μL/administration. AD model rats administrated with PBS of equal volume served as negative control group. NGF or PBS was administrated once per day for 7 days continuously. NGF-Fc fusion protein was administrated on Day 1 with a single administration. The behavioral changes of rats were evaluated by Morris water maze (MWM) on Day 7. Briefly, using a Morris water maze device, rats were trained to climb the platform before experiment. The platform seeking time (escape latency; from entering the water to climbing onto the platform), and the times of crossing the position where the platform had been placed but then removed within 120s, were recorded during the experiment. Recorded data were analyzed by Student t test.

As shown in Table 6, the platform seeking time of AD model rats treated by SuTaiSheng® mouse NGF, mNGF118, or NGF-Fc fusion protein (2-118-L3Fc10-M3-5 or 2-118-L3G4-BM) was significantly shortened (*p<0.05), and the platform crossing times were significantly increased (*p<0.05), compared to the negative control group. Thus, NGFs and NGF-Fc fusion proteins described herein can both effectively improve spatial cognition, memory, and learning ability ability of AD model rats. Further, AD model rats treated by either 2-118-L3Fc10-M3-5 or 2-118-L3G4-BM seem to have a shorter platform seeking time and higher occurrence of platform crossing compared to rats treated by NGF (SuTaiSheng® mouse NGF or mNGF118). These data demonstrate superior in vivo therapeutic effect of NGF-Fc fusion protein on AD.

TABLE 6
Statistical table of Morris water maze (x̅ ± std)

| | Platform seeking | Platform crossing |
|---|---|---|
| Group | time (s) | times (occurrence) |
| Negative control PBS | 52.28 ± 20.12 | 3.31 ± 1.85 |
| SuTaiSheng ® mouse NGF | 32.05 ± 16.36* | 7.43 ± 2.85* |
| mNGF118 | 31.91 ± 18.87* | 7.58 ± 3.26* |
| 2-118-L3Fc10-M3-5 | 28.16 ± 20.85* | 8.01 ± 3.07* |
| 2-118-L3G4-BM | 27.58 ± 17.95* | 8.58 ± 2.46* |

### Example 9: Therapeutic Effect of NGF-Fc Fusion Protein on Premature Ovarian Failure

Premature ovarian failure (POF) refers to natural amenorrhea before 40 years old caused by ovarian function failure, which is often accompanied by decrease of estrogen level, increase of follicle forming hormone level, and increase of gonadotropin level, with complex etiology and mechanism. This Example demonstrates the therapeutic efficacy of NGF-Fc fusion protein in treating POF via in vitro human ovarian granulosa-like tumor cell line (KGN) proliferation assay and KGN estrogen secretion assay, as well as in POF model rats.

In KGN proliferation assay, 100 μl KGN suspension (1×10⁴ cells/mL) was added to each well of 96-well plate one day before assay. Serum-free DMEM medium was replaced before experiment. After medium change, SuTaiSheng® mouse NGF, mNGF118, or NGF-Fc fusion protein (2-118-L3Fc10-M3-5 or 2-118-L3G4-BM) was added to experimental group wells, with the final concentration of 10 μg/mL. Negative control group was without any treatment after medium change. Each group has 4 replicates. After 48 hrs, 10 μL Cell Counting Kit-8 (DOJINDO, #CK04) was added to each well to measure viable cells. After one hour incubation, absorbance at 450 nm was measured. Recorded data were analyzed by Student t test. Histogram was drawn by GraphPad Prism 8.0.1.

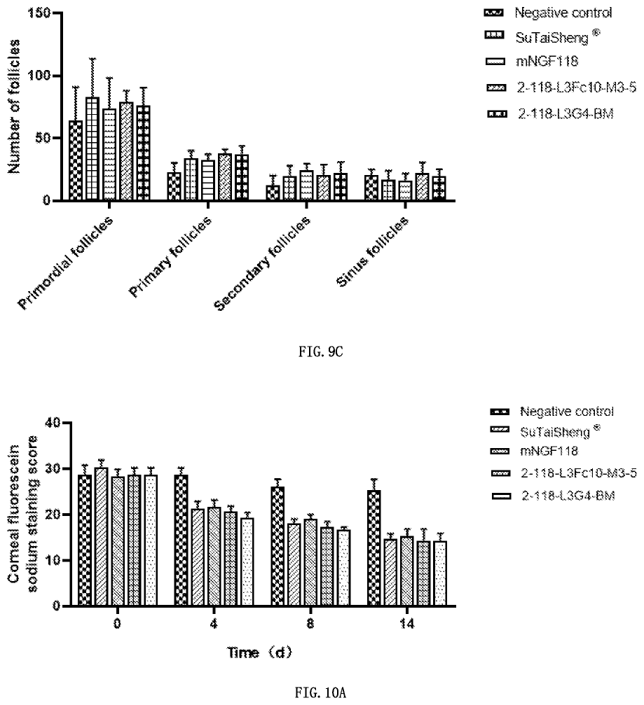

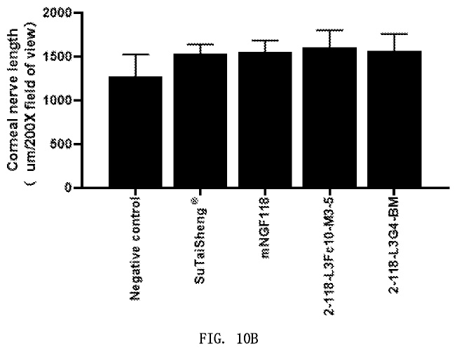

As shown in FIG. 9A, both NGF (SuTaiSheng® mouse NGF or mNGF118) and NGF-Fc fusion protein (2-118-L3Fc10-M3-5 or 2-118-L3G4-BM) significantly promoted KGN proliferation compared to negative control group (p<0.05). Relatively speaking, KGN treated with NGF-Fc fusion protein (2-118-L3Fc10-M3-5 or 2-118-L3G4-BM) showed slightly higher proliferation rate compared to KGN treated with NGF.

In KGN estrogen secretion assay, cells (the confluence was about 80%) were inoculated in 24-well plates with 1×10⁵ cells/well density, then serum-free medium was replaced. After medium change, SuTaiSheng® mouse NGF, mNGF118, or NGF-Fc fusion protein (2-118-L3Fc10-M3-5 or 2-118-L3G4-BM) was added to experimental group wells, with a final concentration of 10 μg/mL. Negative control group was without any treatment after medium change. Each group has 4 replicates. After 18 hours of incubation, cells were washed twice, then treated with 2.2×10⁻⁸ M testosterone (Beijing Solarbio Life Science & Technology Co., Ltd, #IT0110) and 0.01 IU/ml ovine follicle stimulating hormone (National Health Physics Program, Ovine FSH) for 24 hrs. The supernatant was obtained and diluted 1.6 times. By using the estrogen determination kit (KGE014) produced by R&D systems, the absorbance value at 450 nm was measured, and secreted estrogen concentration was calculated. Recorded data were analyzed by Student t test. Histogram was drawn using GraphPad Prism 8.0.1.

As shown in FIG. 9B, both NGF (SuTaiSheng® mouse NGF or mNGF118) and NGF-Fc fusion protein (2-118-L3Fc10-M3-5 or 2-118-L3G4-BM) significantly promoted estrogen secretion of KGN compared to negative control group (p<0.05). Relatively speaking, KGN treated with NGF-Fc fusion protein (2-118-L3Fc10-M3-5 or 2-118-L3G4-BM) showed slightly higher estrogen secretion compared to KGN treated with NGF.

4-vinylcyclohexene diepoxide (VCD) can selectively destroy primordial follicles and primary follicles in ovary of female rats, but has no effect on secondary follicles and sinus follicles, resulting in POF of female rats. To further study the in vivo efficacy of NGF-Fc fusion protein in treating POF, POF rat model was established by intraperitoneally injecting VCD to SD rats for two weeks consecutively (for example, see F. S. Muhammad et al., “Effects of 4-vinylcyclohexene diepoxide on peripubertal and adult Sprague-Dawley rats: ovarian, clinical, and pathologic outcomes.” Comp Med, 2009, 59 (1): 46-59). NGF treatment began when establishing animal model was started, denoted as Day 1. NGF (SuTaiSheng® mouse NGF or mNGF118) or NGF-Fc fusion protein (2-118-L3Fc10-M3-5 or 2-118-L3G4-BM) was subcutaneously administered at a dose of 10 μg/kg bw/injection for the experimental groups; equal volume of sterile saline solution was administrated to the negative control group. SuTaiSheng® mouse NGF, mNGF118 or sterile saline solution was administrated quaque die alterna (every other day). NGF-Fc fusion protein (2-118-L3Fc10-M3-5 or 2-118-L3G4-BM) was administrated once per week. 42 days later, all rats were sacrificed, ovarian tissues were obtained and fixed, embedded in paraffin, sectioned, and stained with H&E (hematoxylin and eosin). The numbers of follicles were then counted. Recorded data were analyzed by Student t test. Histogram was drawn using GraphPad Prism 8.0.1.

As shown in FIG. 9C, SuTaiSheng® mouse NGF, mNGF118, and NGF-Fc fusion protein (2-118-L3Fc10-M3-5 or 2-118-L3G4-BM) all significantly increased the number of primary follicles (p<0.05) compared to negative control group, demonstrating their superior effect in reversing the reduction of the number of primary follicles caused by POF. POF rat model treated with SuTaiSheng® mouse NGF, mNGF118, or NGF-Fc fusion protein (2-118-L3Fc10-M3-5 or 2-118-L3G4-BM) showed higher number of primordial follicles and secondary follicles as well, compared to negative control.

### Example 10: Therapeutic Effect of NGF-Fc Fusion Protein on Oligoasthenospermia

Oligoasthenospermia is mainly characterized by the reduction of sperm quantity and/or sperm motility. Sperm is generated through a series of division and differentiation events from germ cells with proliferative ability in testicular seminiferous tubules. Heat stress can affect division, differentiation, and sperm formation of germ cells. This Example illustrates the study of the therapeutic effect of NGF-Fc fusion protein on oligoasthenospermia (oligozoospermia and asthenospermia) in a mouse model of spermatogenesis disorder.

C57BL/6JSHjh mice (Shanghai Jihui Experimental Animal Breeding Co., Ltd) were used in the experiment. For experimental groups, 20 μg/kg bw/injection NGF (SuTaiSheng® mouse NGF or mNGF118) or 60 μg/kg bw/injection NGF-Fc fusion protein (2-118-L3Fc10-M3-5 or 2-118-L3G4-BM) was subcutaneously injected to the groin. Normal control group or spermatogenesis disorder model control group was treated by equal volume of 0.9% sodium chloride injection. The first day of treatment (NGF, NGF-Fc fusion protein, or sodium chloride) was denoted as Day 1. To establish mouse model of spermatogenesis disorder induced by heat stress, treated mice were anesthetized 4 hrs after the first injection. After mouse testis descended to the scrotum, the lower abdomen (hind limb, tail, and scrotum) of mice in the spermatogenesis disorder model control group, NGF experimental group, and NGF-Fc fusion protein experimental group was immersed in 42° C. constant temperature water bath for 30 minutes, while the lower abdomen of mice in normal control group was immersed in 25° C. constant temperature water bath for 30 minutes. SuTaiSheng® mouse NGF or mNGF118 was administered quaque die alterna. 2-118-L3G4-BM or 2-118-L3Fc10-M3-5 was administered twice per week. Normal control group or spermatogenesis disorder model control group was administered with 0.9% sodium chloride injection quaque die alterna. The total administration period was five weeks. All mice were sacrificed at Day 37. The left epididymal tail was harvested, weighed, then put into M199 culture medium preheated at 37° C., cut into pieces, and put into an incubator at 37° C. for 5 minutes. The sperm suspension was obtained and diluted with M199 culture medium at a ratio of 1:6. After mixing, the diluent was obtained, and the number and motility of sperms were recorded with TOXIVOS Sperm Analyzer. Recorded data were analyzed by Student t test.

As shown in Table 7, the number and motility of sperms of spermatogenesis disorder model control group were significantly lower than that of normal control group, demonstrating that the animal models were established successfully. The number and motility of sperms in NGF (SuTaiSheng® mouse NGF or mNGF118) and NGF-Fc fusion protein (2-118-L3G4-BM or 2-118-L3Fc10-M3-5) experimental groups significantly increased compared to spermatogenesis disorder model control group. These results demostrated that subcutaneous injection of NGF (SuTaiSheng® mouse NGF or mNGF118) or NGF-Fc fusion protein (2-118-L3G4-BM or 2-118-L3Fc10-M3-5) can effectively rescue the reduction of sperm number and poor sperm motility in spermatogenic disorder such as oligozoospermia, asthenospermia, and oligoasthenospermia.

TABLE 7
The number of sperms and sperm motility in
mouse model with spermatogenic disorder
treated with NGF or NGF-Fc fusion protein

| | Number of sperms | Sperm motility |
|---|---|---|
| Group | (×10⁶/g) | (%) |
| Normal control | 4222.5 ± 592.1 | 79.7 ± 5.3 |
| Spermatogenesis disorder | 1905.3 ± 747.2 | 59.5 ± 11.3 |
| model control | | |
| SuTaiSheng ® mouse NGF | 3201.6 ± 1246.0 | 71.5 ± 20.4 |
| mNGF118 | 2882.5 ± 1093.6 | 69.6 ± 10.9 |
| 2-118-L3Fc10-M3-5 | 3734.1 ± 1290.2 | 77.0 ± 11.3 |
| 2-118-L3G4-BM | 3544.9 ± 1442.9 | 73.8 ± 9.1 |

To further study the therapeutic effects of NGF and NGF-Fc fusion protein, the right testis and epididymis of the sacrificed mice were weighed, fixed with 10% neutral formalin, embedded, sectioned, and stained with H&E to evaluate their histopathology. As shown in Table 8, NGF (SuTaiSheng® mouse NGF or mNGF118) and NGF-Fc fusion protein (2-118-L3G4-BM or 2-118-L3Fc10-M3-5) demonstrate significant therapeutic effects on testicular seminiferous tubule atrophy, seminiferous tubule spermatogenesis disorder, and epididymal duct cell fragments caused by heat stress.

Further, NGF-Fc fusion protein demonstrated comparable or even better therapeutic effects (sperm number, sperm mobility, histopathology) than NGF.

TABLE 8
Histopathological statistics of subcutaneous injection of
NGF or NGF-Fc fusion protein in spermatogenic disorder mice

| | | | Normal | Model | SuTaiSheng ® | | 2-118-L3Fc10- | 2-118- |
|---|---|---|---|---|---|---|---|---|
| Group | | | control | control | mouse NGF | mNGF118 | M3-5 | L3G4-BM |
| Testis | Seminiferous | Slight | 1 | 4 | 4 | 3 | 2 | 3 |
| | tubule | Mild | 0 | 4 | 3 | 3 | 1 | 1 |
| | atrophy | Moderate | 0 | 2 | 1 | 2 | 0 | 1 |
| | | Severe | 0 | 1 | 0 | 0 | 0 | 0 |
| | | Total | 1 | 11 | 8 | 8 | 3 | 5 |
| | Seminiferous | Slight | 0 | 10 | 8 | 10 | 6 | 4 |
| | tubule | Mild | 0 | 2 | 2 | 1 | 1 | 1 |
| | spermatogenesis | Moderate | 0 | 1 | 0 | 0 | 0 | 0 |
| | disorder | Total | 0 | 13 | 10 | 11 | 7 | 5 |
| Epididymis | Epididymal | Slight | 0 | 7 | 8 | 9 | 10 | 9 |
| | spermatozoa | Mild | 0 | 3 | 2 | 1 | 0 | 0 |
| | | Total | 0 | 10 | 10 | 10 | 10 | 9 |
| | Epididymal | Slight | 1 | 5 | 3 | 2 | 1 | 1 |
| | duct cell | Total | 1 | 5 | 3 | 2 | 1 | 1 |
| | fragments | | | | | | | |
| | Sperm | Mild | 1 | 0 | 1 | 0 | 1 | 1 |
| | granuloma/ | Total | 1 | 0 | 1 | 0 | 1 | 1 |
| | seminoma | | | | | | | |

### Example 11: Therapeutic Effect of NGF-Fc Fusion Protein on Neurotrophic Keratitis

Neurotrophic keratitis is a degenerative disease caused by corneal epithelial healing disorder, which is mainly characterized by decreased corneal sensitivity. This Example illustrates the study of the therapeutic effects of NGF-Fc fusion protein on neurotrophic keratitis in a neurotrophic keratitis rat model (e.g., by corneal fluorescein sodium staining assay or corneal nerve length measurement).

To establish the neurotrophic keratitis animal model, 3-day-old SD rats were subcutaneously injected with 8 mg/ml capsaicin solution (Shanghai McLean Biochemical Technology Co., Ltd., #C10831884) at a dose of 50 μl per rat. Two weeks after capsaicin injection, 60 μg/ml NGF (SuTaiSheng® mouse NGF or mNGF118) or NGF-Fc fusion protein (2-118-L3G4-BM or 2-118-L3Fc10-M3-5) was administered 6 times a day with an interval of about 2 hours in the form of eye drops, 20 μl/eye/administration. Equal volume of 0.9% sodium chloride solution was administered at the same frequency as negative control. The first day of administration was denoted as D1. The treatment lasted for 2 weeks, and was performed only once in each group on D15.

Corneal fluorescein sodium staining was then carried out to evaluate the therapeutic effects of NGF-Fc fusion protein. Corneal fluorescein sodium staining score can directly indicate the integrity and damage degree of cornea. Intact cornea cannot be stained. Only damaged cornea can be stained, and the higher the staining score, the higher the degree of corneal damage. Briefly, the fluorescein sodium solution (3 μL, 0.5%) was dropped into rats' eyes, and the eyes were stained for 1.5 min. Then the conjunctival sac was rinsed with 1.25 mL sterile normal saline every 10 seconds for 3 consecutive times. After each wash, the normal saline around eyes was sopped up with paper tissue. 5 minutes after staining, the ocular surface was observed in slit lamp (with cobalt blue filter), and photos were taken and scored. An improved NEI scale for grading fluorescent staining was used as the scoring standard. Specifically, each cornea was divided into 5 areas (1-central area, 2-upper, 3-temporal, 4-nasal, and 5-lower), the top score for each area was 8 points, wherein point 0 indicated no colored area, 1 indicated the spot colored area was 1%˜ 25% of the corresponding area, 2 indicated the spot colored area was 26%˜ 50% of the corresponding area, 3 indicated the spot colored area was 51%˜ 75% of the corresponding area, and 4 indicated the spot colored area was 76%˜ 100% of the corresponding area. If the colored area was dense and/or obvious fusion in the area can be seen, 1, 2, 3, or 4 points would be further given according to the percentage of the colored area in the corresponding area, respectively, i.e. 1 extra point for colored area of 1%˜ 25%, 2 extra points for colored area of 26%˜ 50%, 3 extra points for colored area of 51%˜ 75%, and 4 extra points for colored area of 76%˜ 100%. The maximum total score of each eye was 40 points. The measurements were taken 4 times in total on Days 0 (before the first treatment), 4, 8, and 14. Total score of corneal fluorescein sodium staining was calculated. Recorded data were analyzed by SPSS13.0, and histogram was drawn using GraphPad Prism 8.0.1.

As shown in FIG. 10A, the corneal fluorescein sodium staining scores of neurotrophic keratitis model rats in NGF (SuTaiSheng® mouse NGF or mNGF118) or NGF-Fc fusion protein (2-118-L3G4-BM or 2-118-L3Fc10-M3-5) treated experimental group were significantly lower (p<0.01 on Day 4 and 8, p<0.001 on Day 14) than that in the negative control group, indicating that NGF (SuTaiSheng® mouse NGF or mNGF118) or NGF-Fc fusion protein (2-118-L3G4-BM or 2-118-L3Fc10-M3-5) can significantly restore the integrity of damaged cornea. Relatively speaking, NGF-Fc fusion protein (2-118-L3Fc10-M3-5 or 2-118-L3G4-BM) treated cornea showed slightly lower fluorescein sodium staining scores compared to NGF treated cornea.

A corneal nerve counting assay was further carried out to study the therapeutic efficacy. On D15, rats were sacrificed 1 hour after the last treatment. The right eyeball was harvested. Cornea was separated along the corneal limbus, washed, paved, stained, and fixed on glass slides. The morphology of corneal nerve fibers was observed under optical microscope (200×). The area of cornea was divided radially from the center into four flaps, followed by taking photos of a vision field with the most corneal nerves in each of the four flaps. Then, the length of corneal nerves in each vision field was measured, and the average length of corneal nerves in all four fields was calculated as the final result. SPSS13.0 was used to process the data, and graphpad prism 8.0.1 was used to draw the histogram.

As shown in FIG. 10B, the average corneal nerve length of rat models of neurotrophic keratitis treated with NGF (SuTaiSheng® mouse NGF or mNGF118) or NGF-Fc fusion protein (2-118-L3G4-BM or 2-118-L3Fc10-M3-5) was significantly longer (p<0.05) than that in the negative control group. Specifically, the average corneal nerve length of rats treated by SuTaiSheng® mouse NGF, mNGF118, 2-118-L3G4-BM, and 2-118-L3Fc10-M3-5 was about 1.14 times, 1.14 times, 1.21 times, and 1.18 times of that in negative control group, respectively. These results demonstrate that NGF (SuTaiSheng® mouse NGF or mNGF118) or NGF-Fc fusion protein (2-118-L3G4-BM or 2-118-L3Fc10-M3-5) can effectively remedy the damage of corneal nerve caused by neurotrophic keratitis.

SEQUENCE LISTING

SEQ ID NO: 1 (mutant human β-NGF, 118aa)
SSSHPIFHRGEESVCDSVSVWVGDKTTATDIKGKEVMVLGEVNINNSVFKQYFFETKCRDPNP
VDSGCRGIDSKHWNSYCTTTHTFVKALTMDGKQAAWRFIRIDTACVCVLSRKAVR
SEQ ID NO: 2 (mutant human β-NGF, 120aa)
SSSHPIFHRGEESVCDSVSVWVGDKTTATDIKGKEVMVLGEVNINNSVFKQYFFETKCRDPNP
VDSGCRGIDSKHWNSYCTTTHTFVKALTMDGKQAAWRFIRIDTACVCVLSRKAVRRA
SEQ ID NO: 3 (wildtype human β-NGF, 118aa)
SSSHPIFHRGEFSVCDSVSVWVGDKTTATDIKGKEVMVLGEVNINNSVFKQYFFETKCRDPNP
VDSGCRGIDSKHWNSYCTTTHTFVKALTMDGKQAAWRFIRIDTACVCVLSRKAVR
SEQ ID NO: 4 (wildtype human β-NGF, 120aa)
SSSHPIFHRGEFSVCDSVSVWVGDKTTATDIKGKEVMVLGEVNINNSVFKQYFFETKCRDPNP
VDSGCRGIDSKHWNSYCTTTHTFVKALTMDGKQAAWRFIRIDTACVCVLSRKAVRRA
SEQ ID NO: 5 (NGF propeptide, 103aa)
EPHSESNVPAGHTIPQAHWTKLQHSLDTALRRARSAPAAAIAARVAGQTRNITVDPRLFKKRR
LRSPRVLFSTQPPREAADTQDLDFEVGGAAPFNRTHRSKR
SEQ ID NO: 6 (NGF signal peptide, 18aa)
MSMLFYTLITAFLIGIQA
SEQ ID NO: 7 (human wildtype IgG1 Fc IGHG1*05)
EPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK
SEQ ID NO: 8 (human wildtype IgG1 Fc IGHG1*03, natural variant)
EPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 9 (modified IgG1 Fc M1 [N297A relative to IGHG1*03])
EPKSCDKTHTCPPCPAPELLGGPSVFLFPPKPKDTLMISRTPEVTCVVVDVSHEDPEVKFNWY
VDGVEVHNAKTKPREEQYASTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 10 (modified IgG1 Fc M1-5 [N297A relative to
IGHG1*03, N′ 5aa truncation])
DKTHTCPPCPAPELLGGPSVFLFPPKPKDTLMISRTPEVTCVVVDVSHEDPEVKFNWYVDGVE
VHNAKTKPREEQYASTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 11 (modified IgG1 Fc M3 [L234A+L235A+P331S
relative to IGHG1*03])
EPKSCDKTHTCPPCPAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPASIEKTISKA
KGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 12 (modified IgG1 Fc M3-5 [L234A+L235A+P331S
relative to IGHG1*03, N′ 5aa truncation])
DKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPASIEKTISKAKGQPR
EPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 13 (modified IgG1 Fc M5 [L234A+L235E+G237A+A330S+P331S
relative to IGHG1*03])
EPKSCDKTHTCPPCPAPEAEGAPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPSSIEKTISKA
KGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 14 (modified IgG1 Fc M5-5 [L234A+L235E+G237A+A330S+P331S
relative to IGHG1*03, N′ 5aa truncation])
DKTHTCPPCPAPEAEGAPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPSSIEKTISKAKGQPR
EPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 15 (modified IgG1 Fc M7 [E233P+L234V+L235A+G236del+
A327G+A330S+P331S relative to IGHG1*03])
EPKSCDKTHTCPPCPAPPVAGPSVFLFPPKPKDTLMISRTPEVTCVVVDVSHEDPEVKFNWYV
DGVEVHNAKTKPREEQYNSTYRVVSVLTVLHQDWLNGKEYKCKVSNKGLPSSIEKTISKAK
GQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 16 (modified IgG1 Fc M7-5[E233P+L234V+L235A+G236del+A327G+A330S+P331S
relative to IGHG1*03, N′ 5aa truncation])
DKTHTCPPCPAPPVAGPSVFLFPPKPKDTLMISRTPEVTCVVVDVSHEDPEVKFNWYVDGVEV
HNAKTKPREEQYNSTYRVVSVLTVLHQDWLNGKEYKCKVSNKGLPSSIEKTISKAKGQPREP
QVYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGK
SEQ ID NO: 17 (human wildtype IgG4 Fc)
ESKYGPPCPSCPAPEFLGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK
SEQ ID NO: 18 (modified IgG4 Fc [S228P+F234A+L235A])
ESKYGPPCPPCPAPEAAGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK
SEQ ID NO: 19 (modified IgG4 Fc-FD)
SKYGPPCPPCPAPEFEGGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLY
SRLTVDKSRWQEGNVFSCSVMHEALHNHYTQKSLSLSPGK
SEQ ID NO: 20 (modified IgG2/4 Fc)
VERKCCVECPPCPAPPVAGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNYKTTPPVLDSDGSFF
LYSRLTVDKSRWQEGNVFSCSVMHEALHNHYTQKSLSLSLGK
SEQ ID NO: 21 (nucleic acid sequence of FD-G4Fc; signal peptide
is italicized, propeptide is squared, β-NGF (wt 118aa)
is bolded, linker is underlined, modified IgG4 Fc is italicized
and bolded)

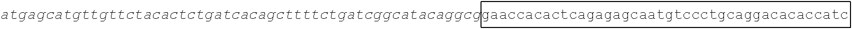

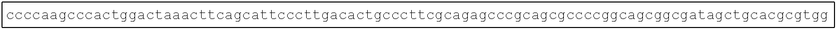

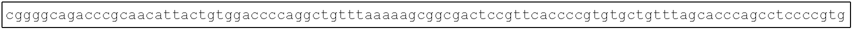

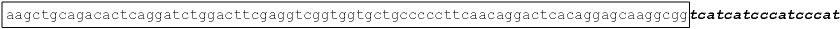

cttccacaggggcgaattctcggtgtgtgacagtgtcagcgtgtgggttggggataagaccaccgccacagacatcaagggcaaggagg
tgatggtgttgggagaggtgaacattaacaacagtgtattcaaacagtacttttttgagaccaagtgccgggacccaaatcccgttgacag
cgggtgccggggcattgactcaaagcactggaactcatattgtaccacgactcacacctttgtcaaggcgctgaccatggatggcaagca
ggctgcctggcggtttatccggatagatacggcctgtgtgtgtgtgctcagcaggaaggctgtgagaGGCTCCGGCGGCGGCT
CCGGTGGCGGCGGCTCAGGAGGAGGAGGCTCCGGTGGTGGTGGTTCCTCCAAGTATGGCC
CCCCCTGCCCCCCCTGCCCAGCACCTGAGTTCGAGGGGggaccatcagtcttcctgttccccccaaaacccaa
ggacactctcatgatctcccggacccctgaggtcacgtgcgtggtggtggacgtgagccaggaagaccccgaggtccagttcaactggtacgt
ggatggcgtggaggtgcataatgccaagacaaagccgcgggaggagcagttcaacagcacgtaccgtgtggtcagegtcctcaccgtcctg
caccaggactggctgaacggcaaggagtacaagtgcaaggtctccaacaaaggcctcccgtcctccategagaaaaccatctccaaagcc
aaagggcagccccgagagccacaggtgtacaccctgcccccatcccaggaggagatgaccaagaaccaggtcagcctgacctgcctggtc
aaaggcttctaccccagcgacatcgccgtggagtgggaaagcaatgggcagccggagaacaactacaagaccacgcctcccgtgctggac
tccgacggctccttcttcctctacagcaggctaaccgtggacaagagcaggtggcaggaggggaatgtcttctcatgctccgtgatgcatgagg
ctctgcacaaccactacacacagaagagcctctccctgtctCCGggtaaa
SEQ ID NO: 22 (amino acid sequence of FD-G4Fc; signal peptide is italicized, propeptide is
squared, β-NGF (wt 118aa) is bolded, linker is underlined, modified IgG4 Fc is italicized and
bolded)

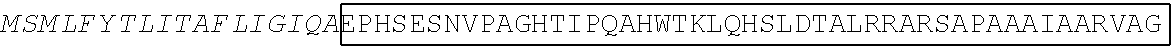

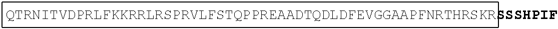

HRGEFSVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVRGSGGGS
GGGGSGGGGSGGGGSSKYGPPCPPCPAPEFEGGPSVFLFPPKPKDTLMISRTPEVTCVVVDV
SQEDPEVQFNWYVDGVEVHNAKTKPREEQFNSTYRVVSVLTVLHQDWLNGKEYKCKVSNK
GLPSSIEKTISKAKGQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQKSLSLSPGK
SEQ ID NO: 23 (nucleic acid sequence of WM-G24Fc; signal peptide is italicized, propeptide is
squared, β-NGF (wt 118aa) is bolded, linker is underlined, modified IgG2/4 Fc is italicized and
bolded)

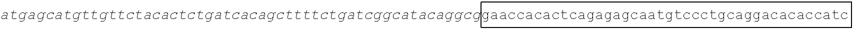

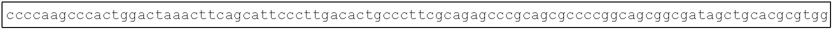

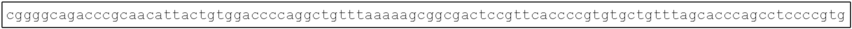

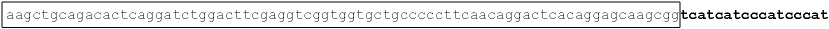

cttccacaggggcgaattctcggtgtgtgacagtgtcagcgtgtgggttggggataagaccaccgccacagacatcaagggcaaggagg
tgatggtgttgggagaggtgaacattaacaacagtgtattcaaacagtacttttttgagaccaagtgccgggacccaaatcccgttgacag
cgggtgccggggcattgactcaaagcactggaactcatattgtaccacgactcacacctttgtcaaggcgctgaccatggatggcaagca
ggctgcctggcggtttatccggatagatacggcctgtgtgtgtgtgctcagcaggaaggctgtgagaAAGACCGGCGGTGGC
TCCGGCGGCGGCTCCGTGGAGCGGAAGTGCTGCGTGGAGTGCCCCCCCTGCCCCGCTCCC
CCCGTGGCTGGACCATCAGTCTTCCTGTTCCCCCCAAAACCCAAGGACACTCTCATGATCTC
CCGGACCCCTGAGGTCACGTGCGTGGTGGTGGACGTGAGCCAGGAAGACCCCGAGGTCCA
GTTCAACTGGTACGTGGATGGCGTGGAGGTGCATAATGCCAAGACAAAGCCGCGGGAGGA
GCAGTTCAACAGCACGTACCGTGTGGTCAGCGTCCTCACCGTCCTGCACCAGGACTGGCTG
AACGGCAAGGAGTACAAGTGCAAGGTCTCCAACAAAGGCCTCCCGTCCTCCATCGAGAAAA
CCATCTCCAAAGCCAAAGGGCAGCCCCGAGAGCCACAGGTGTACACCCTGCCCCCATCCCA
GGAGGAGATGACCAAGAACCAGGTCAGCCTGACCTGCCTGGTCAAAGGCTTCTACCCCAGC
GACATCGCCGTGGAGTGGGAAAGCAATGGGCAGCCGGAGAACAACTACAAGACCACGCCT
CCCGTGCTGGACTCCGACGGCTCCTTCTTCCTCTACAGCAGGCTAACCGTGGACAAGAGCA
GGTGGCAGGAGGGGAATGTCTTCTCATGCTCCGTGATGCATGAGGCTCTGCACAACCACTA
CACACAGAAGAGCCTCTCCCTGTCTCTGGGTAAA
SEQ ID NO: 24 (amino acid sequence of WM-G24Fc; signal peptide is italicized, propeptide is
squared, β-NGF (wt 118aa) is bolded, linker is underlined, modified IgG2/4 Fc is italicized and
bolded)

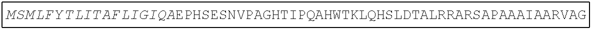

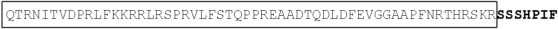

HRGEFSVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVRKTGGGS
GGGSVERKCCVECPPCPAPPVAGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
SEQ ID NO: 25 (nucleic acid sequence of NGF-1-15M7; signal peptide is italicized, propeptide is
squared, β-NGF (wt 120aa) is bolded, modified IgG1 Fc is italicized and bolded)

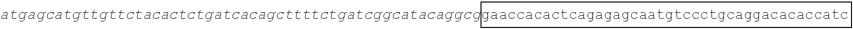

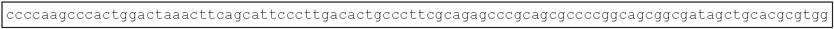

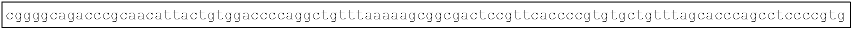

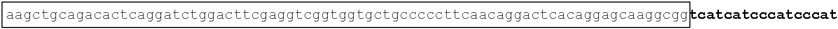

cttccacaggggcgaattctcggtgtgtgacagtgtcagcgtgtgggttggggataagaccaccgccacagacatcaagggcaaggagg
tgatggtgttgggagaggtgaacattaacaacagtgtattcaaacagtacttttttgagaccaagtgccgggacccaaatcccgttgacag
cgggtgccggggcattgactcaaagcactggaactcatattgtaccacgactcacacctttgtcaaggcgctgaccatggatggcaagca
ggctgcctggcggtttatccggatagatacggcctgtgtgtgtgtgctcagcaggaaggctgtgagaagagccGAGCCCAAATCT
TGTGACAAAACTCACACATGCCCACCGTGCCCAGCACCTCCAGTCGCAGGACCGTCAGTCT
TCCTCTTCCCCCCAAAACCCAAGGACACCCTCATGATCTCCCGGACCCCTGAGGTCACATG
CGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCG
TGTGGTCAGCGTCCTCACCGTCCTGCACCAGGACTGGCTGAATGGCAAGGAGTACAAGTGC
AAGGTCTCCAACAAAGGCCTCCCATCCTCCATCGAGAAAACCATCTCCAAAGCCAAAGGGC
AGCCCCGAGAACCACAGGTGTACACCCTGCCCCCATCCCGGGAGGAGATGACCAAGAACC
AGGTCAGCCTGACCTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGG
CTCCTTCTTCCTCTATAGCAAGCTCACCGTGGACAAGAGCAGGTGGCAGCAGGGGAACGTC
TTCTCATGCTCCGTGATGCATGAGGCTCTGCACAACCACTACACGCAGAAGAGCCTCTCCCT
GTCCCCGGGTAAA
SEQ ID NO: 26 (amino acid sequence of NGF-1-15M7; signal peptide is italicized, propeptide is
squared, β-NGF (wt 120aa) is bolded, modified IgG1 Fc is italicized and bolded)

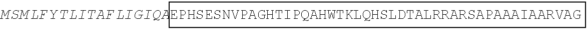

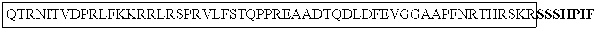

HRGEFSVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVRRAEPKSC
DKTHTCPPCPAPPVAGPSVFLFPPKPKDTLMISRTPEVTCVVVDVSHEDPEVKFNWYVDGVE
VHNAKTKPREEQYNSTYRVVSVLTVLHQDWLNGKEYKCKVSNKGLPSSIEKTISKAKGQPREP
QVYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGK
SEQ ID NO: 27 (nucleic acid sequence of NGF-L3Fc10M7-5; signal peptide is italicized,
propeptide is squared, β-NGF (wt 120aa) is bolded, linker is underlined, modified IgG1 Fc is
italicized and bolded)

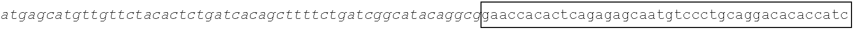

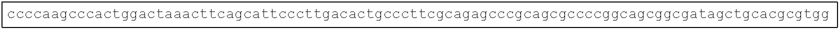

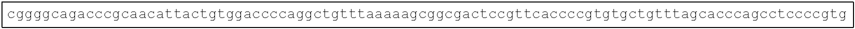

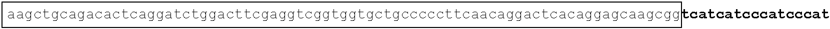

cttccacaggggcgaattctcggtgtgtgacagtgtcagcgtgtgggttggggataagaccaccgccacagacatcaagggcaaggagg
tgatggtgttgggagaggtgaacattaacaacagtgtattcaaacagtacttttttgagaccaagtgccgggacccaaatcccgttgacag
cgggtgccggggcattgactcaaagcactggaactcatattgtaccacgactcacacctttgtcaaggcgctgaccatggatggcaagca
ggctgcctggcggtttatccggatagatacggcctgtgtgtgtgtgctcagcaggaaggctgtgagaagagccggcggtggcggctccgg
cggtggcggctccggcggtggcggctccGACAAAACTCACACATGCCCACCGTGCCCAGCACCTCCTGTC
GCCGGACCGTCAGTCTTCCTCTTCCCCCCAAAACCCAAGGACACCCTCATGATCTCCCGGA
CCCCTGAGGTCACATGCGTGGTGGTGGACGTGAGCCACGAAGACCCTGAGGTCAAGTTCA
ACTGGTACGTGGACGGCGTGGAGGTGCATAATGCCAAGACAAAGCCGCGGGAGGAGCAGT
ACAACAGCACGTACCGTGTGGTCAGCGTCCTCACCGTCCTGCACCAGGACTGGCTGAATGG
CAAGGAGTACAAGTGCAAGGTCTCCAACAAAGGCCTCCCATCCTCCATCGAGAAAACCATC
TCCAAAGCCAAAGGGCAGCCCCGAGAACCACAGGTGTACACCCTGCCCCCATCCCGGGAG
GAGATGACCAAGAACCAGGTCAGCCTGACCTGCCTGGTCAAAGGCTTCTATCCCAGCGACA
TCGCCGTGGAGTGGGAGAGCAATGGGCAGCCGGAGAACAACTACAAGACCACGCCTCCCG
TGCTGGACTCCGACGGCTCCTTCTTCCTCTATAGCAAGCTCACCGTGGACAAGAGCAGGTG
GCAGCAGGGGAACGTCTTCTCATGCTCCGTGATGCATGAGGCTCTGCACAACCACTACACG
CAGAAGAGCCTCTCCCTGTCCCCGGGTAAA
SEQ ID NO: 28 (amino acid sequence of NGF-L3Fc10M7-5; signal peptide is italicized,
propeptide is squared, β-NGF (wt 120aa) is bolded, linker is underlined, modified IgG1 Fc is
italicized and bolded)

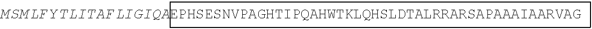

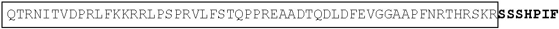

HRGEFSVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVRRAGGGG
SGGGGSGGGGSDKTHTCPPCPAPPVAGPSVFLFPPKPKDTLMISRTPEVTCVVVDVSHEDPE
VKFNWYVDGVEVHNAKTKPREEQYNSTYRVVSVLTVLHQDWLNGKEYKCKVSNKGLPSSIE
KTISKAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
SEQ ID NO: 29 (nucleic acid sequence of 2-1-15M7; signal peptide is italicized, propeptide is
squared, β-NGF (mutant 120aa) is bolded, modified IgG1 Fc is italicized and bolded)

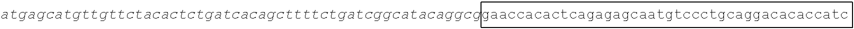

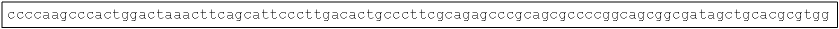

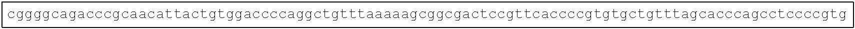

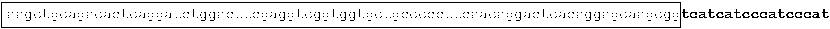

cttccacaggggcgaagagtcggtgtgtgacagtgtcagcgtgtgggttggggataagaccaccgccacagacatcaagggcaaggag
gtgatggtgttgggagaggtgaacattaacaacagtgtattcaaacagtacttttttgagaccaagtgccgggacccaaatcccgttgaca
gcgggtgccggggcattgactcaaagcactggaactcatattgtaccacgactcacacctttgtcaaggcgctgaccatggatggcaagc
aggctgcctggcggtttatccggatagatacggcctgtgtgtgtgtgctcagcaggaaggctgtgagaagagccGAGCCCAAATC
TTGTGACAAAACTCACACATGCCCACCGTGCCCAGCACCTCCAGTCGCAGGACCGTCAGTC
TTCCTCTTCCCCCCAAAACCCAAGGACACCCTCATGATCTCCCGGACCCCTGAGGTCACATG
CGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCG
TGTGGTCAGCGTCCTCACCGTCCTGCACCAGGACTGGCTGAATGGCAAGGAGTACAAGTGC
AAGGTCTCCAACAAAGGCCTCCCATCCTCCATCGAGAAAACCATCTCCAAAGCCAAAGGGC
AGCCCCGAGAACCACAGGTGTACACCCTGCCCCCATCCCGGGAGGAGATGACCAAGAACC
AGGTCAGCCTGACCTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGG
CTCCTTCTTCCTCTATAGCAAGCTCACCGTGGACAAGAGCAGGTGGCAGCAGGGGAACGTC
TTCTCATGCTCCGTGATGCATGAGGCTCTGCACAACCACTACACGCAGAAGAGCCTCTCCCT
GTCCCCGGGTAAA
SEQ ID NO: 30 (amino acid sequence of 2-1-15M7; signal peptide is italicized, propeptide is
squared, β-NGF (mutant 120aa) is bolded, modified IgG1 Fc is italicized and bolded)

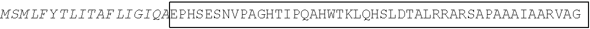

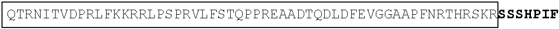

HRGEESVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVRRAEPKSC
DKTHTCPPCPAPPVAGPSVFLFPPKPKDTLMISRTPEVTCVVVDVSHEDPEVKFNWYVDGVE
VHNAKTKPREEQYNSTYRVVSVLTVLHQDWLNGKEYKCKVSNKGLPSSIEKTISKAKGQPREP
QVYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGK
SEQ ID NO: 31 (nucleic acid sequence of NGF-4-12PAA; signal peptide is italicized, propeptide
is squared, β-NGF (wt 120aa) is bolded, modified IgG4 Fc is italicized and bolded)

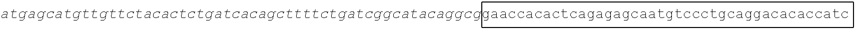

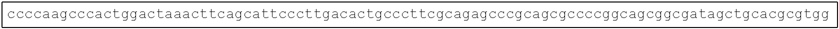

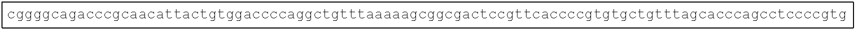

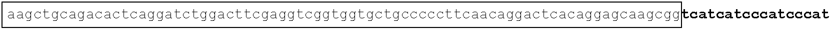

cttccacaggggcgaattctcggtgtgtgacagtgtcagcgtgtgggttggggataagaccaccgccacagacatcaagggcaaggagg
tgatggtgttgggagaggtgaacattaacaacagtgtattcaaacagtacttttttgagaccaagtgccgggacccaaatcccgttgacag
cgggtgccggggcattgactcaaagcactggaactcatattgtaccacgactcacacctttgtcaaggcgctgaccatggatggcaagca
ggctgcctggcggtttatccggatagatacggcctgtgtgtgtgtgctcagcaggaaggctgtgagaagagccgagtccaaatatggtccc
ccatgcccaccctgcccagcacctgaggctgcggggggaccatcagtcttcctgttccccccaaaacccaaggacactctcatgatctcccgg
acccctgaggtcacgtgcgtggtggtggacgtgagccaggaagaccccgaggtccagttcaactggtacgtggatggcgtggaggtgcata
atgccaagacaaagccgcgggaggagcagttcaacagcacgtaccgtgtggtcagcgtcctcaccgtcctgcaccaggactggctgaacg
gcaaggagtacaagtgcaaggtctccaacaaaggcctcccgtcctccategagaaaaccatctccaaagccaaagggcagccccgagag
ccacaggtgtacaccctgcccccatcccaggaggagatgaccaagaaccaggtcagcctgacctgcctggtcaaaggcttctaccccagcg
acatcgccgtggagtgggaaagcaatgggcagccggagaacaactacaagaccacgcctcccgtgctggactccgacggetccttettectc
tacagcaggctaaccgtggacaagagcaggtggcaggaggggaatgtcttctcatgctccgtgatgcatgaggctctgcacaaccactacac
acagaagagcctctccctgtctctgggtaaa
SEQ ID NO: 32 (amino acid sequence of NGF-4-12PAA; signal peptide is italicized, propeptide
is squared, β-NGF (wt 120aa) is bolded, modified IgG4 Fc is italicized and bolded)

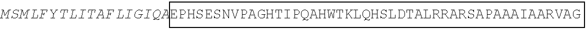

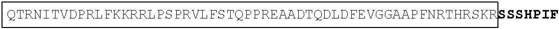

HRGEFSVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVRRAESKYG
PPCPPCPAPEAAGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK
SEQ ID NO: 33 (nucleic acid sequence of 2-118-L3Fc10-M1-5; signal peptide is italicized,
propeptide is squared, β-NGF (mutant 118aa) is bolded, linker is underlined, modified IgG1 Fc
is italicized and bolded)

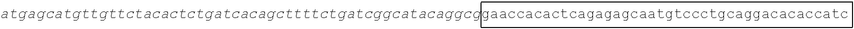

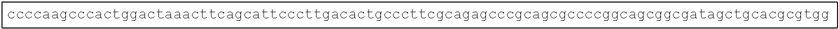

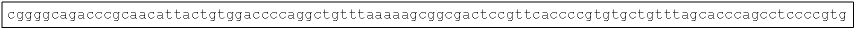

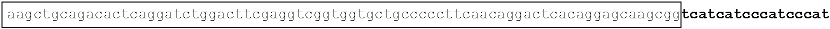

cttccacaggggcgaagagtcggtgtgtgacagtgtcagcgtgtgggttggggataagaccaccgccacagacatcaagggcaaggag
gtgatggtgttgggagaggtgaacattaacaacagtgtattcaaacagtacttttttgagaccaagtgccgggacccaaatcccgttgaca
gcgggtgccggggcattgactcaaagcactggaactcatattgtaccacgactcacacctttgtcaaggcgctgaccatggatggcaagc
aggctgcctggcggtttatccggatagatacggcctgtgtgtgtgtgctcagcaggaaggctgtgagaggcggtggcggctccggcggtgg
cggctccggcggtggcggctccGACAAAACTCACACATGCCCACCGTGCCCAGCACCTGAACTCCTGG
GGGGACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAA
CTGGTACGTGGACGGCGTGGAGGTGCATAATGCCAAGACAAAGCCGCGGGAGGAGCAGTA
CGCTAGCACGTACCGTGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCT
CCAAAGCCAAAGGGCAGCCCCGAGAACCACAGGTGTACACCCTGCCCCCATCCCGGGAGG
AGATGACCAAGAACCAGGTCAGCCTGACCTGCCTGGTCAAAGGCTTCTATCCCAGCGACAT
CGCCGTGGAGTGGGAGAGCAATGGGCAGCCGGAGAACAACTACAAGACCACGCCTCCCGT
GCTGGACTCCGACGGCTCCTTCTTCCTCTATAGCAAGCTCACCGTGGACAAGAGCAGGTGG
CAGCAGGGGAACGTCTTCTCATGCTCCGTGATGCATGAGGCTCTGCACAACCACTACACGC
AGAAGAGCCTCTCCCTGTCCCCGGGTAAA
SEQ ID NO: 34 (amino acid sequence of 2-118-L3Fc10-M1-5; signal peptide is italicized,
propeptide is squared, β-NGF (mutant 118aa) is bolded, linker is underlined, modified IgG1 Fc
is italicized and bolded)

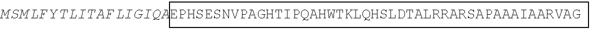

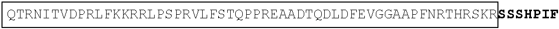

HRGEESVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVRGGGGSG
GGGSGGGGSDKTHTCPPCPAPELLGGPSVFLFPPKPKDTLMISRTPEVTCVVVDVSHEDPEV
KFNWYVDGVEVHNAKTKPREEQYASTYRVVSVLTVLHQDWLNGKEYKCKVSNKALPAPIEKT
ISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
SEQ ID NO: 35 (nucleic acid sequence of 2-118-L3Fc10-M3-5; signal peptide is italicized,
propeptide is squared, β-NGF (mutant 118aa) is bolded, linker is underlined, modified IgG1 Fc
is italicized and bolded)

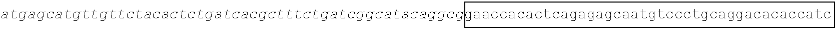

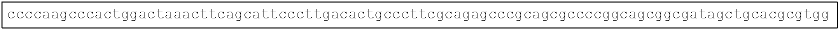

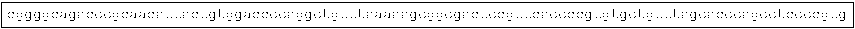

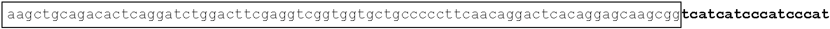

cttccacaggggcgaagagtcggtgtgtgacagtgtcagcgtgtgggttggggataagaccaccgccacagacatcaagggcaaggag
gtgatggtgttgggagaggtgaacattaacaacagtgtattcaaacagtacttttttgagaccaagtgccgggacccaaatcccgttgaca
gcgggtgccggggcattgactcaaagcactggaactcatattgtaccacgactcacacctttgtcaaggcgctgaccatggatggcaagc
aggctgcctggcggtttatccggatagatacggcctgtgtgtgtgtgctcagcaggaaggctgtgagaggcggtggcggctccggcggtgg
cggctccggcggtggcggctccgacaaaactcacacatgcccaccgtgcccagcacctgaagccgctgggggacegtcagtettcctettcc
ccccaaaacccaaggacaccctcatgatctcccggacccctgaggtcacatgcgtggtggtggacgtgagccacgaagaccctgaggtcaa
gttcaactggtacgtggacggcgtggaggtgcataatgccaagacaaagccgcgggaggagcagtacaacagcacgtaccgtgtggtcag
cgtcctcaccgtcctgcaccaggactggctgaatggcaaggagtacaagtgcaaggtctccaacaaagccctcccagcctccatcgagaaa
accatctccaaagccaaagggcagccccgagaaccacaggtgtacaccctgcccccatcccgggaggagatgaccaagaaccaggtcag
cctgacctgcctggtcaaaggcttctatcccagcgacatcgccgtggagtgggagagcaatgggcagccggagaacaactacaagaccac
gcctcccgtgctggactccgacggctccttcttcctctatagcaagctcaccgtggacaagagcaggtggcagcaggggaacgtcttctcatgc
tccgtgatgcatgaggctctgcacaaccactacacgcagaagagcctctccctgtccccgggtaaa
SEQ ID NO: 36 (amino acid sequence of 2-118-L3Fc10-M3-5; signal peptide is italicized,
propeptide is squared, β-NGF (mutant 118aa) is bolded, linker is underlined, modified IgG1 Fc
is italicized and bolded)

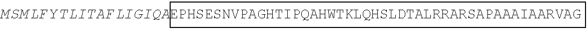

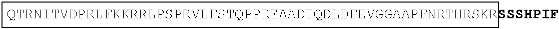

HRGEESVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVRGGGGSG
GGGSGGGGSDKTHTCPPCPAPEAAGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPASIEKT
ISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
SEQ ID NO: 37 (nucleic acid sequence of NGF-118-L3Fc10-M3-5; signal peptide is italicized,
propeptide is squared, β-NGF (wt 118aa) is bolded, linker is underlined, modified IgG1 Fc is
italicized and bolded)

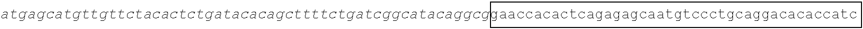

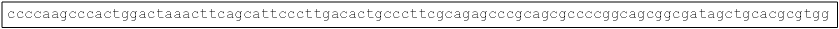

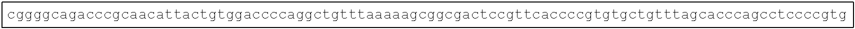

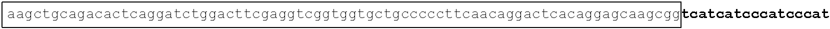

cttccacaggggcgaattctcggtgtgtgacagtgtcagcgtgtgggttggggataagaccaccgccacagacatcaagggcaaggagg
tgatggtgttgggagaggtgaacattaacaacagtgtattcaaacagtacttttttgagaccaagtgccgggacccaaatcccgttgacag
cgggtgccggggcattgactcaaagcactggaactcatattgtaccacgactcacacctttgtcaaggcgctgaccatggatggcaagca
ggctgcctggcggtttatccggatagatacggcctgtgtgtgtgtgctcagcaggaaggctgtgagaggcggtggcggctccggcggtggc
ggctccggcggtggcggctccgacaaaactcacacatgcccaccgtgcccagcacctgaagccgctgggggaccgtcagtettcctettccc
cccaaaacccaaggacaccctcatgatctcccggacccctgaggtcacatgcgtggtggtggacgtgagccacgaagaccctgaggtcaag
ttcaactggtacgtggacggcgtggaggtgcataatgccaagacaaagccgcgggaggagcagtacaacagcacgtaccgtgtggtcagc
gtcctcaccgtcctgcaccaggactggctgaatggcaaggagtacaagtgcaaggtctccaacaaagccctcccagcctccatcgagaaaa
ccatctccaaagccaaagggcagccccgagaaccacaggtgtacaccctgcccccatcccgggaggagatgaccaagaaccaggtcagc
ctgacctgcctggtcaaaggcttctatcccagcgacatcgccgtggagtgggagagcaatgggcagccggagaacaactacaagaccacg
cctcccgtgctggactccgacggctccttcttcctctatagcaagctcaccgtggacaagagcaggtggcagcaggggaacgtcttctcatgct
ccgtgatgcatgaggctctgcacaaccactacacgcagaagagcctctccctgtccccgggtaaa
SEQ ID NO: 38 (amino acid sequence of NGF-118-L3Fc10-M3-5; signal peptide is italicized,
propeptide is squared, β-NGF (wt 118aa) is bolded, linker is underlined, modified IgG1 Fc is
italicized and bolded)

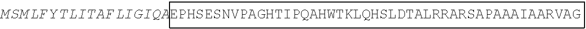

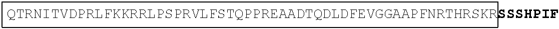

HRGEFSVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVRGGGGSG
GGGSGGGGSDKTHTCPPCPAPEAAGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPASIEKT
ISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
SEQ ID NO: 39 (nucleic acid sequence of 2-L3Fc10-M3-5; signal peptide is italicized,
propeptide is squared, β-NGF (mutant 120aa) is bolded, linker is underlined, modified IgG1 Fc
is italicized and bolded)

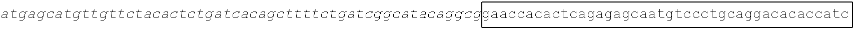

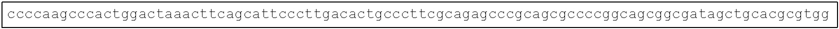

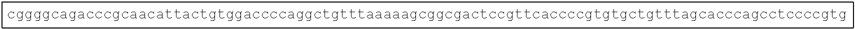

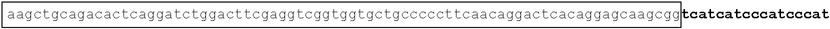

cttccacaggggcgaagagtcggtgtgtgacagtgtcagcgtgtgggttggggataagaccaccgccacagacatcaagggcaaggag
gtgatggtgttgggagaggtgaacattaacaacagtgtattcaaacagtacttttttgagaccaagtgccgggacccaaatcccgttgaca
gcgggtgccggggcattgactcaaagcactggaactcatattgtaccacgactcacacctttgtcaaggcgctgaccatggatggcaagc
aggctgcctggcggtttatccggatagatacggcctgtgtgtgtgtgctcagcaggaaggctgtgagaagagccggcggtggcggctccg
gcggtggcggctccggcggtggcggctccgacaaaactcacacatgcccaccgtgcccagcacctgaagccgctgggggaccgtcagtctt
cctcttccccccaaaacccaaggacaccctcatgatctcccggacccctgaggtcacatgcgtggtggtggacgtgagccacgaagaccctg
aggtcaagttcaactggtacgtggacggcgtggaggtgcataatgccaagacaaagccgcgggaggagcagtacaacagcacgtaccgtg
tggtcagcgtcctcaccgtcctgcaccaggactggctgaatggcaaggagtacaagtgcaaggtctccaacaaagccctcccagcctccatc
gagaaaaccatctccaaagccaaagggcagccccgagaaccacaggtgtacaccctgcccccatcccgggaggagatgaccaagaacc
aggtcagcctgacctgcctggtcaaaggcttctatcccagcgacatcgccgtggagtgggagagcaatgggcagccggagaacaactacaa
gaccacgcctcccgtgctggactccgacggctccttcttcctctatagcaagctcaccgtggacaagagcaggtggcagcaggggaacgtctt
ctcatgctccgtgatgcatgaggctctgcacaaccactacacgcagaagagcctctccctgtccccgggtaaa
SEQ ID NO: 40 (amino acid sequence of 2-L3Fc10-M3-5; signal peptide is italicized, propeptide
is squared, β-NGF (mutant 120aa) is bolded, linker is underlined, modified IgG1 Fc is italicized
and bolded)

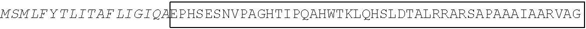

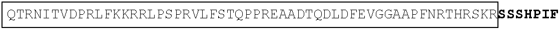

HRGEESVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVRRAGGGG
SGGGGSGGGGSDKTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPASI
EKTISKAKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGK
SEQ ID NO: 41 (nucleic acid sequence of 2-118-L3Fc10-M5-5; signal peptide is italicized,
propeptide is squared, β-NGF (mutant 118aa) is bolded, linker is underlined, modified IgG1 Fc
is italicized and bolded)

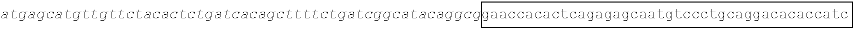

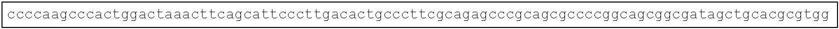

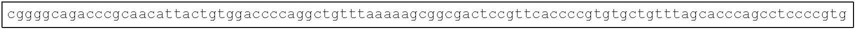

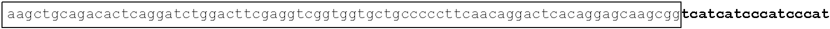

cttccacaggggcgaagagtcggtgtgtgacagtgtcagcgtgtgggttggggataagaccaccgccacagacatcaagggcaaggag
gtgatggtgttgggagaggtgaacattaacaacagtgtattcaaacagtacttttttgagaccaagtgccgggacccaaatcccgttgaca
gcgggtgccggggcattgactcaaagcactggaactcatattgtaccacgactcacacctttgtcaaggcgctgaccatggatggcaagc
aggctgcctggcggtttatccggatagatacggcctgtgtgtgtgtgctcagcaggaaggctgtgagaggcggtggcggctccggcggtgg
cggctccggcggtggcggctccGACAAAACTCACACATGCCCACCGTGCCCAGCACCTGAAGCTGAGG
GGGCACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAA
CTGGTACGTGGACGGCGTGGAGGTGCATAATGCCAAGACAAAGCCGCGGGAGGAGCAGTA
CAACAGCACGTACCGTGTGGTCAGCGTCCTCACCGTCCTGCACCAGGACTGGCTGAATGGC
AAGGAGTACAAGTGCAAGGTCTCCAACAAAGCCCTCCCAAGCTCCATCGAGAAAACCATCT
CCAAAGCCAAAGGGCAGCCCCGAGAACCACAGGTGTACACCCTGCCCCCATCCCGGGAGG
AGATGACCAAGAACCAGGTCAGCCTGACCTGCCTGGTCAAAGGCTTCTATCCCAGCGACAT
CGCCGTGGAGTGGGAGAGCAATGGGCAGCCGGAGAACAACTACAAGACCACGCCTCCCGT
GCTGGACTCCGACGGCTCCTTCTTCCTCTATAGCAAGCTCACCGTGGACAAGAGCAGGTGG
CAGCAGGGGAACGTCTTCTCATGCTCCGTGATGCATGAGGCTCTGCACAACCACTACACGC
AGAAGAGCCTCTCCCTGTCCCCGGGTAAA
SEQ ID NO: 42 (amino acid sequence of 2-118-L3Fc10-M5-5; signal peptide is italicized,
propeptide is squared, β-NGF (mutant 118aa) is bolded, linker is underlined, modified IgG1 Fc
is italicized and bolded)

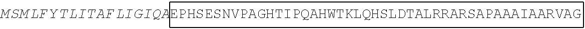

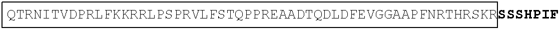

HRGEESVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVRGGGGSG
GGGSGGGGSDKTHTCPPCPAPEAEGAPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPSSIEKT
ISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
SEQ ID NO: 43 (nucleic acid sequence of 2-118-L3Fc10M7-5; signal peptide is italicized,
propeptide is squared, β-NGF (mutant 118aa) is bolded, linker is underlined, modified IgG1 Fc
is italicized and bolded)

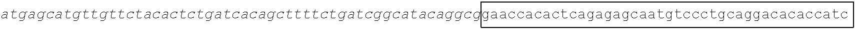

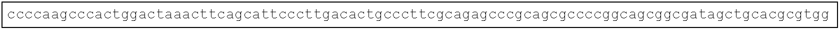

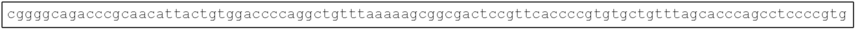

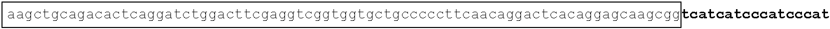

cttccacaggggcgaagagtcggtgtgtgacagtgtcagcgtgtgggttggggataagaccaccgccacagacatcaagggcaaggag
gtgatggtgttgggagaggtgaacattaacaacagtgtattcaaacagtacttttttgagaccaagtgccgggacccaaatcccgttgaca
gcgggtgccggggcattgactcaaagcactggaactcatattgtaccacgactcacacctttgtcaaggcgctgaccatggatggcaagc
aggctgcctggcggtttatccggatagatacggcctgtgtgtgtgtgctcagcaggaaggctgtgagaggcggtggcggctccggcggtgg
cggctccggcggtggcggctccGACAAAACTCACACATGCCCACCGTGCCCAGCACCTCCTGTCGCCG
GACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGAGCAGTACAAC
AGCACGTACCGTGTGGTCAGCGTCCTCACCGTCCTGCACCAGGACTGGCTGAATGGCAAGG
AGTACAAGTGCAAGGTCTCCAACAAAGGCCTCCCATCCTCCATCGAGAAAACCATCTCCAA
AGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCC
GTGGAGTGGGAGAGCAATGGGCAGCCGGAGAACAACTACAAGACCACGCCTCCCGTGCTG
GACTCCGACGGCTCCTTCTTCCTCTATAGCAAGCTCACCGTGGACAAGAGCAGGTGGCAGC
AGGGGAACGTCTTCTCATGCTCCGTGATGCATGAGGCTCTGCACAACCACTACACGCAGAA
GAGCCTCTCCCTGTCCCCGGGTAAA
SEQ ID NO: 44 (amino acid sequence of 2-118-L3Fc10M7-5; signal peptide is italicized,
propeptide is squared, β-NGF (mutant 118aa) is bolded, linker is underlined, modified IgG1 Fc
is italicized and bolded)

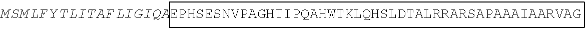

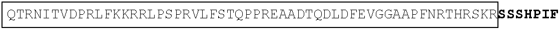

HRGEESVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVRGGGGSG
GGGSGGGGSDKTHTCPPCPAPPVAGPSVFLFPPKPKDTLMISRTPEVTCVVVDVSHEDPEVK
FNWYVDGVEVHNAKTKPREEQYNSTYRVVSVLTVLHQDWLNGKEYKCKVSNKGLPSSIEKTI
SKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
SEQ ID NO: 45 (nucleic acid sequence of 2-118-L3G4-BM; signal peptide is italicized,
propeptide is squared, β-NGF (mutant 118aa) is bolded, linker is underlined, modified IgG4 Fc
is italicized and bolded)

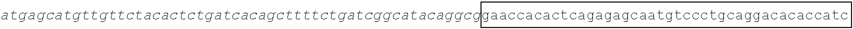

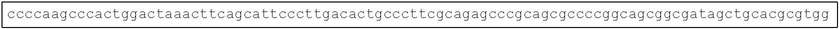

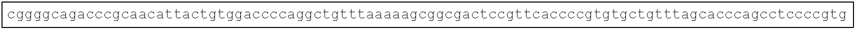

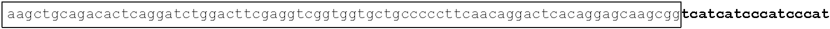

cttccacaggggcgaagagtcggtgtgtgacagtgtcagcgtgtgggttggggataagaccaccgccacagacatcaagggcaaggag
gtgatggtgttgggagaggtgaacattaacaacagtgtattcaaacagtacttttttgagaccaagtgccgggacccaaatcccgttgaca
gcgggtgccggggcattgactcaaagcactggaactcatattgtaccacgactcacacctttgtcaaggcgctgaccatggatggcaagc
aggctgcctggcggtttatccggatagatacggcctgtgtgtgtgtgctcagcaggaaggctgtgagaGGAGGGGGAGGCGG
AGGTTCAGGGGGTGGTGGTTCCGGcGGCGGGGGATCCGCCgagtccaaatatggtcccccatgcccaccct
gcccagcacctgaggctgcggggggaccatcagtcttcctgttccccccaaaacccaaggacactctcatgatctcccggacccctgaggtca
cgtgcgtggtggtggacgtgagccaggaagaccccgaggtccagttcaactggtacgtggatggcgtggaggtgcataatgccaagacaaa
gccgcgggaggagcagttcaacagcacgtaccgtgtggtcagegtcctcaccgtcctgcaccaggactggctgaacggcaaggagtacaa
gtgcaaggtctccaacaaaggcctcccgtcctccatcgagaaaaccatctccaaagccaaagggcagccccgagagccacaggtgtacac
cctgcccccatcccaggaggagatgaccaagaaccaggtcagcctgacctgcctggtcaaaggcttctaccccagcgacatcgccgtggag
tgggaaagcaatgggcagccggagaacaactacaagaccacgcctcccgtgctggactccgacggctccttcttcctctacagcaggctaac
cgtggacaagagcaggtggcaggaggggaatgtcttctcatgctccgtgatgcatgaggctctgcacaaccactacacacagaagagcctct
ccctgtctctgggtaaa
SEQ ID NO: 46 (amino acid sequence of 2-118-L3G4-BM; signal peptide is italicized,
propeptide is squared, β-NGF (mutant 118aa) is bolded, linker is underlined, modified IgG4 Fc
is italicized and bolded)

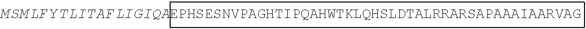

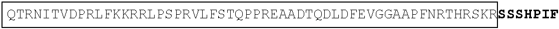

HRGEESVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVRGGGGGG
SGGGGSGGGGSAESKYGPPCPPCPAPEAAGGPSVFLFPPKPKDTLMISRTPEVTCVVVDVSQE
DPEVQFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLSLSLGK
SEQ ID NO: 47 (mutant human preproNGF, 239aa; signal peptide is italicized, propeptide is
squared, β-NGF (mutant 118aa) is bolded)

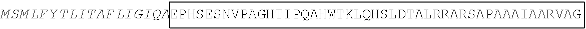

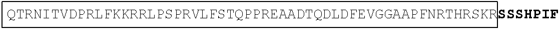

HRGEESVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVR
SEQ ID NO: 48 (mutant human preproNGF, 241aa; signal peptide is italicized, propeptide is
squared, β-NGF (mutant 120aa) is bolded)

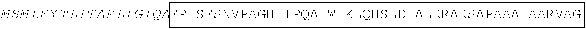

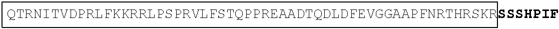

HRGEESVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVRRA
SEQ ID NO: 49 (wildtype human preproNGF, 239aa; signal peptide is italicized, propeptide is
squared, β-NGF (wt 118aa) is bolded)

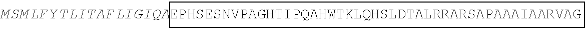

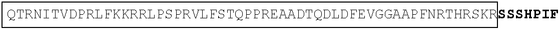

HRGEFSVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVR
SEQ ID NO: 50 (wildtype human preproNGF, 241aa; signal peptide is italicized, propeptide is
squared, β-NGF (wt 120aa) is bolded)

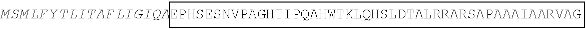

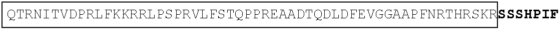

HRGEFSVCDSVSVWVGDKTTATDIKGKEVMVLGEVNINNSVFKQYFFETKCRDPNPVDS
GCRGIDSKHWNSYCTTTHTFVKALTMDGKQAAWRFIRIDTACVCVLSRKAVRRA
SEQ ID NO: 51 (mutant human proNGF, 221aa; propeptide is squared, β-NGF (mutant 118aa)
is bolded)

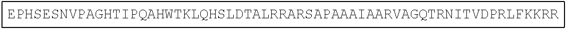

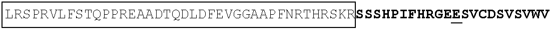

GDKTTATDIKGKEVMVLGEVNINNSVFKQYFFETKCRDPNPVDSGCRGIDSKHWNSYCT
TTHTFVKALTMDGKQAAWRFIRIDTACVCVLSRKAVR
SEQ ID NO: 52 (mutant human proNGF, 223aa; propeptide is squared, β-NGF (mutant 120aa)
is bolded)

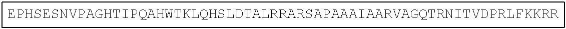

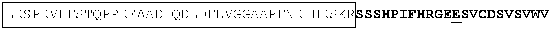

GDKTTATDIKGKEVMVLGEVNINNSVFKQYFFETKCRDPNPVDSGCRGIDSKHWNSYCT
TTHTFVKALTMDGKQAAWRFIRIDTACVCVLSRKAVRRA
SEQ ID NO: 53 (wildtype human proNGF, 221aa; propeptide is squared, β-NGF (wt 118aa) is
bolded)

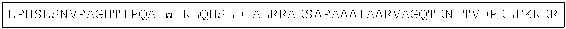

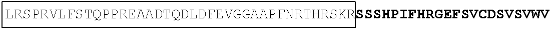

GDKTTATDIKGKEVMVLGEVNINNSVFKQYFFETKCRDPNPVDSGCRGIDSKHWNSYCT
TTHTFVKALTMDGKQAAWRFIRIDTACVCVLSRKAVR
SEQ ID NO: 54 (wildtype human proNGF, 223aa; propeptide is squared, β-NGF (wt 120aa) is
bolded)

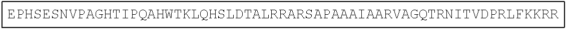

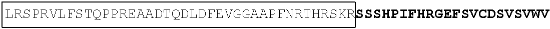

GDKTTATDIKGKEVMVLGEVNINNSVFKQYFFETKCRDPNPVDSGCRGIDSKHWNSYCT
TTHTFVKALTMDGKQAAWRFIRIDTACVCVLSRKAVRRA
SEQ ID NO: 55 (amino acid sequence of mature FD-G4Fc; β-NGF (wt 118aa) is bolded, linker is
underlined, modified IgG4 Fc is italicized and bolded)
SSSHPIFHRGEFSVCDSVSVWVGDKTTATDIKGKEVMVLGEVNINNSVFKQYFFETKCRD
PNPVDSGCRGIDSKHWNSYCTTTHTFVKALTMDGKQAAWRFIRIDTACVCVLSRKAVRG
SGGGSGGGGSGGGGSGGGGSSKYGPPCPPCPAPEFEGGPSVFLFPPKPKDTLMISRTPEVTC
VVVDVSQEDPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDIAVEWESN
GQPENNYKTTPPVLDSDGSFFLYSRLTVDKSRWQEGNVFSCSVMHEALHNHYTQKSLSLSPGK
SEQ ID NO: 56 (amino acid sequence of mature WM-G24Fc; β-NGF (wt 118aa) is bolded,
linker is underlined, modified IgG2/4 Fc is italicized and bolded)
SSSHPIFHRGEFSVCDSVSVWVGDKTTATDIKGKEVMVLGEVNINNSVFKQYFFETKCRD
PNPVDSGCRGIDSKHWNSYCTTTHTFVKALTMDGKQAAWRFIRIDTACVCVLSRKAVRK
TGGGSGGGSVERKCCVECPPCPAPPVAGPSVFLFPPKPKDTLMISRTPEVTCVVVDVSQEDPE
VQFNWYVDGVEVHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGK
SEQ ID NO: 57 (amino acid sequence of mature NGF-1-15M7; β-NGF (wt 120aa) is bolded,
modified IgG1 Fc is italicized and bolded)
SSSHPIFHRGEFSVCDSVSVWVGDKTTATDIKGKEVMVLGEVNINNSVFKQYFFETKCRD
PNPVDSGCRGIDSKHWNSYCTTTHTFVKALTMDGKQAAWRFIRIDTACVCVLSRKAVRR
AEPKSCDKTHTCPPCPAPPVAGPSVFLFPPKPKDTLMISRTPEVTCVVVDVSHEDPEVKFNW
YVDGVEVHNAKTKPREEQYNSTYRVVSVLTVLHQDWLNGKEYKCKVSNKGLPSSIEKTISKAK
GQPREPQVYTLPPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGK
SEQ ID NO: 58 (amino acid sequence of mature NGF-L3Fc10M7-5; β-NGF (wt 120aa) is
bolded, linker is underlined, modified IgG1 Fc is italicized and bolded)
SSSHPIFHRGEFSVCDSVSVWVGDKTTATDIKGKEVMVLGEVNINNSVFKQYFFETKCRD
PNPVDSGCRGIDSKHWNSYCTTTHTFVKALTMDGKQAAWRFIRIDTACVCVLSRKAVRR
AGGGGSGGGGSGGGGSDKTHTCPPCPAPPVAGPSVFLFPPKPKDTLMISRTPEVTCVVVDVS
HEDPEVKFNWYVDGVEVHNAKTKPREEQYNSTYRVVSVLTVLHQDWLNGKEYKCKVSNKG
LPSSIEKTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPGK
SEQ ID NO: 59 (amino acid sequence of mature 2-1-15M7; β-NGF (mutant 120aa) is bolded,
modified IgG1 Fc is italicized and bolded)
SSSHPIFHRGEESVCDSVSVWVGDKTTATDIKGKEVMVLGEVNINNSVFKQYFFETKCR
DPNPVDSGCRGIDSKHWNSYCTTTHTFVKALTMDGKQAAWRFIRIDTACVCVLSRKAVR
RAEPKSCDKTHTCPPCPAPPVAGPSVFLFPPKPKDTLMISRTPEVTCVVVDVSHEDPEVKEN
WYVDGVEVHNAKTKPREEQYNSTYRVVSVLTVLHQDWLNGKEYKCKVSNKGLPSSIEKTISK
AKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 60 (amino acid sequence of mature NGF-4-12PAA; β-NGF (wt 120aa) is bolded,
modified IgG4 Fc is italicized and bolded)
SSSHPIFHRGEFSVCDSVSVWVGDKTTATDIKGKEVMVLGEVNINNSVFKQYFFETKCRD
PNPVDSGCRGIDSKHWNSYCTTTHTFVKALTMDGKQAAWRFIRIDTACVCVLSRKAVRR
AESKYGPPCPPCPAPEAAGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
SEQ ID NO: 61 (amino acid sequence of mature 2-118-L3Fc10-M1-5; β-NGF (mutant 118aa) is
bolded, linker is underlined, modified IgG1 Fc is italicized and bolded)
SSSHPIFHRGEESVCDSVSVWVGDKTTATDIKGKEVMVLGEVNINNSVFKQYFFETKCR
DPNPVDSGCRGIDSKHWNSYCTTTHTFVKALTMDGKQAAWRFIRIDTACVCVLSRKAVR
GGGGSGGGGSGGGGSDKTHTCPPCPAPELLGGPSVFLFPPKPKDTLMISRTPEVTCVVVDVS
HEDPEVKFNWYVDGVEVHNAKTKPREEQYASTYRVVSVLTVLHQDWLNGKEYKCKVSNKAL
PAPIEKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK
SEQ ID NO: 62 (amino acid sequence of mature 2-118-L3Fc10-M3-5; β-NGF (mutant 118aa) is
bolded, linker is underlined, modified IgG1 Fc is italicized and bolded)
SSSHPIFHRGEESVCDSVSVWVGDKTTATDIKGKEVMVLGEVNINNSVFKQYFFETKCR
DPNPVDSGCRGIDSKHWNSYCTTTHTFVKALTMDGKQAAWRFIRIDTACVCVLSRKAVR
GGGGSGGGGSGGGGSDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKAL
PASIEKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK
SEQ ID NO: 63 (amino acid sequence of mature NGF-118-L3Fc10-M3-5; β-NGF (wt 118aa) is
bolded, linker is underlined, modified IgG1 Fc is italicized and bolded)
SSSHPIFHRGEFSVCDSVSVWVGDKTTATDIKGKEVMVLGEVNINNSVFKQYFFETKCRD
PNPVDSGCRGIDSKHWNSYCTTTHTFVKALTMDGKQAAWRFIRIDTACVCVLSRKAVRG
GGGSGGGGSGGGGSDKTHTCPPCPAPEAAGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALP
ASIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
SEQ ID NO: 64 (amino acid sequence of mature 2-L3Fc10-M3-5; β-NGF (mutant 120aa) is
bolded, linker is underlined, modified IgG1 Fc is italicized and bolded)
SSSHPIFHRGEESVCDSVSVWVGDKTTATDIKGKEVMVLGEVNINNSVFKQYFFETKCR
DPNPVDSGCRGIDSKHWNSYCTTTHTFVKALTMDGKQAAWRFIRIDTACVCVLSRKAVR
RAGGGGSGGGGSGGGGSDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLNGKEYKCKVSNK
ALPASIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK
SEQ ID NO: 65 (amino acid sequence of mature 2-118-L3Fc10-M5-5; β-NGF (mutant 118aa) is
bolded, linker is underlined, modified IgG1 Fc is italicized and bolded)
SSSHPIFHRGEESVCDSVSVWVGDKTTATDIKGKEVMVLGEVNINNSVFKQYFFETKCR
DPNPVDSGCRGIDSKHWNSYCTTTHTFVKALTMDGKQAAWRFIRIDTACVCVLSRKAVR
GGGGSGGGGSGGGGSDKTHTCPPCPAPEAEGAPSVFLFPPKPKDTLMISRTPEVTCVVVDVS
HEDPEVKFNWYVDGVEVHNAKTKPREEQYNSTYRVVSVLTVLHQDWLNGKEYKCKVSNKAL
PSSIEKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK
SEQ ID NO: 66 (amino acid sequence of mature 2-118-L3Fc10M7-5; β-NGF (mutant 118aa) is
bolded, linker is underlined, modified IgG1 Fc is italicized and bolded)
SSSHPIFHRGEESVCDSVSVWVGDKTTATDIKGKEVMVLGEVNINNSVFKQYFFETKCR
DPNPVDSGCRGIDSKHWNSYCTTTHTFVKALTMDGKQAAWRFIRIDTACVCVLSRKAVR
GGGGSGGGGSGGGGSDKTHTCPPCPAPPVAGPSVFLFPPKPKDTLMISRTPEVTCVVVDVSH
EDPEVKFNWYVDGVEVHNAKTKPREEQYNSTYRVVSVLTVLHQDWLNGKEYKCKVSNKGLP
SSIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
SEQ ID NO: 67 (amino acid sequence of mature 2-118-L3G4-BM; B-NGF (mutant 118aa) is
bolded, linker is underlined, modified IgG4 Fc is italicized and bolded)
SSSHPIFHRGEESVCDSVSVWVGDKTTATDIKGKEVMVLGEVNINNSVFKQYFFETKCR
DPNPVDSGCRGIDSKHWNSYCTTTHTFVKALTMDGKQAAWRFIRIDTACVCVLSRKAVR
GGGGGGSGGGGSGGGGSAESKYGPPCPPCPAPEAAGGPSVFLFPPKPKDTLMISRTPEVTCV
VVDVSQEDPEVQFNWYVDGVEVHNAKTKPREEQFNSTYRVVSVLTVLHQDWLNGKEYKCK
VSNKGLPSSIEKTISKAKGQPREPQVYTLPPSQEEMTKNQVSLTCLVKGFYPSDIAVEWESNG
QPENNYKTTPPVLDSDGSFFLYSRLTVDKSRWQEGNVFSCSVMHEALHNHYTQKSLSLSLGK
SEQ ID NO: 68 (linker)
GGGGSGGGGSGGGGS
SEQ ID NO: 69 (linker)
GGGGGGSGGGGSGGGGSA
SEQ ID NO: 70 (linker; n is an integer of at least 1)
(GGGGS)ₙ
SEQ ID NO: 71 (linker)
GSGGGSGGGGSGGGGSGGGGS
SEQ ID NO: 72 (linker)
KTGGGSGGGS
SEQ ID NO: 73 (linker; n is an integer of at least 1)
(G)ₙ
SEQ ID NO: 74 (linker; n is an integer of at least 1)
(GS)ₙ
SEQ ID NO: 75 (linker; n is an integer of at least 1)
(GGS)ₙ
SEQ ID NO: 76 (linker; n is an integer of at least 1)
(GGGS)ₙ
SEQ ID NO: 77 (linker; n is an integer of at least 1)
(GGS),(GGGS)ₙ
SEQ ID NO: 78 (linker; n is an integer of at least 1)
(GSGGS)ₙ
SEQ ID NO: 79 (linker; n is an integer of at least 1)
(GGSGS)ₙ
SEQ ID NO: 80 (linker)
GSGGGSGGGGSGGGGS
SEQ ID NO: 81 (linker)
GGGSGGGGSGGGGS
SEQ ID NO: 82 (linker)
GGGSGGSGGS
SEQ ID NO: 83 (linker)
GGSGGSGGSGGSGGG
SEQ ID NO: 84 (linker)
GGSGGSGGGGSGGGGS
SEQ ID NO: 85 (linker)
GGSGGSGGSGGSGGSGGS
SEQ ID NO: 86 (linker)
GG
SEQ ID NO: 87 (linker)
GGSG
SEQ ID NO: 88 (linker)
GGSGG
SEQ ID NO: 89 (linker)
GSGSG
SEQ ID NO: 90 (linker)
GSGGG
SEQ ID NO: 91 (linker)
GGGSG
SEQ ID NO: 92 (linker)
GSSSG
SEQ ID NO: 93 (linker)
GGSGGS
SEQ ID NO: 94 (linker)
SGGGGS
SEQ ID NO: 95 (linker)
GGGGS
SEQ ID NO: 96 (linker; n is an integer of at least 1)
(GA)ₙ
SEQ ID NO: 97 (linker)
GRAGGGGAGGGG
SEQ ID NO: 98 (linker)
GRAGGG
SEQ ID NO: 99 (linker)
ASTKGP

1. A long-acting nerve growth factor (NGF) polypeptide comprising from N-terminus to C-terminus an NGF moiety and an Fc moiety, wherein the NGF moiety comprises the amino acid sequence of any one of SEQ ID NOs: 1-3, and wherein the Fc moiety is derived from an IgG1 Fc or an IgG4 Fc.

2. The long-acting NGF polypeptide of claim 1, wherein the NGF moiety is fused to the Fc moiety via a peptide linker, wherein
(i) the peptide linker comprises the amino acid sequence of any one of SEQ ID NOs: 68-72, and/or
(ii) the peptide linker comprises the amino acid sequence of (GGGGS)ₙ (SEQ ID NO: 70), and wherein n is any of 1, 2, 3, 4, 5, or 6.

3-4. (canceled)

5. The long-acting NGF polypeptide of claim 1, wherein the Fc moiety is derived from an IgG1 Fc, wherein
(i) the Fc comprises the amino acid sequence of SEQ ID NO: 7 or 8, and/or
(ii) the Fc moiety comprises a mutation at a position selected from one or more of E233, L234, L235, G236, G237, N297, A327, A330, and P331 relative to SEQ ID NO: 8, and/or
(iii) the Fc moiety comprises a mutation selected from one or more of E233P, L234V, L234A, L235A, L235E, G236del, G237A, N297A, A327G, A330S, and P331S relative to SEQ ID NO: 8, and/or
(iv) the Fc moiety further lacks the first 5 amino acids of SEQ ID NO: 7 or 8.

6-8. (canceled)

9. The long-acting NGF polypeptide of claim 5, wherein
(i) the Fc moiety comprises L234A, L235A, and P331S mutations relative to SEQ ID NO: 8, and/or
(ii) the Fc moiety comprises the amino acid sequence of SEQ ID NO: 11 or 12.

10. (canceled)

11. The long-acting NGF polypeptide of claim 9, wherein the long-acting NGF polypeptide comprises the amino acid sequence of any one of SEQ ID NOs: 62-64.

12. The long-acting NGF polypeptide of claim 5, wherein
(i) the Fc moiety comprises E233P, L234V, L235A, G236del, A327G, A330S, and P331S mutations relative to SEQ ID NO: 8, and/or
(ii) the Fc moiety comprises the amino acid sequence of SEQ ID NO: 15 or 16.

13. (canceled)

14. The long-acting NGF polypeptide of claim 12, wherein the long-acting NGF polypeptide comprises the amino acid sequence of SEQ ID NO: 66.

15. The long-acting NGF polypeptide of claim 5, wherein
(i) the Fc moiety comprises L234A, L235E, G237A, A330S, and P331S mutations relative to SEQ ID NO: 8, and/or
(ii) the Fc moiety comprises the amino acid sequence of SEQ ID NO: 13 or 14.

16. (canceled)

17. The long-acting NGF polypeptide of claim 15, wherein the long-acting NGF polypeptide comprises the amino acid sequence of SEQ ID NO: 65.

18. The long-acting NGF polypeptide of claim 5, wherein
(i) the Fc moiety comprises an N297A mutation relative to SEQ ID NO: 8, and/or
(ii) the Fc moiety comprises the amino acid sequence of SEQ ID NO: 9 or 10.

19. (canceled)

20. The long-acting NGF polypeptide of claim 18, wherein the long-acting NGF polypeptide comprises the amino acid sequence of SEQ ID NO: 61.

21. The long-acting NGF polypeptide of claim 1, wherein the Fc moiety is derived from an IgG4 Fc, wherein
(i) the Fc comprises the amino acid sequence of SEQ ID NO: 17, and/or
(ii) the Fc moiety comprises a mutation at a position selected from one or more of S228, F234, and L235 relative to SEQ ID NO: 17, and/or
(iii) the Fc moiety comprises a mutation selected from one or more of S228P, F234A, and L235A relative to SEQ ID NO: 17, and/or
(iv) the Fc moiety comprises the amino acid sequence of SEQ ID NO: 18.

22-24. (canceled)

25. The long-acting NGF polypeptide of claim 21, wherein the long-acting NGF polypeptide comprises the amino acid sequence of SEQ ID NO: 67.

26. The long-acting NGF polypeptide of claim 1, wherein
(i) the long-acting NGF polypeptide has a half-life of at least about 10 hours when administered to a human individual intravenously, intramuscularly, intraocularly, or subcutaneously, and/or
(ii) the long-acting NGF polypeptide causes less pain compared to an NGF polypeptide comprising an NGF moiety with the amino acid sequence of SEQ ID NO: 3 or 4.

27. (canceled)

28. An isolated nucleic acid encoding the long-acting NGF polypeptide of claim 1.

29. A vector comprising the nucleic acid of claim 28.

30. A host cell comprising the vector of claim 29.

31. A pharmaceutical composition comprising the long-acting NGF polypeptide of claim 1, and optionally a pharmaceutically acceptable carrier and/or excipient.

32. A method of treating an NGF-related disease in an individual, comprising administering to the individual an effective amount of the pharmaceutical composition of claim 31, wherein the NGF-related disease is preferably a neurological disease or a non-neurological disease;
more preferably, the neurological disease is selected from the group consisting of neonatal hypoxic-ischemic encephalopathy, cerebral palsy, critical illness myopathy, nerve deafness, recurrent laryngeal nerve injury, traumatic brain injury, tooth nerve injury, cerebral stroke, Down syndrome, amyotrophic lateral sclerosis, multiple sclerosis, spinal muscular atrophy, diffuse brain injury, thymus dysplasia, optic contusion, follicular dysplasia, spinal cord injury, glaucoma, neurotrophic keratitis, optic injury, neuromyelitis optica, retinal associated diseases, urinary incontinence, Alzheimer's disease, Parkinson's disease, Huntington's disease, dementia, Hypertensive Intracerebral Hemorrhage Neurological Dysfunction, Cerebral Small Vessel Disease, Acute Ischemic Stroke, corneal endothelial dystrophy, diabetic neuropathy, diabetic foot ulcer, neurogenic skin ulcer, pressure sore, neurotrophic corneal ulcer, diabetic corneal ulcer, and macular hole, and/or
the non-neurological disease is selected from the group consisting of atrophy of spleen, contusion of spleen, diminished ovarian reserve, premature ovarian failure, Ovarian Hyperstimulation Syndrome, ovarian remnant syndrome, ovarian follicular dysplasia, spermatogenesis disorder such as oligozoospermia, asthenospermia, and oligoasthenospermia, ischemic ulcer, stress ulcer, rheumatoid ulcer, liver fibrosis, corneal ulcer, burns, oral ulcer, and venous leg ulcers;
even more preferably, the neurological disease is diabetic neuropathy, Alzheimer's disease, or neurotrophic keratitis, and/or
the non-neurological disease is premature ovarian failure or spermatogenesis disorder.

33-38. (canceled)

39. The method of claim 32, wherein
(i) the pharmaceutical composition is administered at a dose of about 0.01 μg to about 1000 μg per individual, and/or
(ii) the pharmaceutical composition is administered at a dosing frequency of about once every week or about once per month, and/or
(iii) the pharmaceutical composition is administered orally, subcutaneously, intravenously, intracerebrally, intranasally, transdermally, intraperitoneally, intramuscularly, intrapulmonarily, vaginally, rectally, intraocularly, or topically.

40-41. (canceled)
